(19) United States
(12) Patent Application Publication
(10) Pub. No.: US 2010/0331315 A1
(43) Pub. Date: Dec. 30, 2010

(54) RHODANINES AND RELATED HETEROCYCLES AS KINASE INHIBITORS

(76) Inventors: Mustapha HADDACH, San Diego, CA (US); David M. RYCKMAN, San Diego, CA (US)

Correspondence Address: COOLEY LLP;ATTN: Patent Group, Suite 1100, 777 - 6th Street, NW, WASHINGTON, DC, 20001, US

(21) Appl. No.: 12/819,091

(22) Filed: Jun. 18, 2010

Related U.S. Application Data
(60) Provisional application No. 61/218,314, filed on Jun. 18, 2009.

(51) Int. Cl. A61K 31/551; C07D 405/06; C07D 405/14; C07D 417/14; C07D 403/14; C07D 409/14; C07D 417/10; A61K 31/427; A61K 31/496; A61K 31/454; A61K 31/4709; A61K 31/497; A61P 35/00; A61P 9/00; A61P 29/00; A61P 37/02; A61P 31/00

(52) U.S. Cl. 514/218; 548/183; 540/575; 544/369; 546/209; 546/162; 544/405; 544/336; 514/369; 514/254.02; 514/326; 514/313; 514/255.05

(57) ABSTRACT

The invention provides compounds that inhibit PIM kinases and/or CK2, and compositions containing such compounds. These compounds and compositions are useful for treating proliferative disorders such as cancer, as well as other kinase-associated conditions including inflammation, pain, vascular disorders, pathogenic infections and certain immunological disorders.

### CROSS-REFERENCE TO RELATED APPLICATION

This application claims the benefit of priority to U.S. Provisional Application Ser. No. 61/218,314, filed 18 Jun. 2009. The content of this document is incorporated herein by reference in its entirety.

### FIELD OF THE INVENTION

The invention relates in part to molecules having certain biological activities that include, but are not limited to, inhibiting cell proliferation, and modulating certain protein kinase activities. Molecules of the invention can modulate casein kinase activity (e.g., CK2 activity) and/or Pim kinase activity (e.g., PIM-1 activity), and are useful to treat cancers and inflammatory conditions as well as certain infectious disorders. The invention also relates in part to methods for using such compounds, and pharmaceutical compositions containing these compounds.

### BACKGROUND

Protein kinase CK2 (formerly called Casein kinase II, referred to herein as “CK2”) is a ubiquitous and highly conserved protein serine/threonine kinase. The holoenzyme is typically found in tetrameric complexes consisting of two catalytic (alpha and/or alpha') subunits and two regulatory (beta) subunits. CK2 has a number of physiological targets and participates in a complex series of cellular functions including the maintenance of cell viability. The level of CK2 in normal cells is tightly regulated, and it has long been considered to play a role in cell growth and proliferation Inhibitors of CK2 that are useful for treating certain types of cancers are described in PCT/US2007/077464, PCT/US2008/074820, PCT/US2009/35609.

Both the prevalence and the importance of CK2 suggest it is an ancient enzyme on the evolutionary scale, as does an evolutionary analysis of its sequence; its longevity may explain why it has become important in so many biochemical processes, and why CK2 from hosts have even been co-opted by infectious pathogens (e.g., viruses, protozoa) as an integral part of their survival and life cycle biochemical systems. These same characteristics explain why inhibitors of CK2 are believed to be useful in a variety of medical treatments as discussed herein. Because it is central to many biological processes, as summarized by Guerra & Issinger, Curr. Med. Chem., 2008, 15:1870-1886, inhibitors of CK2, including the compounds described herein, should be useful in the treatment of a variety of diseases and disorders.

Cancerous cells show an elevation of CK2, and recent evidence suggests that CK2 exerts potent suppression of apoptosis in cells by protecting regulatory proteins from caspase-mediated degradation. The anti-apoptotic function of CK2 may contribute to its ability to participate in transformation and tumorigenesis. In particular, CK2 has been shown to be associated with acute and chronic myelogenous leukemia, lymphoma and multiple myeloma. In addition, enhanced CK2 activity has been observed in solid tumors of the colon, rectum and breast, squamous cell carcinomas of the lung and of the head and neck (SCCHN), adenocarcinomas of the lung, colon, rectum, kidney, breast, and prostate Inhibition of CK2 by a small molecule is reported to induce apoptosis of pancreatic cancer cells, and hepatocellular carcinoma cells (HegG2, Hep3, HeLa cancer cell lines); and CK2 inhibitors dramatically sensitized RMS (Rhabdomyosarcoma) tumors toward apoptosis induced by TRAIL. Thus an inhibitor of CK2 alone, or in combination with TRAIL or a ligand for the TRAIL receptor, would be useful to treat RMS, the most common soft-tissue sarcoma in children. In addition, elevated CK2 has been found to be highly correlated with aggressiveness of neoplasias, and treatment with a CK2 inhibitor of the invention should thus reduce tendency of benign lesions to advance into malignant ones, or for malignant ones to metastasize.

Unlike other kinases and signaling pathways, where mutations are often associated with structural changes that cause loss of regulatory control, increased CK2 activity level appears to be generally caused by upregulation or overexpression of the active protein rather than by changes that affect activation levels. Guerra and Issinger postulate this may be due to regulation by aggregation, since activity levels do not correlate well with mRNA levels. Excessive activity of CK2 has been shown in many cancers, including SCCHN tumors, lung tumors, breast tumors, and others. Id.

Elevated CK2 activity in colorectal carcinomas was shown to correlate with increased malignancy. Aberrant expression and activity of CK2 have been reported to promote increase nuclear levels of NF-kappaB in breast cancer cells. CK2 activity is markedly increased in patients with AML and CML during blast crisis, indicating that an inhibitor of CK2 should be particularly effective in these conditions. Multiple myeloma cell survival has been shown to rely on high activity of CK2, and inhibitors of CK2 were cytotoxic to MM cells. Similarly, a CK2 inhibitor inhibited growth of murine p190 lymphoma cells. Its interaction with Bcr/Abl has been reported to play an important role in proliferation of Bcr/Abl expressing cells, indicating inhibitors of CK2 may be useful in treatment of Bcr/Abl-positive leukemias Inhibitors of CK2 have been shown to inhibit progression of skin papillomas, prostate and breast cancer xenografts in mice, and to prolong survival of transgenic mice that express prostate-promoters. Id.

The role of CK2 in various non-cancer disease processes has been recently reviewed. See Guerra & Issinger, Curr. Med. Chem., 2008, 15:1870-1886. Increasing evidence indicates that CK2 is involved in critical diseases of the central nervous system, including, for example, Alzheimer's disease, Parkinson's disease, and rare neurodegenerative disorders such as Guam-Parkinson dementia, chromosome 18 deletion syndrome, progressive supranuclear palsy, Kuf's disease, or Pick's disease. It is suggested that selective CK2-mediated phosphorylation of tau proteins may be involved in progressive neurodegeneration of Alzheimer's. In addition, recent studies suggest that CK2 plays a role in memory impairment and brain ischemia, the latter effect apparently being mediated by CK2's regulatory effect on the PI3K survival pathways.

CK2 has also been shown to be involved in the modulation of inflammatory disorders, for example, acute or chronic inflammatory pain, glomerulonephritis, and autoimmune diseases, including, e.g., multiple sclerosis (MS), systemic lupus erythematosus, rheumatoid arthritis, and juvenile arthritis. It positively regulates the function of the serotonin 5-HT3 receptor channel, activates heme oxygenase type 2, and enhances the activity of neuronal nitric oxide synthase. A selective CK2 inhibitor was reported to strongly reduce pain response of mice when administered to spinal cord tissue prior to pain testing. It phosphorylates secretory type IIA phospholipase A2 from synovial fluid of RA patients, and modulates secretion of DEK (a nuclear DNA-binding protein), which is a proinflammatory molecule found in synovial fluid of patients with juvenile arthritis. Thus inhibition of CK2 is expected to control progression of inflammatory pathologies such as those described here, and the inhibitors disclosed herein have been shown to effectively treat pain in animal models.

Protein kinase CK2 has also been shown to play a role in disorders of the vascular system, such as, e.g., atherosclerosis, laminar shear stress, and hypoxia. CK2 has also been shown to play a role in disorders of skeletal muscle and bone tissue, such as cardiomyocyte hypertrophy, impaired insulin signaling and bone tissue mineralization. In one study, inhibitors of CK2 were effective at slowing angiogenesis induced by growth factor in cultured cells. Moreover, in a retinopathy model, a CK2 inhibitor combined with octreotide (a somatostatin analog) reduced neovascular tufts; thus the CK2 inhibitors described herein would be effective in combination with a somatostatin analog to treat retinopathy.

CK2 has also been shown to phosphorylate GSK, troponin and myosin light chain; thus it is important in skeletal muscle and bone tissue physiology, and is linked to diseases affecting muscle tissue.

Evidence suggests that CK2 is also involved in the development and life cycle regulation of protozoal parasites, such as, for example, Theileria parva, Trypanosoma cruzi, Leishmania donovani, Herpetomonas muscarum muscarum, Plasmodium falciparum, Trypanosoma brucei, Toxoplasma gondii and Schistosoma mansoni. Numerous studies have confirmed the role of CK2 in regulation of cellular motility of protozoan parasites, essential to invasion of host cells. Activation of CK2 or excessive activity of CK2 has been shown to occur in hosts infected with Leishmania donovani, Herpetomonas muscarum muscarum, Plasmodium falciparum, Trypanosoma brucei, Toxoplasma gondii and Schistosoma mansoni. Indeed, inhibition of CK2 has been shown to block infection by T. cruzi.

CK2 has also been shown to interact with and/or phosphorylate viral proteins associated with human immunodeficiency virus type 1 (HIV-1), human papilloma virus, and herpes simplex virus, in addition to other virus types (e.g. human cytomegalovirus, hepatitis C and B viruses, Borna disease virus, adenovirus, coxsackievirus, coronavirus, influenza, and varicella zoster virus). CK2 phosphorylates and activates HIV-1 reverse transcriptase and proteases in vitro and in vivo, and promotes pathogenicity of simian-human immunodeficiency virus (SHIV), a model for HIV. Inhibitors of CK2 are thus able to reduce pathogenic effects of a model of HIV infection. CK2 also phosphorylates numerous proteins in herpes simplex virus and numerous other viruses, and some evidence suggests viruses have adopted CK2 as a phosphorylating enzyme for their essential life cycle proteins Inhibition of CK2 is thus expected to deter infection and progression of viral infections, which rely upon the host's CK2 for their own life cycles.

CK2 is unusual in the diversity of biological processes that it affects, and it differs from most kinases in other ways as well: it is constitutively active, it can use ATP or GTP, and it is elevated in most tumors and rapidly proliferating tissues. It also has unusual structural features that may distinguish it from most kinases, too, enabling its inhibitors to be highly specific for CK2 while many kinase inhibitors affect multiple kinases, increasing the likelihood of off-target effects, or variability between individual subjects. For all of these reasons, CK2 is a particularly interesting target for drug development, and the invention provides highly effective inhibitors of CK2 that are useful in treating a variety of different diseases and disorders mediated by or associated with excessive, aberrant or undesired levels of CK2 activity.

The PIM protein kinases which include the closely related PIM-1, -2, and -3, have been implicated in diverse biological processes such as cell survival, proliferation, and differentiation. PIM-1 is involved in a number of signaling pathways that are highly relevant to tumorigenesis [reviewed in Bachmann & Moroy, Internat. J. Biochem. Cell Biol., 37, 726-730 (2005)]. Many of these are involved in cell cycle progression and apoptosis. It has been shown that PIM-1 acts as an anti-apoptotic factor via inactivation of the pro-apoptotic factor BAD (Bcl2 associated death promoter, an apoptosis initiator). This finding suggested a direct role of PIM-1 in preventing cell death, since the inactivation of BAD can enhance Bcl-2 activity and can thereby promote cell survival [Aho et al., FEBS Letters, 571, 43-49 (2004)]. PIM-1 has also been recognized as a positive regulator of cell cycle progression. PIM-1 binds and phosphorylates Cdc25A, which leads to an increase in its phosphatase activity and promotion of G1/S transition [reviewed in Losman et al., JBC, 278, 4800-4805 (1999)]. In addition, the cyclin kinase inhibitor p21^{Waf} which inhibits G1/S progression, was found to be inactivated by PIM-1 [Wang et al., Biochim. Biophys. Act. 1593, 45-55 (2002)]. Furthermore, by means of phosphorylation, PIM-1 inactivates C-TAK1 and activates Cdc25C which results in acceleration of G2/M transition [Bachman et al., JBC, 279, 48319-48 (2004)].

PIM-1 appears to be an essential player in hematopoietic proliferation. Kinase active PIM-1 is required for the gp130-mediated STAT3 proliferation signal [Hirano et al., Oncogene 19, 2548-2556, (2000)]. PIM-1 is overexpressed or even mutated in a number of tumors and different types of tumor cell lines and leads to genomic instability. Fedorov, et al., concluded that a Phase III compound in development for treating leukemia, LY333′531, is a selective PIM-1 inhibitor. O. Fedorov, et al., PNAS 104(51), 20523-28 (December 2007). Evidence has been published to show that PIM-1 is involved in human tumors including prostate cancer, oral cancer, and Burkitt lymphoma (Gaidano & Dalla Faver, 1993). All these findings point to an important role of PIM-1 in the initiation and progression of human cancers, including various tumors and hematopoietic cancers, thus small molecule inhibitors of PIM-1 activity are a promising therapeutic strategy.

Additionally, PIM-2 and PIM-3 have overlapping functions with PIM-1 and inhibition of more than one isoform may provide additional therapeutic benefits. However, it is sometimes preferable for inhibitors of PIM to have little or no in vivo impact through their inhibition of various other kinases, since such effects are likely to cause side effects or unpredictable results. See, e.g., O. Fedorov, et al., PNAS 104(51), 20523-28 (December 2007), discussing the effects that non-specific kinase inhibitors can produce. Accordingly, in some embodiments, the invention provides compounds that are selective inhibitors of at least one of PIM-1, PIM-2, and PIM-3, or some combination of these, while having substantially less activity on certain other human kinases, as described further herein.

The implication of a role for PIM-3 in cancer was first suggested by transcriptional profiling experiments showing that PIM3 gene transcription was upregulated in EWS/ETS-induced malignant transformation of NIH 3T3 cells. These results were extended to show that PIM-3 is selectively expressed in human and mouse hepatocellular and pancreatic carcinomas but not in normal liver or pancreatic tissues. In addition, PIM-3 mRNA and protein are constitutively expressed in multiple human pancreatic and hepatocellular cancer cell lines.

The link between PIM-3 overexpression and a functional role in promoting tumorigenesis came from RNAi studies in human pancreatic and hepatocellular cancer cell lines overexpressing PIM-3. In these studies the ablation of endogenous PIM-3 protein promoted apoptosis of these cells. The molecular mechanism by which PIM-3 suppresses apoptosis is in part carried out through the modulation of phosphorylation of the pro-apoptotic protein BAD. Similar to both PIM-1 and 2 which phosphorylate BAD protein, the knockdown of PIM-3 protein by siRNA results in a decrease in BAD phosphorylation at SerII2. Thus, similar to PIM-1 and 2, PIM-3 acts a suppressor of apoptosis in cancers of endodermal origin, e.g., pancreatic and liver cancers. Moreover, as conventional therapies in pancreatic cancer have a poor clinical outcome, PIM-3 could represent a new important molecular target towards successful control of this incurable disease.

At the 2008 AACR Annual Meeting, SuperGen announced that it has identified a lead PIM kinase inhibitor, SGI-1776, that causes tumor regression in acute myelogenous leukemia (AML) xenograft models (Abstract No. 4974). In an oral presentation entitled, “A potent small molecule PIM kinase inhibitor with activity in cell lines from hematological and solid malignancies,” Dr. Steven Warner detailed how scientists used SuperGen's CLIMB™ technology to build a model that allowed for the creation of small molecule PIM kinase inhibitors. SGI-1776 was identified as a potent and selective inhibitor of the PIM kinases, inducing apoptosis and cell cycle arrest, thereby causing a reduction in phospho-BAD levels and enhancement of mTOR inhibition in vitro. Most notably, SGI-1776 induced significant tumor regression in MV-4-11 (AML) and MOLM-13 (AML) xenograft models. This demonstrates that inhibitors of PIM kinases can be used to treat leukemias.

Fedorov, et al., in PNAS vol. 104(51), 20523-28, showed that a selective inhibitor of PIM-1 kinase (Ly5333′531) suppressed cell growth and induced cell death in leukemic cells from AML patients. PIM-3 has been shown to be expressed in pancreatic cancer cells, while it is not expressed in normal pancreas cells, demonstrating that it should be a good target for pancreatic cancer. Li, et al., Cancer Res. 66(13), 6741-47 (2006) Inhibitors of PIM kinases that are described as useful for treating certain types of cancers are described in PCT/US2008/012829.

Because these protein kinases have important functions in biochemical pathways associated with cancer and inflammation, and are also important in pathogenicity of many microorganisms, inhibitors of their activity have many medicinal applications. The present invention provides novel compounds that inhibit CK2 or PIM or both, as well as compositions and methods of use utilizing these compounds.

### DISCLOSURE OF THE INVENTION

The present invention in part provides chemical compounds having certain biological activities that include, but are not limited to, inhibiting cell proliferation, inhibiting angiogenesis, and modulating protein kinase activities. These molecules can modulate casein kinase 2 (CK2) activity and/or Pim kinase activity, and thus affect biological functions that include but are not limited to, inhibiting gamma phosphate transfer from ATP to a protein or peptide substrate, inhibiting angiogenesis, inhibiting cell proliferation and inducing cell apoptosis, for example. The present invention also in part provides methods for preparing novel chemical compounds, and analogs thereof, and methods of using these compounds. Also provided are compositions comprising the above-described molecules in combination with other materials, including other therapeutic agents, and methods for using such compositions.

In one aspect, the invention provides compounds of Formula (I):

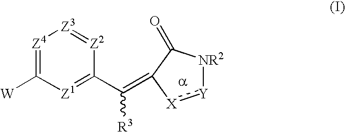

or a pharmaceutically acceptable salt thereof, wherein:

each of Z¹, Z², Z³ and Z⁴ is independently CR¹ or N, provided no more than three of Z¹, Z², Z³ and Z⁴ is N;

each R¹ is independently H, halo, CN, optionally substituted C1-C4 alkyl, optionally substituted C2-C4 alkenyl, optionally substituted C2-C4 alkynyl, optionally substituted C1-C4 alkoxy, or —NR⁶R⁷,
where R⁶ and R⁷ are independently selected from the group consisting of H, optionally substituted C1-C10 alkyl, optionally substituted aryl, optionally substituted arylalkyl, optionally substituted heteroaryl, and optionally substituted heteroarylalkyl, or
R⁶ and R⁷ taken together with the N in —NR⁶R⁷ can form an optionally substituted 5-8 membered ring that optionally contains an additional heteroatom selected from N, O and S as a ring member;

R² is H or C1-C4 alkyl;

R³ is H or optionally substituted C1-C10 alkyl;

α is a single bond,
X is O, S, or NR⁴, where R⁴ is H or an optionally substituted group selected from C1-C4 alkyl, C2-C4 alkenyl, and C2-C4 alkynyl, and
Y is C=Q, where Q is O or S; or

α is a double bond,
X is CR⁵, where R⁵ is H or an optionally substituted group selected from C1-C4 alkyl, C2-C4 alkenyl, and C2-C4 alkynyl, and
Y is N;

W is optionally substituted aryl or optionally substituted heteroaryl, or is —NR¹⁰NR¹¹,
wherein said aryl or heteroaryl group may be optionally substituted with a substituent selected from the group consisting of halo, C1-C4 alkyl, C1-C4 alkoxy, CN, —COOR⁸, —CONR⁸R⁹, —CONR⁸NR⁸R⁹, —SO₂NR⁸R⁹, —NR⁸R⁹, —NR⁸COR⁸, and —NR⁸CONR⁸R⁹,
where each R⁸, R⁹, R¹⁰ and R¹¹ is independently selected from the group consisting of H, optionally substituted C1-C10 alkyl, optionally substituted aryl, optionally substituted arylalkyl, optionally substituted heteroaryl, and optionally substituted heteroarylalkyl; or

R⁸ and R⁹ taken together with the N in NR⁸R⁹, and R¹⁰ and R¹¹ taken together with the N in NR¹⁰R¹¹ can independently form an optionally substituted 5-8 membered ring that optionally contains an additional heteroatom selected from N, O and S as a ring member;

provided that when W is phenyl, said phenyl is substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹,
and further provided that when said substituent on phenyl is —SO₂NR⁸R⁹, both of R⁸ and R⁹ are not H.

In another aspect, the invention provides compounds of Formula (II):

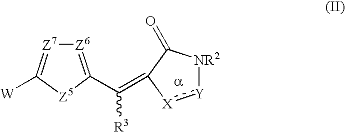

or a pharmaceutically acceptable salt thereof, wherein:

Z⁵ is O, S, or NR²¹, where R²¹ is H or optionally substituted C1-C10 alkyl;

each of Z⁶ and Z⁷ is independently CR¹ or N;

each R¹ is independently H, halo, CN, optionally substituted C1-C4 alkyl, optionally substituted C2-C4 alkenyl, optionally substituted C2-C4 alkynyl, optionally substituted C1-C4 alkoxy, or —NR⁶R⁷,
where R⁶ and R⁷ are independently selected from the group consisting of H, optionally substituted C1-C10 alkyl, optionally substituted aryl, optionally substituted arylalkyl, optionally substituted heteroaryl, and optionally substituted heteroarylalkyl, or
R⁶ and R⁷ taken together with the N in —NR⁶R⁷ can form an optionally substituted 5-8 membered ring that optionally contains an additional heteroatom selected from N, O and S as a ring member;

R² is H or C1-C4 alkyl;

R³ is H or optionally substituted C1-C10 alkyl;

α is a single bond,
X is O, S, or NR⁴, where R⁴ is H or an optionally substituted group selected from C1-C4 alkyl, C2-C4 alkenyl, and C2-C4 alkynyl, and
Y is C=Q, where Q is O or S; or

α is a double bond,
X is CR⁵, where R⁵ is H or an optionally substituted group selected from C1-C4 alkyl, C2-C4 alkenyl, and C2-C4 alkynyl, and
Y is N;

W is optionally substituted aryl or optionally substituted heteroaryl, or is —NR¹⁰NR¹¹,
wherein said aryl or heteroaryl group may be optionally substituted with a substituent selected from the group consisting of halo, C1-C4 alkyl, C1-C4 alkoxy, CN, —COOR^{S}, —CONR⁸R⁹, —CONR⁸NR⁸R⁹, —SO₂NR⁸R⁹, —NR⁸R⁹, —NR⁸COR⁸, and —NR⁸CONR⁸R⁹,
where each R⁸, R⁹, R¹⁰ and R¹¹ is independently selected from the group consisting of H, optionally substituted C1-C10 alkyl, optionally substituted aryl, optionally substituted arylalkyl, optionally substituted heteroaryl, and optionally substituted heteroarylalkyl; or
R⁸ and R⁹ taken together with the N in NR⁸R⁹, and R¹⁰ and R¹¹ taken together with the N in NR¹⁰R¹¹ can independently form an optionally substituted 5-8 membered ring that optionally contains an additional heteroatom selected from N, O and S as a ring member;
provided that when W is phenyl, said phenyl is substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹,
and further provided that when said substituent on phenyl is —SO₂NR⁸R⁹, both of R⁸ and R⁹ are not H.

In a further aspect, the invention provides compounds of Formula III:

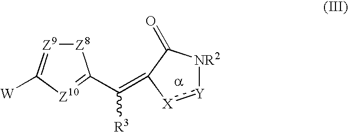

or a pharmaceutically acceptable salt thereof, wherein:

Z⁸ is O, S, or NR³¹, where R³¹ is H or optionally substituted C1-C10 alkyl;

each of Z⁹ and Z¹⁰ is independently CR¹ or N; each R¹ is independently H, halo, CN, optionally substituted C1-C4 alkyl, optionally substituted C2-C4 alkenyl, optionally substituted C2-C4 alkynyl, optionally substituted C1-C4 alkoxy, or —NR⁶R⁷,
where R⁶ and R⁷ are independently selected from the group consisting of H, optionally substituted C1-C10 alkyl, optionally substituted aryl, optionally substituted arylalkyl, optionally substituted heteroaryl, and optionally substituted heteroarylalkyl, or
R⁶ and R⁷ taken together with the N in —NR⁶R⁷ can form an optionally substituted 5-8 membered ring that optionally contains an additional heteroatom selected from N, O and S as a ring member;

R² is H or C1-C4 alkyl;

R³ is H or optionally substituted C1-C10 alkyl;

α is a single bond,
X is O, S, or NR⁴, where R⁴ is H or an optionally substituted group selected from C1-C4 alkyl, C2-C4 alkenyl, and C2-C4 alkynyl, and
Y is C=Q, where Q is O or S; or

α is a double bond,
X is CR⁵, where R⁵ is H or an optionally substituted group selected from C1-C4 alkyl, C2-C4 alkenyl, and C2-C4 alkynyl, and
Y is N;

W is optionally substituted aryl or optionally substituted heteroaryl, or is —NR¹⁰NR¹¹,
wherein said aryl or heteroaryl group may be optionally substituted with a substituent selected from the group consisting of halo, C1-C4 alkyl, C1-C4 alkoxy, CN, —COOR⁸, —CONR⁸R⁹, —CONR⁸NR⁸R⁹, —SO₂NR⁸R⁹, —NR⁸R⁹, —NR⁸COR⁸, and —NR⁸CONR⁸R⁹,
where each R⁸, R⁹, R¹⁰ and R¹¹ is independently selected from the group consisting of H, optionally substituted C1-C10 alkyl, optionally substituted aryl, optionally substituted arylalkyl, optionally substituted heteroaryl, and optionally substituted heteroarylalkyl; or
R⁸ and R⁹ taken together with the N in NR⁸R⁹, and R¹⁰ and R¹¹ taken together with the N in NR¹⁰R¹¹ can independently form an optionally substituted 5-8 membered ring that optionally contains an additional heteroatom selected from N, O and S as a ring member;
provided that when W is phenyl, said phenyl is substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹,
and further provided that when said substituent on phenyl is —SO₂NR⁸R⁹, both of R⁸ and R⁹ are not H.

The invention also includes the pharmaceutically acceptable salts of compounds of Formula I, II and III.

The invention also provides pharmaceutical compositions containing such compounds plus one or more pharmaceutically acceptable carriers or excipients, and methods of using these compounds and compositions for the treatment of specified conditions as further described herein.

Also provided herein are pharmaceutical compositions, comprising a compound of any of the formulae provided herein and at least one pharmaceutically acceptable carrier or excipient. Pharmaceutical compositions may contain two or more pharmaceutically acceptable carriers and/or excipients.

It is understood that the compounds of Formula I described herein include compounds of Formulae I-A, I-B, Ia, Ib, Ic, Id, Ie and If; that the compounds of Formula II described herein include compounds of Formulae II-A, II-B, IIa, IIb, IIe, IId, IIe, and that the compounds of Formula III described herein include compounds of Formulae III-A, III-B, IIIa, IIIb, IIIc, IIId, IIIe, and IIIf. Pharmaceutical compositions comprising at least one of these compounds can be utilized in methods of treatment such as those described herein.

The compounds of Formula I, II and III bind to certain kinase proteins, which are believed to be the basis for their pharmaceutical activity. In certain embodiments, the protein is a CK2 protein, such as a CK2 protein comprising the amino acid sequence of SEQ ID NO: 1, 2 or 3 or a substantially identical variant thereof, for example.

| SEQ ID NO: 1 (NP_001886; casein kinase II alpha 1 subunit | |
|---|---|
| isoform a [Homo sapiens]) | |
| | msgpvpsrar vytdvnthrp reywdyeshv vewgnqddyq lvrklgrgky sevfeainit |
| | nnekvvvkil kpvkkkkikr eikilenlrg gpniitladi vkdpvsrtpa lvfehvnntd |
| 121 | fkqlyqtltd ydirfymyei lkaldychsm gimhrdvkph nvmidhehrk lrlidwglae |
| 181 | fyhpgqeynv rvasryfkgp ellvdyqmyd ysldmwslgc mlasmifrke pffhghdnyd |
| 241 | qlvriakvlg tedlydyidk ynieldprfn dilgrhsrkr werfvhsenq hlvspealdf |
| 301 | ldkllrydhq srltareame hpyfytvvkd qarmgsssmp ggstpvssan mmsgissvpt |
| 361 | psplgplags pviaaanplg mpvpaaagaq q |
| SEQ ID NO: 2 (NP_808227; casein kinase II alpha 1 subunit | |
| isoform a [Homo sapiens]) | |
| | msgpvpsrar vytdvnthrp reywdyeshv vewgnqddyq lvrklgrgky sevfeainit |
| | nnekvvvkil kpvkkkkikr eikilenlrg gpniitladi vkdpvsrtpa lvfehvnntd |
| 121 | fkqlyqtltd ydirfymyei lkaldychsm gimhrdvkph nvmidhehrk lrlidwglae |
| 181 | fyhpgqeynv rvasryfkgp ellvdyqmyd ysldmwslgc mlasmifrke pffhghdnyd |
| 241 | qlvriakvlg tedlydyidk ynieldprfn dilgrhsrkr werfvhsenq hlvspealdf |
| 301 | ldkllrydhq srltareame hpyfytvvkd qarmgsssmp ggstpvssan mmsgissvpt |
| 361 | psplgplags pviaaanplg mpvpaaagaq q |
| SEQ ID NO: 3 (NP_808228; casein kinase II alpha 1 subunit | |
| isoform b [Homo sapiens]) | |
| | myeilkaldy chsmgimhrd vkphnvmidh ehrklrlidw glaefyhpgq eynvrvasry |
| | fkgpellvdy qmydysldmw slgcmlasmi frkepffhgh dnydqlvria kvlgtedlyd |
| 121 | yidkynield prfndilgrh srkrwerfvh senqhlvspe aldfldkllr ydhqsrltar |
| 181 | eamehpyfyt vvkdqarmgs ssmpggstpv ssanmmsgis svptpsplgp lagspviaaa |
| 241 | nplgmpvpaa agaqq |

Substantially identical variants of these include proteins having at least 90% sequence homology with one of these, preferably at least 90% sequence identity; and having at least 50% of the level of in vitro kinase activity of the specified sequence.

The invention includes methods to modulate the activity of CK2 protein, either in vitro or ex vivo. Suitable methods comprise contacting a system comprising the protein with a compound described herein in an amount effective for modulating the activity of the protein. In certain embodiments the activity of the protein is inhibited, and sometimes the protein is a CK2 protein comprising the amino acid sequence of SEQ ID NO: 1, 2 or 3 or a substantially identical variant thereof, for example. In certain embodiments the CK2 is in a cell or tissue; in other embodiments, it can be in a cell-free system.

Also provided are methods for modulating the activity of a Pim protein, which comprise contacting a system comprising the protein with a compound described herein in an amount effective for modulating the activity of the protein. In certain embodiments, the system is a cell, and in other embodiments the system is a cell-free system. In certain embodiments, the activity of the Pim protein is inhibited.

Provided also are methods for inhibiting cell proliferation, which comprise contacting cells with a compound of any of the formulae described herein in an amount effective to inhibit proliferation of the cells. The cells sometimes are in a cell line, such as a cancer cell line (e.g., breast cancer, prostate cancer, pancreatic cancer, lung cancer, hemopoietic cancer, colorectal cancer, skin cancer, ovary cancer cell line), for example. In some embodiments, the cancer cell line is a breast cancer, prostate cancer or pancreatic cancer cell line. The cells sometimes are in a tissue, can be in a subject, at times are in a tumor, and sometimes are in a tumor in a subject. In certain embodiments, the method further comprises inducing cell apoptosis. Cells sometimes are from a subject having macular degeneration.

Also provided are methods for treating a condition related to aberrant cell proliferation, which comprise administering a compound of one of the formulae described herein to a subject in need thereof in an amount effective to treat the cell proliferative condition. In certain embodiments the cell proliferative condition is a tumor-associated cancer. The cancer sometimes is cancer of the breast, prostate, pancreas, lung, colorectum, skin, or ovary. In some embodiments, the cell proliferative condition is a non-tumor cancer, such as a hematopoietic cancer, for example, including leukemias and lymphomas. The cell proliferative condition is macular degeneration in some embodiments.

The invention also includes methods for treating cancer or an inflammatory disorder in a subject in need of such treatment, comprising: administering to the subject a therapeutically effective amount of a therapeutic agent useful for treating such disorder; and administering to the subject a molecule that inhibits CK2 and/or Pim in an amount that is effective to enhance a desired effect of the therapeutic agent. In certain embodiments, the molecule that inhibits CK2 and/or Pim is a compound of Formula I, II or III, or a pharmaceutically acceptable salt thereof. In certain embodiments, the desired effect of the therapeutic agent that is enhanced by the molecule that inhibits CK2 and/or Pim is an increase in apoptosis in at least one type of cell.

In some embodiments, the therapeutic agent and the molecule that inhibits CK2 and/or Pim are administered at substantially the same time. The therapeutic agent and molecule that inhibits CK2 and/or Pim sometimes are used concurrently by the subject. The therapeutic agent and the molecule that inhibits CK2 and/or Pim can be combined into one pharmaceutical composition in certain embodiments; in other embodiments they are administered as separate compositions.

Also provided are compositions of matter comprising a compound of one of the formulae described herein and an isolated protein. The protein sometimes is a CK2 protein, such as a CK2 protein comprising the amino acid sequence of SEQ ID NO: 1, 2 or 3 or a substantially identical variant thereof, for example. In some embodiments, the protein is a Pim protein. Certain compositions comprise a compound described herein in combination with a cell. The cell may be from a cell line, such as a cancer cell line. In the latter embodiments, the cancer cell line is sometimes a breast cancer, prostate cancer, pancreatic cancer, lung cancer, hematopoietic cancer, colorectal cancer, skin cancer, of ovary cancer cell line.

These and other embodiments of the invention are described in the description that follows.

### MODES OF CARRYING OUT THE INVENTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular forms “a”, “an”, and “the” include plural references unless indicated otherwise.

Compounds of the formulae provided herein exert biological activities that include, but are not limited to, inhibiting cell proliferation. Compounds of these Formulae can modulate CK2 activity, Pim activity or both, as demonstrated by the data herein. Such compounds therefore can be utilized in multiple applications by a person of ordinary skill in the art. For example, compounds described herein may find uses that include, but are not limited to, (i) modulation of protein kinase activity (e.g., CK2 activity), (ii) modulation of Pim activity (e.g., PIM-1 activity), (iii) modulation of cell proliferation, (iv) modulation of apoptosis, and (v) treatments of cell proliferation related disorders (e.g., administration alone or co-administration with another molecule).

In some cases, the compounds of the invention contain one or more chiral centers. The invention includes each of the isolated stereoisomeric forms as well as mixtures of stereoisomers in varying degrees of chiral purity, including racemic mixtures. It also encompasses the various diastereomers and tautomers that can be formed, including both E and Z isomers of double bonds that are not in rings. The compounds of the invention may also exist in more than one tautomeric form; the depiction herein of one tautomer is for convenience only, and is also understood to encompass other tautomers of the form shown.

As an example, only, the compounds of Formula I, II and III have a Carbon-Carbon double bond to which group R³ is attached. The Formulae are depicted to indicate it can represent either the E isomer or the Z isomer, or both. Other structures may appear to depict a specific isomer, but that is merely for convenience, and is not intended to limit the invention to the depicted olefin isomer.

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10C or as C1-C10 or C1-C10. When heteroatoms (N, O and S typically) are allowed to replace carbon atoms as in heteroalkyl groups, for example, the numbers describing the group, though still written as e.g. C1-C6, represent the sum of the number of carbon atoms in the group plus the number of such heteroatoms that are included as replacements for carbon atoms in the backbone of the ring or chain being described.

Typically, the alkyl, alkenyl and alkynyl substituents of the invention contain 1-10C (alkyl) or 2-10C (alkenyl or alkynyl). Preferably they contain 1-8C (alkyl) or 2-8C (alkenyl or alkynyl). Sometimes they contain 1-4C (alkyl) or 2-4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often optionally substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCSNR₂, NRC(═NR)NR₂, NRCOOR, NRCOR, CN, CCR, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C3-C8 heterocyclyl, C4-C10 heterocyclylalkyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂, wherein each R¹ is independently H, C1-C8 alkyl, C2-C8 heteroalkyl, C1-C8 acyl, C3-C8 heterocyclyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl, C3-C8 cycloalkyl, C3-C8 heterocyclyl, or C5-C10 heteroaryl, each of which can be substituted by the substituents that are appropriate for the particular group. Where a substituent group contains two R or R′ groups on the same or adjacent atoms (e.g., —NR₂, or —NR—C(O)R), the two R or R′ groups can optionally be taken together with the atoms in the substituent group to which they are attached to form a ring having 5-8 ring members, which can be substituted as allowed for the R or R′ itself, and can contain an additional heteroatom (N, O or S) as a ring member.

“Optionally substituted” as used herein indicates that the particular group or groups being described may have no non-hydrogen substituents, or the group or groups may have one or more non-hydrogen substituents. If not otherwise specified, the total number of such substituents that may be present is equal to the number of H atoms present on the unsubstituted form of the group being described. Where an optional substituent is attached via a double bond, such as a carbonyl oxygen (═O), the group takes up two available valences, so the total number of substituents that may be included is reduced according to the number of available valences.

“Acetylene” substituents are 2-10C alkynyl groups that are optionally substituted, and are of the formula —C≡C—R^{a}, wherein R^{a} is H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

and each R^{a} group is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂, wherein each R¹ is independently H, C1-C6 alkyl, C2-C6 heteroalkyl, C1-C6 acyl, C2-C6 heteroacyl, C6-C10 aryl, C5-C10 heteroaryl, C7-C12 arylalkyl, or C6-C12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O; and wherein two R′ can be linked to form a 3-7 membered ring optionally containing up to three heteroatoms selected from N, O and S. In some embodiments, R^{a} of —C≡C—R^{a} is H or Me.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical and preferred sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and ═C(═O)NR₂ as well as ═C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably the monocyclic heteroaryls contain 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCSNR₂, NRC(═NR)NR₂, NRCOOR, NRCOR, CN, CCR, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C3-C8 heterocyclyl, C4-C10 heterocyclylalkyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups. Where a substituent group contains two R or R′ groups on the same or adjacent atoms (e.g., —NR2, or —NR—C(O)R), the two R or R′ groups can optionally be taken together with the atoms in the substituent group to which the are attached to form a ring having 5-8 ring members, which can be substituted as allowed for the R or R′ itself, and can contain an additional heteroatom (N, O or S) as a ring member.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C1-C4 alkylene that is unsubstituted or is substituted with one or two C1-C4 alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. Thus —CH(Me)- and ═C(Me)₂- may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Heteroform” as used herein refers to a derivative of a group such as an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Halo”, as used herein includes fluoro, chloro, bromo and iodo. Fluoro and chloro are often preferred.

“Amino” as used herein refers to NH₂, but where an amino is described as “substituted” or “optionally substituted”, the term includes NR′R′ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R′ is an aromatic group, it is optionally substituted with the substituents described as typical for heteroaryl groups.

As used herein, the term “carbocycle” or “carbocyclic” refers to a cyclic ring containing only carbon atoms in the ring, whereas the term “heterocycle” or “heterocyclic” refers to a ring comprising a heteroatom. The carbocyclic and heterocyclic structures encompass compounds having monocyclic, bicyclic or multiple ring systems.

As used herein, the term “heteroatom” refers to any atom that is not carbon or hydrogen, such as nitrogen, oxygen or sulfur. When it is part of the backbone or skeleton of a chain or ring, a hetero atom must be at least divalent, and will typically be selected from N, O, P, and S.

Illustrative examples of heterocycles include but are not limited to tetrahydrofuran, 1,3-dioxolane, 2,3-dihydrofuran, pyran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4b]pyridine, piperazine, piperidine, homopiperazine, homopiperidine, pyrazine, morpholine, thiomorpholine, homomorpholine, homothiomorpholine, imidazole, imidazolidine 2,4-dione, 1,3-dihydrobenzimidazol-2-one, indole, thiazole, benzothiazole, thiadiazole, thiophene, tetrahydro thiophene 1,1-dioxide, diazepine, triazole, guanidine, diazabicyclo[2.2.1]heptane, 2,5-diazabicyclo[2.2.1]heptane, 2,3,4,4a,9,9a-hexahydro-1H-β-carboline, oxirane, oxetane, tetrahydropyran, dioxane, lactones, aziridine, azetidine, piperidine, lactams, and may also encompass heteroaryls. Other illustrative examples of heteroaryls include but are not limited to furan, thiophene, pyrrole, imidazole, oxazole, thiazole, oxadiazole, thiadiazole, pyridine, pyrimidine, pyridazine, pyrazine, benzimidazole and triazole.

In one aspect, the invention provides compounds of Formula I:

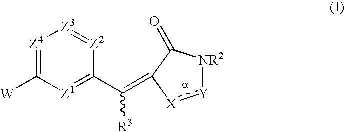

or a pharmaceutically acceptable salt thereof, wherein:

each of Z¹, Z², Z³ and Z⁴ is independently CR¹ or N, provided no more than three of Z¹, Z², Z³ and Z⁴ is N;

each R¹ is independently H, halo, CN, optionally substituted C1-C4 alkyl, optionally substituted C2-C4 alkenyl, optionally substituted C2-C4 alkynyl, optionally substituted C1-C4 alkoxy, or —NR⁶R⁷,
where R⁶ and R⁷ are independently selected from the group consisting of H, optionally substituted C1-C10 alkyl, optionally substituted aryl, optionally substituted arylalkyl, optionally substituted heteroaryl, and optionally substituted heteroarylalkyl, or
R⁶ and R⁷ taken together with the N in —NR⁶R⁷ can form an optionally substituted 5-8 membered ring that optionally contains an additional heteroatom selected from N, O and S as a ring member;

R² is H or C1-C4 alkyl;

R³ is H or optionally substituted C1-C10 alkyl;

α is a single bond,
X is O, S, or NR⁴, where R⁴ is H or an optionally substituted group selected from C1-C4 alkyl, C2-C4 alkenyl, and C2-C4 alkynyl, and
Y is C=Q, where Q is O or S; or

α is a double bond,
X is CR⁵, where R⁵ is H or an optionally substituted group selected from C1-C4 alkyl, C2-C4 alkenyl, and C2-C4 alkynyl, and
Y is N;

W is optionally substituted aryl or optionally substituted heteroaryl, or is —NR¹⁰R¹¹,
wherein said aryl or heteroaryl group may be optionally substituted with a substituent selected from the group consisting of halo, C1-C4 alkyl, C1-C4 alkoxy, CN, —COOR⁸, —CONR⁸R⁹, —CONR⁸NR⁸R⁹, —SO₂NR⁸R⁹, —NR⁸R⁹, —NR⁸COR⁸, and —NR⁸CONR⁸R⁹,
where each R⁸, R⁹, R¹⁰ and R¹¹ is independently selected from the group consisting of H, optionally substituted C1-C10 alkyl, optionally substituted aryl, optionally substituted arylalkyl, optionally substituted heteroaryl, and optionally substituted heteroarylalkyl; or
R⁸ and R⁹ taken together with the N in NR⁸R⁹, and R¹⁰ and R¹¹ taken together with the N in NR¹⁰R¹¹ can independently form an optionally substituted 5-8 membered ring that optionally contains an additional heteroatom selected from N, O and S as a ring member;
provided that when W is phenyl, said phenyl is substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹,
and further provided that when said substituent on phenyl is —SO₂NR⁸R⁹, both of R⁸ and R⁹ are not H.

In another aspect, the invention provides compounds of Formula II:

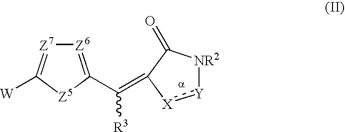

or a pharmaceutically acceptable salt thereof, wherein:

Z⁵ is O, S, or NR²¹, where R²¹ is H or optionally substituted C1-C10 alkyl;

each of Z⁶ and Z⁷ is independently CR¹ or N;

each R¹ is independently H, halo, CN, optionally substituted C1-C4 alkyl, optionally substituted C2-C4 alkenyl, optionally substituted C2-C4 alkynyl, optionally substituted C1-C4 alkoxy, or —NR⁶R⁷,
where R⁶ and R⁷ are independently selected from the group consisting of H, optionally substituted C1-C10 alkyl, optionally substituted aryl, optionally substituted arylalkyl, optionally substituted heteroaryl, and optionally substituted heteroarylalkyl, or
R⁶ and R⁷ taken together with the N in —NR⁶R⁷ can form an optionally substituted 5-8 membered ring that optionally contains an additional heteroatom selected from N, O and S as a ring member;

R² is H or C1-C4 alkyl;

R³ is H or optionally substituted C1-C10 alkyl;

α is a single bond,
X is O, S, or NR⁴, where R⁴ is H or an optionally substituted group selected from C1-C4 alkyl, C2-C4 alkenyl, and C2-C4 alkynyl, and
Y is C=Q, where Q is O or S; or

α is a double bond,
X is CR⁵, where R⁵ is H or an optionally substituted group selected from C1-C4 alkyl, C2-C4 alkenyl, and C2-C4 alkynyl, and
Y is N;

W is optionally substituted aryl or optionally substituted heteroaryl, or is —NR¹⁰NR¹¹,
wherein said aryl or heteroaryl group may be optionally substituted with a substituent selected from the group consisting of halo, C1-C4 alkyl, C1-C4 alkoxy, CN, —COOR⁸, —CONR⁸R⁹, —CONR⁸NR⁸R⁹, —SO₂NR⁸R⁹, —NR⁸R⁹, —NR⁸COR⁸, and —NR⁸CONR⁸R⁹,
where each R⁸, R⁹, R¹⁰ and R¹¹ is independently selected from the group consisting of H, optionally substituted C1-C10 alkyl, optionally substituted aryl, optionally substituted arylalkyl, optionally substituted heteroaryl, and optionally substituted heteroarylalkyl; or
R⁸ and R⁹ taken together with the N in NR⁸R⁹, and R¹⁰ and R¹¹ taken together with the N in NR¹⁰R¹¹ can independently form an optionally substituted 5-8 membered ring that optionally contains an additional heteroatom selected from N, O and S as a ring member;
provided that when W is phenyl, said phenyl is substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹,
and further provided that when said substituent on phenyl is —SO₂NR⁸R⁹, both of R⁸ and R⁹ are not H.

In a further aspect, the invention provides compounds of Formula III:

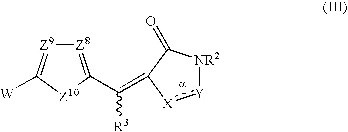

or a pharmaceutically acceptable salt thereof, wherein:

Z⁸ is O, S, or NR³¹, where R³¹ is H or optionally substituted C1-C10 alkyl;

each of Z⁹ and Z¹⁰ is independently CR¹ or N;

each R¹ is independently H, halo, CN, optionally substituted C1-C4 alkyl, optionally substituted C2-C4 alkenyl, optionally substituted C2-C4 alkynyl, optionally substituted C1-C4 alkoxy, or —NR⁶R⁷,
where R⁶ and R⁷ are independently selected from the group consisting of H, optionally substituted C1-C10 alkyl, optionally substituted aryl, optionally substituted arylalkyl, optionally substituted heteroaryl, and optionally substituted heteroarylalkyl, or
R⁶ and R⁷ taken together with the N in —NR⁶R⁷ can form an optionally substituted 5-8 membered ring that optionally contains an additional heteroatom selected from N, O and S as a ring member;

R² is H or C1-C4 alkyl;

R³ is H or optionally substituted C1-C10 alkyl;

α is a single bond,
X is O, S, or NR⁴, where R⁴ is H or an optionally substituted group selected from C1-C4 alkyl, C2-C4 alkenyl, and C2-C4 alkynyl, and
Y is C=Q, where Q is O or S; or

α is a double bond,
X is CR⁵, where R⁵ is H or an optionally substituted group selected from C1-C4 alkyl, C2-C4 alkenyl, and C2-C4 alkynyl, and
Y is N;

W is optionally substituted aryl or optionally substituted heteroaryl, or is —NR¹⁰NR¹¹,
wherein said aryl or heteroaryl group may be optionally substituted with a substituent selected from the group consisting of halo, C1-C4 alkyl, C1-C4 alkoxy, CN, —COOR⁸, —CONR⁸R⁹, —CONR⁸NR⁸R⁹, —SO₂NR⁸R⁹, —NR⁸R⁹, —NR⁸COR⁸, and —NR⁸CONR⁸R⁹,
where each R⁸, R⁹, R¹⁰ and R¹¹ is independently selected from the group consisting of H, optionally substituted C1-C10 alkyl, optionally substituted aryl, optionally substituted arylalkyl, optionally substituted heteroaryl, and optionally substituted heteroarylalkyl; or
R⁸ and R⁹ taken together with the N in NR⁸R⁹, and R¹⁰ and R¹¹ taken together with the N in NR¹⁰R¹¹ can independently form an optionally substituted 5-8 membered ring that optionally contains an additional heteroatom selected from N, O and S as a ring member;
provided that when W is phenyl, said phenyl is substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹,
and further provided that when said substituent on phenyl is —SO₂NR⁸R⁹, both of R⁸ and R⁹ are not H.

Compounds of Formulae I, II and III are characterized by a core aromatic or heteroaromatic ring, which is linked to an additional heterocyclic group via an sp2 carbon atom and is further substituted by a group, W, as further described herein. In frequent embodiments, the additional heterocyclic group and the group W are disposed on the core aromatic or heteroaromatic ring in a 1,3- or meta-orientation.

In compounds of Formula I, the core ring is an optionally substituted 6-membered aromatic or heteroaromatic ring containing 0, 1, 2 or 3 nitrogen atoms at positions Z¹, Z², Z³ and Z⁴. Each of Z¹, Z², Z³ and Z⁴ is independently CR¹ or N, where R¹ is as described herein, provided that no more than three of Z¹, Z², Z³ and Z⁴ is N. In certain embodiments, each of Z¹, Z², Z³ and Z⁴ is CR¹, i.e., the core ring is a phenyl ring. In other embodiments, one of Z¹, Z², Z³ and Z⁴ is N and the other three of Z¹, Z², Z³ and Z⁴ are CR¹ (i.e., the core ring is a pyridine ring). In further embodiments, two of Z¹, Z², Z³ and Z⁴ are N and the other two of Z¹, Z², Z³ and Z⁴ are CR¹ (i.e., the core ring is a pyrimidine, pyrazine or pyridazine ring). In certain embodiments of Formula I, each of Z¹, Z², Z³ and Z⁴ is CR¹, and each R¹ is H.

In compounds of Formula II and III, the core ring is an optionally substituted 5-membered heteroaromatic ring containing 1, 2 or 3 heteroatoms, as further described herein.

In compounds of Formula II, Z⁵ is O, S, or NR²¹, where R²¹ is H or optionally substituted C1-C10 alkyl. Preferably, R²¹ is H or Me. Each of Z⁶ and Z⁷ is independently CR¹ or N, where R¹ is as described herein. Preferably, R¹ is H. In certain embodiments of Formula II, Z⁵ is O and each of Z⁶ and Z⁷ is CR¹. In some such embodiments, each R¹ is H. In other embodiments, Z⁵ is S and each of Z⁶ and Z⁷ is CR¹. In still further embodiments, Z⁵ is O, S, or NR²¹, one of Z⁶ and Z⁷ is N and the other is CR¹.

In specific embodiments of Formula II, the core 5-membered heterocyclic ring is a furan, thiophene, pyrrole, imidazole, oxazole, thiazole, oxadiazole or thiadiazole ring, each of which may be optionally substituted by R¹ or R²¹; preferably, it is an optionally substituted furan or thiophene ring.

In compounds of Formula III, Z⁸ is O, S, or NR³¹, where R³¹ is H or optionally substituted C1-C10 alkyl. Preferably, R³¹ is H or Me. Each of Z⁹ and Z¹⁰ is independently CR¹ or N, where R¹ is as described herein. Preferably, R¹ is H. In certain embodiments of Formula III, Z⁸ is S and each of Z⁹ and Z¹⁰ is CR¹. In some such embodiment, each R¹ is H. In other embodiments, Z⁸ is O and each of Z⁹ and Z¹⁰ is CR¹. In still further embodiments, Z⁸ is O, S, or NR³¹, one of Z⁹ and Z¹⁰ is N and the other is CR¹.

In specific embodiments of Formula III, the core 5-membered heterocyclic ring is a furan, thiophene, pyrrole, imidazole, oxazole, thiazole, oxadiazole or thiadiazole ring, each of which may be optionally substituted by R¹ or R³¹; preferably, it is an optionally substituted furan or thiophene ring.

In compounds of Formula I, the 6-membered aromatic or heteroaromatic core ring may be substituted by one or more substituent groups R¹ when at least one of Z¹, Z², Z³ and Z⁴ is CR¹.

In compounds of Formula II, the 5-membered heteroaromatic core ring may be substituted by one or more substituent groups R′, when at least one of Z⁶ and Z⁷ is CR¹.

In compounds of Formula III, the 5-membered heteroaromatic core ring may be substituted by one or more substituent groups R′, when at least one of Z⁹ and Z¹⁰ is CR¹.

In compounds of Formula I, II and III, each R¹ is independently H, halo, CN, optionally substituted C1-C4 alkyl, optionally substituted C2-C4 alkenyl, optionally substituted C2-C4 alkynyl, optionally substituted C1-C4 alkoxy, or an amino group, —NR⁶R⁷.

When R¹ is —NR⁶R⁷, R⁶ and R⁷ are independently selected from the group consisting of H, optionally substituted C1-C10 alkyl, optionally substituted aryl, optionally substituted arylalkyl, optionally substituted heteroaryl, and optionally substituted heteroarylalkyl, or R⁶ and R⁷ taken together with the N in —NR⁶R⁷ can form an optionally substituted 5-8 membered ring that optionally contains an additional heteroatom selected from N, O and S as a ring member.

In some embodiments, each R¹ is independently H, halo or an optionally substituted alkyl, amine or alkoxy group. In some embodiments, R¹ is H, halo, or a small alkyl, such as Me, Et, CF₃, —CH₂OMe, vinyl, or acetylene. In certain embodiments, R¹ is H, halo, Me, OMe, CF₃. In frequent embodiments, each R¹ is H.

Compounds of Formula I, II and III contain an additional heterocyclic group linked to the core 6-membered aromatic or 5-6 membered heteroaromatic ring. The additional heterocyclic group contains an amide linkage within the ring. In frequent embodiments, the additional heterocyclic group contains an amide linkage within the ring plus an additional carbonyl or thiocarbonyl (C═O or C═S). In frequent embodiments, the additional heterocyclic group comprises a 2-thio-2,4-thiazolidinedione (i.e., rhodanine) ring, a 2,4-thiazolidinedione ring or a hydantoin ring. The additional heterocyclic group is linked to the core aromatic or heteroaromatic ring through an exocyclic methylene group (i.e., an sp² carbon).

In compounds of Formula I, II and III, the additional heterocyclic group contains a bond, α, that may be a single bond or a double dond. In some embodiments, α is a single bond, X can be O, S, or NR⁴, and Y is a carbonyl or thiocarbonyl group, represented as C═O or C═S. When X is NR⁴, R⁴ is H or an optionally substituted group selected from C1-C4 alkyl, C2-C4 alkenyl, and C2-C4 alkynyl. Preferably, NR⁴ is NH.

In compounds of formula I, when X is NR⁴ and the core ring is a phenyl ring (i.e., Z¹-Z⁴ are each CR¹), R⁴ is preferably not benzyl, or benzyl substituted by Me, CF₃, or OMe; in particular, R⁴ is preferably not benzyl or 3-trifluoromethylbenzyl. In some embodiments, X is O and Y is C═O. In other embodiments, X is O and Y is C═S. In certain embodiments, X is S, and Y is C═O. In further embodiments, X is S, and Y is C═S.

In other embodiments of Formula I, II and III, α is a double bond, X is CR⁵, and Y is N. In such embodiments, R⁵ is H or an optionally substituted group selected from C1-C4 alkyl, C2-C4 alkenyl, and C2-C4 alkynyl. Sometimes, R⁵ is H or methyl. Preferably, CR⁵ is CH.

The additional heterocyclic group also contains NR², and R² in this group can be H or C1-C4 alkyl, such as Me or Et; preferably, R² is H.

The sp² carbon connecting the 6-membered aromatic or heteroaromatic ring and the heterocyclic group is substituted with R³, where R³ can be H or optionally substituted C1-C10 alkyl. In some embodiments, R³ is H or C1-C4 alkyl, such as Me; in preferred embodiments, it is H.

In compounds of Formula I, II and III, the core 6-membered aromatic or 5-6 membered heteroaromatic ring is also substituted by a group, W, where W can present a range of different features while retaining the desired protein kinase modulatory activities. As described herein, the group W is preferably disposed on the core ring with a 1,3- or meta-orientation with respect to the linkage to the additional heterocyclic group.

In certain embodiments of Formula I, II and III, W is an optionally substituted aryl or heteroaryl group. In some such embodiments, W is selected from the group consisting of an optionally substituted phenyl, pyridine, pyrimidine, and pyrazine ring. In particular, W can be an optionally substituted phenyl or pyrazine ring.

When W is an aryl or heteroaryl group, it may be optionally substituted with at least one substituent selected from the group consisting of halo, C1-C4 alkyl, C1-C4 alkoxy, CN, —COOR⁸, —CONR⁸R⁹, —CONR⁸NR⁸R⁹, —SO₂NR⁸R⁹, —NR⁸R⁹, —NR⁸COR⁸, and —NR⁸CONR⁸R⁹, provided that when W is phenyl, it is substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹, and further provided that when the substituent on phenyl is —SO₂NR⁸R⁹, both of R⁸ and R⁹ are not H.

In some embodiments, each of R⁸ and R⁹ is independently selected from the group consisting of H, optionally substituted C1-C10 alkyl, optionally substituted aryl, optionally substituted arylalkyl, optionally substituted heteroaryl, and optionally substituted heteroarylalkyl.

In further embodiments, R⁸ and R⁹ taken together with the N in NR⁸R⁹ can independently form an optionally substituted 5-8 membered ring that optionally contains an additional heteroatom selected from N, O and S as a ring member. Exemplary rings include optionally substituted pyrrolidine, pyrrolidinone, piperidine, homopiperidine, piperazine, homopiperazine, morpholine, thiomorpholine, homomorpholine, homothiomorpholine, and the like. In some embodiments, such rings are substituted with a C1-C4 alkyl, C1-C4 alkoxy, C1-C4 hydroxylalkyl, halo, acyl, OH, —NR′R″, COOR′, or CONR′R″, where R′ and R″ are independently H or C1-C4 alkyl, or may be taken together with N to form an optionally substituted 5-8 membered ring optionally containing an additional heteroatoms selected from N, O an S.

In certain embodiments, W is phenyl, substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹, where R⁸ and R⁹ are as described above. In certain embodiments, the phenyl group may be further substituted by at least one additional group other than H, such as F, Cl, Me, CF₃, CN, OMe. In other embodiments, no additional substituents other than H are present. In specific embodiments, W is phenyl substituted with —CONR⁸R⁹ or —CONR⁸NR⁸R⁹, where R⁸ and R⁹ in N of NR⁸R⁹ are taken together to form an optionally substituted pyrrolidine, pyrrolidinone, piperidine, homopiperidine, piperazine, homopiperazine, morpholine, thiomorpholine, homomorpholine, or homothiomorpholine ring. In further embodiments, W is phenyl substituted with —CONR⁸R⁹ or —CONR⁸NR⁸R⁹, wherein at least one of R⁸ and R⁹ is C1-C6 alkyl or C1-C6 heteroalkyl group, optionally substituted with an optionally substituted carbocyclic or optionally substituted heterocyclic ring (which may be aromatic, saturated or partially unsaturated), OH, C1-C4 alkoxy, or —NR′R″, where R′ and R″ are independently H or C1-C4 alkyl or may be taken together with N to form an optionally substituted 5-8 membered ring optionally containing an additional heteroatoms selected from N, O an S.

In other embodiments, W is an optionally substituted pyrazine ring. In some such embodiments, the pyrazine ring is substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹, where R⁸ and R⁹ are as described above. In specific embodiments, W is pyrazine substituted with —NR⁸R⁹, where R⁸ and R⁹ are as described above. In some such embodiments, R⁸ and R⁹ in —NR⁸R⁹ may be taken together to form an optionally substituted 5-8 membered ring that optionally contains an additional heteroatom selected from N, O and S as a ring member, such as an optionally substituted pyrrolidine, pyrrolidinone, piperidine, homopiperidine, piperazine, homopiperazine, morpholine, thiomorpholine, homomorpholine, or homothiomorpholine ring. In a preferred embodiment, W is pyrazine substituted by the group —NR⁸R⁹, where R⁸ and R⁹ in —NR⁸R⁹ are taken together to form an optionally substituted homopiperazine ring.

In certain embodiments, the invention provides a compound of Formula I having the structure of Formula I-A, I-B, Ia or Ib:

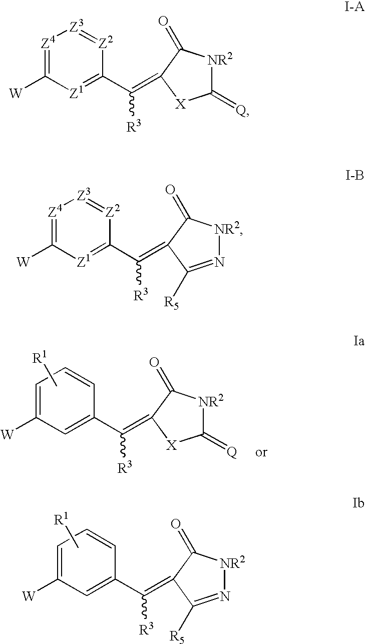

or a pharmaceutically acceptable salt thereof,

wherein Z¹, Z², Z³, Z⁴, R¹, R², R³, R⁵, W and X are defined as for Formula I; and

Q is O or S.

In certain preferred embodiments, the compound of Formula I has the structure of Formula Ic, Formula Id, Formula Ie or Formula If:

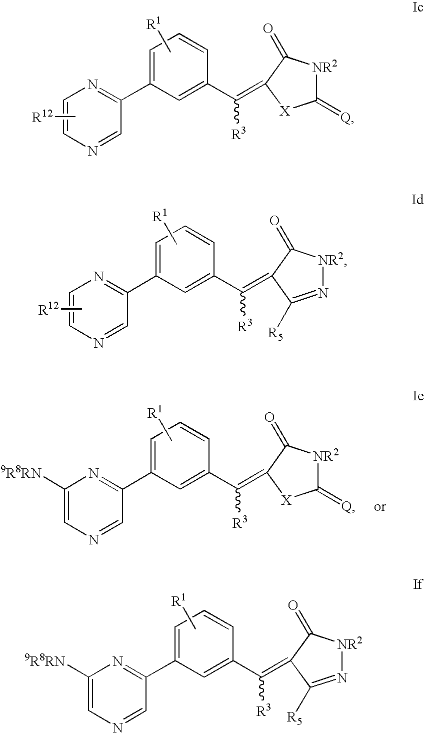

or a pharmaceutically acceptable salt thereof, wherein

R¹, R², R³, R⁵, R⁸, R⁹, and X are defined as for Formula I,

Q is O or S, and

R¹² is —CONR⁸R⁹, —CONR⁸R⁸R⁹, —SO₂NR⁸R⁹, —NR⁸R⁹, —NR⁸COR⁸, or —NR⁸CONR⁸R⁹.

In some embodiments of Formulae I-A, I-B, Ia, Ib, Ic, Id, Ie, and If, Ie and R³ are H. In other embodiments of Formulae I-A, I-B, Ia, Ib, Ic, Id, Ie, and If, R² is H. In preferred embodiments of Formulae I-A, I-B, Ia, Ib, Ic, Id, Ie, and If, each of Ie, R² and R³ is H.

In other embodiments, the invention provides a compound of Formula II having the structure of Formula II-A, II-B, IIa or IIb:

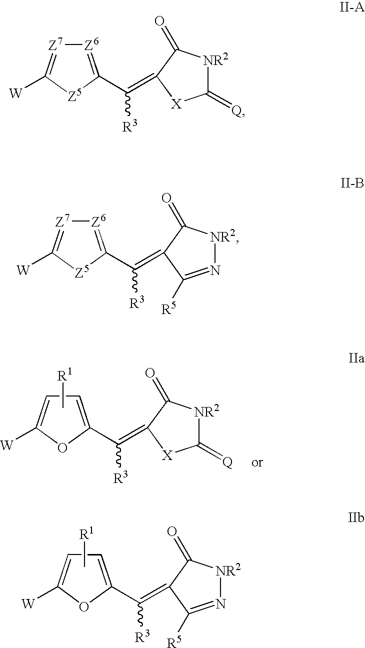

or a pharmaceutically acceptable salt thereof,

wherein Z⁵, Z⁶, Z⁷, R¹, R², R³, R⁵, W, and X are as defined in Formula II; and

Q is O or S.

In certain preferred embodiments, the compound of Formula II has the structure of Formula IIc, Formula IId, Formula IIe or Formula IIf:

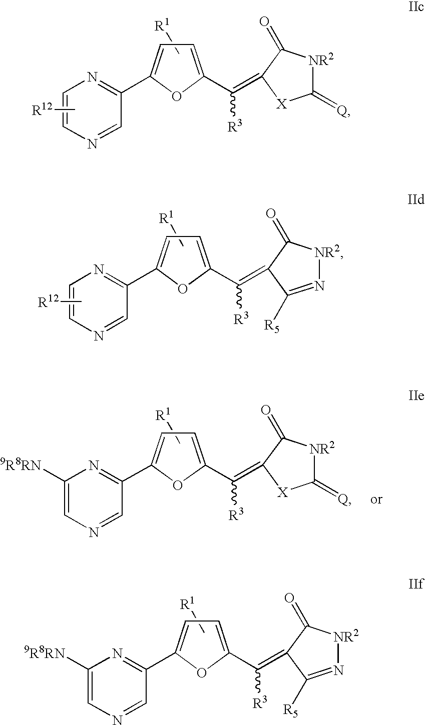

or a pharmaceutically acceptable salt thereof, wherein

R¹, R², R³, R⁵, R⁸, R⁹, and X are defined as for Formula II,

Q is O or S, and

R¹² is —CONR⁸R⁹, —CONR⁸R⁹, —SO₂NR⁸R⁹, —NR⁸R⁹, —NR⁸COR⁸, or —NR⁸CONR⁸R⁹.

In some embodiments of Formulae II-A, II-B, IIa, IIb, IIe, IId, IIe, and IIf, R¹ and R³ are H. In other embodiments of Formulae II-A, II-B, IIa, IIb, IIe, IId, IIe, and IIf, R² is H. In preferred embodiments of Formulae II-A, II-B, IIa, IIb, IIe, IId, IIe, and IIf, each of R¹, R² and R³ is H.

In further embodiments, the invention provides a compound of Formula III having the structure of Formula III-A, III-B, IIIa or Mb:

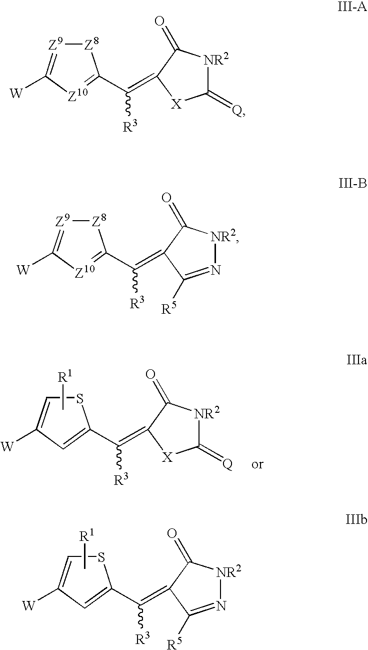

or a pharmaceutically acceptable salt thereof,

wherein Z⁸, Z⁹, Z¹⁰, R¹, R², R³, R⁵, W, and X are defined as for Formula III; and

Q is O or S.

In certain preferred embodiments, the compound of Formula III has the structure of Formula IIIc, Formula IIId, Formula IIIe or Formula IIIf:

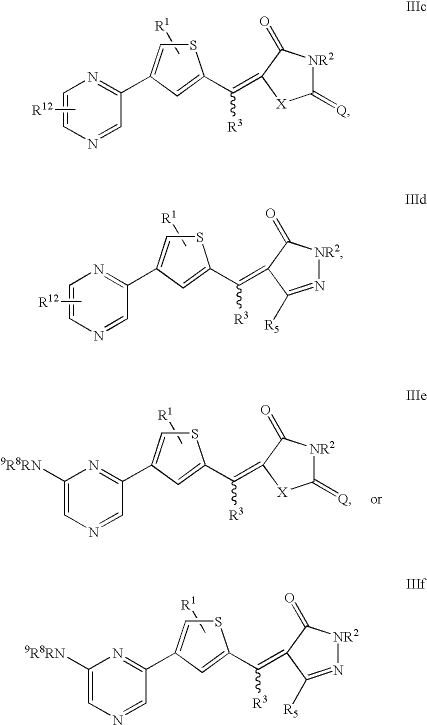

or a pharmaceutically acceptable salt thereof, wherein

R¹, R², R³, R⁵, R⁸, R⁹, and X are defined as for Formula III,

Q is O or S, and

R¹² is —CONR⁸R⁹, —CONR⁸NR⁸R⁹, —SO₂NR⁸R⁹, —NR⁸R⁹, —NR⁸COR⁸, or —NR⁸CONR⁸R⁹.

In some embodiments of Formulae III-A, III-B, IIIa, IIIb, IIIc, IIId, IIIe, and IIIf, R¹ and R³ are H. In other embodiments of Formulae III-A, III-B, IIIa, IIIb, IIIc, IIId, IIIe, and IIIf, R² is H. In preferred embodiments of Formulae III-A, III-B, IIIa, IIIb, IIIc, IIId, IIIe, and IIIf, each of R¹, R² and R³ is H.

In other embodiments of Formula I, Formula II and Formula III, W can be a group of the formula —NR¹⁰R¹¹, where R¹⁰ and R¹¹ are as described above. Typically, R¹⁰ and R¹¹ are not both H. In certain of these embodiments, R¹⁰ is H, Me, or an acyl group such as formyl, acetyl, methoxyacetyl, benzoyl, or trifluoroacetyl. Such acylated compounds may be active as kinase inhibitors, or they can serve as prodrugs for compounds wherein R¹⁰ is H. In these embodiments, R¹¹ can be an optionally substituted alkyl group, or an aryl or heteroaryl group, such as phenyl, pyridinyl, pyrimidinyl, pyrazinyl, and the like, which can be optionally substituted. Suitable optionally substituted alkyl groups include C1-C6 alkyls, e.g., methyl, ethyl, butyl, propyl, isopropyl, t-butyl, fluoroethyl, methoxyethyl, isobutyl, and the like. In certain embodiments, the aryl or heteroaryl group is substituted by at least one non-H substituent group. R¹¹ can also be such an aryl or heteroaryl group that is connected to Ne through a C1-C4 alkylene chain; e.g., it can be imidazolylmethyl, phenylethyl, and the like. In specific embodiments, the aryl is phenyl, and is substituted by at least one non-H substituent, often at the position that is meta or para to the point where the phenyl is connected to the N of NR¹⁰R¹¹.

The compounds of the invention often have ionizable groups so as to be capable of preparation as salts. In that case, wherever reference is made to the compound, it is understood in the art that a pharmaceutically acceptable salt may also be used. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art. In some cases, the compounds may contain both an acidic and a basic functional group, in which case they may have two ionized groups and yet have no net charge.

In another aspect, the invention provides a pharmaceutical composition comprising any of the above-described compound, admixed with a pharmaceutically acceptable excipient.

In another aspect, the invention provides a method to treat cancer, a vascular disorder, inflammation, a pathogenic infection, or an immunological disorder, comprising administering to a subject in need of such treatment, an effective amount of any of the above-described compounds.

In another aspect, the invention provides the use of any of the compounds described herein, or a pharmaceutical composition or medicament comprising such a compound, in a method to treat cancer, a vascular disorder, inflammation, a pathogenic infection, or an immunological disorder comprising administering such a compound to a subject in need of such treatment.

In another aspect, the invention provides a compound according to any of the formulae provided herein for use in therapy. In some embodiments, the compound is for use in the treatment of a vascular disorder, inflammation, a pathogenic infection, or an immunological disorder. In other embodiments, the compound is for use in the treatment of cancer.

The compounds of the invention are useful as medicaments, and are useful for the manufacture of medicaments, including medicaments to treat conditions disclosed herein, such as cancers, inflammatory conditions, infections, pain, and immunological disorders.

The terms “treat” and “treating” as used herein refer to ameliorating, alleviating, lessening, and removing symptoms of a disease or condition. A candidate molecule or compound described herein may be in a therapeutically effective amount in a formulation or medicament, which is an amount that can lead to a biological effect, such as apoptosis of certain cells (e.g., cancer cells), reduction of proliferation of certain cells, or lead to ameliorating, alleviating, lessening, or removing symptoms of a disease or condition, for example. The terms also can refer to reducing or stopping a cell proliferation rate (e.g., slowing or halting tumor growth) or reducing the number of proliferating cancer cells (e.g., removing part or all of a tumor).

These terms also are applicable to reducing a titre of a microorganism in a system (i.e., cell, tissue, or subject) infected with a microorganism, reducing the rate of microbial propagation, reducing the number of symptoms or an effect of a symptom associated with the microbial infection, and/or removing detectable amounts of the microbe from the system. Examples of microorganisms include but are not limited to virus, bacterium and fungus.

Compounds of the formulae provided herein are active as inhibitors of CK2, and are thus useful to treat infections by certain pathogens, including protozoans and viruses. The invention thus provides methods for treating protozoal disorders such as protozoan parasitosis, including infection by parasitic protozoa responsible for neurological disorders such as schizophrenia, paranoia, and encephalitis in immunocompromised patients, as well as Chagas' disease. It also provides methods to treat various viral diseases, including human immunodeficiency virus type 1 (HIV-1), human papilloma viruses (HPVs), herpes simplex virus (HSV), Epstein-Barr virus (EBV), human cytomegalovirus, hepatitis C and B viruses, influenza virus, Borna disease virus, adenovirus, coxsackievirus, coronavirus and varicella zoster virus. The methods for treating these disorders comprise administering to a subject in need thereof an effective amount of a compound of Formula I, II or III.

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

The invention in part provides pharmaceutical compositions comprising at least one compound within the scope of the invention as described herein, and methods of using compounds described herein.

In addition, the invention in part provides methods for identifying a candidate molecule that interacts with a CK2 and/or Pim, which comprises contacting a composition containing a CK2 or Pim protein and a molecule described herein with a candidate molecule and determining whether the amount of the molecule described herein that interacts with the protein is modulated, whereby a candidate molecule that modulates the amount of the molecule described herein that interacts with the protein is identified as a candidate molecule that interacts with the protein.

Also provided by the invention are methods for modulating certain protein kinase activities. Protein kinases catalyze the transfer of a gamma phosphate from adenosine triphosphate to a serine or threonine amino acid (serine/threonine protein kinase), tyrosine amino acid (tyrosine protein kinase), tyrosine, serine or threonine (dual specificity protein kinase) or histidine amino acid (histidine protein kinase) in a peptide or protein substrate. Thus, included herein are methods which comprise contacting a system comprising a protein kinase protein with a compound described herein in an amount effective for modulating (e.g., inhibiting) the activity of the protein kinase. In some embodiments, the activity of the protein kinase is the catalytic activity of the protein (e.g., catalyzing the transfer of a gamma phosphate from adenosine triphosphate to a peptide or protein substrate). In certain embodiments, provided are methods for identifying a candidate molecule that interacts with a protein kinase, which comprise: contacting a composition containing a protein kinase and a compound described herein with a candidate molecule under conditions in which the compound and the protein kinase interact, and determining whether the amount of the compound that interacts with the protein kinase is modulated relative to a control interaction between the compound and the protein kinase without the candidate molecule, whereby a candidate molecule that modulates the amount of the compound interacting with the protein kinase relative to the control interaction is identified as a candidate molecule that interacts with the protein kinase. Systems in such embodiments can be a cell-free system or a system comprising cells (e.g., in vitro). The protein kinase, the compound or the molecule in some embodiments is in association with a solid phase. In certain embodiments, the interaction between the compound and the protein kinase is detected via a detectable label, where in some embodiments the protein kinase comprises a detectable label and in certain embodiments the compound comprises a detectable label. The interaction between the compound and the protein kinase sometimes is detected without a detectable label.

Provided also are compositions of matter comprising a protein kinase and a compound described herein. In some embodiments, the protein kinase in the composition is a serine-threonine protein kinase. In some embodiments, the protein kinase in the composition is, or contains a subunit (e.g., catalytic subunit, SH2 domain, SH3 domain) of, CK2 or a Pim subfamily protein kinase (e.g., PIM1, PIM2, PIM3). In certain embodiments the composition is cell free and sometimes the protein kinase is a recombinant protein.

The protein kinase can be from any source, such as cells from a mammal, ape or human, for example. Examples of serine-threonine protein kinases that can be inhibited, or may potentially be inhibited, by compounds disclosed herein include without limitation human versions of CK2, CK2α2, and Pim subfamily kinases (e.g., PIM1, PIM2, PIM3). A serine-threonine protein kinase sometimes is a member of a sub-family containing one or more of the following amino acids at positions corresponding to those listed in human CK2: leucine at position 45, methionine at position 163 and isoleucine at position 174. Examples of such protein kinases include without limitation human versions of CK2, STK10, HIPK2, HIPK3, DAPK3, DYK2 and PIM-1. Nucleotide and amino acid sequences for protein kinases and reagents are publicly available (e.g., World Wide Web URLs ncbi.nlm.nih.gov/sites/entrez/and Invitrogen.com). For example, various nucleotide sequences can be accessed using the following accession numbers: NM_{—}002648.2 and NP_{—}002639.1 for PIM1; NM_{—}006875.2 and NP_{—}006866.2 for PIM2; XM_{—}938171.2 and XP_{—}943264.2 for PIM3.

The invention also in part provides methods for treating a condition related to aberrant cell proliferation. For example, provided are methods of treating a cell proliferative condition in a subject, which comprises administering a compound described herein to a subject in need thereof in an amount effective to treat the cell proliferative condition. The subject may be a research animal (e.g., rodent, dog, cat, monkey), optionally containing a tumor such as a xenograft tumor (e.g., human tumor), for example, or may be a human. A cell proliferative condition sometimes is a tumor or non-tumor cancer, including but not limited to, cancers of the colorectum, breast, lung, liver, pancreas, lymph node, colon, prostate, brain, head and neck, skin, liver, kidney, blood and heart (e.g., leukemia, lymphoma, carcinoma).

Compounds and compositions of the invention may be used alone or in combination with anticancer or other agents, such as a palliative agents, that are typically administered to a patient being treated for cancer, as further described herein.

Also provided are methods for treating a condition related to inflammation or pain. For example, methods are provided for treating pain in a subject, which comprise administering a compound described herein to a subject in need thereof in an amount effective to treat the pain. Provided also are methods of treating inflammation in a subject, which comprise administering a compound described herein to a subject in need thereof in an amount effective to treat the inflammation. The subject may be a research animal (e.g., rodent, dog, cat, monkey), for example, or may be a human. Conditions associated with inflammation and pain include without limitation acid reflux, heartburn, acne, allergies and allergen sensitivities, Alzheimer's disease, asthma, atherosclerosis, bronchitis, carditis, celiac disease, chronic pain, Crohn's disease, cirrhosis, colitis, dementia, dermatitis, diabetes, dry eyes, edema, emphysema, eczema, fibromyalgia, gastroenteritis, gingivitis, heart disease, hepatitis, high blood pressure, insulin resistance, interstitial cystitis, joint pain/arthritis/rheumatoid arthritis, metabolic syndrome (syndrome X), myositis, nephritis, obesity, osteopenia, glomerulonephritis (GN), juvenile cystic kidney disease, and type I nephronophthisis (NPHP), osteoporosis, Parkinson's disease, Guam-Parkinson dementia, supranuclear palsy, Kuf's disease, and Pick's disease, as well as memory impairment, brain ischemic, and schizophrenia, periodontal disease, polyarteritis, polychondritis, psoriasis, scleroderma, sinusitis, Sjögren's syndrome, spastic colon, systemic candidiasis, tendonitis, urinary track infections, vaginitis, inflammatory cancer (e.g., inflammatory breast cancer) and the like.

Methods for determining and monitoring effects of compounds herein on pain or inflammation are known. For example, formalin-stimulated pain behaviors in research animals can be monitored after administration of a compound described herein to assess treatment of pain (e.g., Li et al., Pain 115(1-2): 182-90 (2005)). Also, modulation of pro-inflammatory molecules (e.g., IL-8, GRO-alpha, MCP-1, TNFalpha and iNOS) can be monitored after administration of a compound described herein to assess treatment of inflammation (e.g., Parhar et al., Int J Colorectal Dis. 22(6): 601-9 (2006)), for example. Thus, also provided are methods for determining whether a compound herein reduces inflammation or pain, which comprise contacting a system with a compound described herein in an amount effective for modulating (e.g., inhibiting) the activity of a pain signal or inflammation signal.

Provided also are methods for identifying a compound that reduces inflammation or pain, which comprise: contacting a system with a compound of Formula I; and detecting a pain signal or inflammation signal, whereby a compound that modulates the pain signal relative to a control molecule is identified as a compound that reduces inflammation of pain. Non-limiting examples of pain signals are formalin-stimulated pain behaviors and examples of inflammation signals include without limitation a level of a pro-inflammatory molecule. The invention thus in part pertains to methods for modulating angiogenesis in a subject, and methods for treating a condition associated with aberrant angiogenesis in a subject. proliferative diabetic retinopathy.

CK2 has also been shown to play a role in the pathogenesis of atherosclerosis, and may prevent atherogenesis by maintaining laminar shear stress flow. CK2 plays a role in vascularization, and has been shown to mediate the hypoxia-induced activation of histone deacetylases (HDACs). CK2 is also involved in diseases relating to skeletal muscle and bone tissue, including, e.g., cardiomyocyte hypertrophy, heart failure, impaired insulin signaling and insulin resistance, hypophosphatemia and inadequate bone matrix mineralization.

Thus in one aspect, the invention provides methods to treat each of these conditions, comprising administering to a subject in need of such treatment an effect amount of a CK2 inhibitor, such as a compound of Formula I or Formula II as described herein.

The invention also in part pertains to methods for modulating an immune response in a subject, and methods for treating a condition associated with an aberrant immune response in a subject. Thus, provided are methods for determining whether a compound herein modulates an immune response, which comprise contacting a system with a compound described herein in an amount effective for modulating (e.g., inhibiting) an immune response or a signal associated with an immune response. Signals associated with immunomodulatory activity include, e.g., stimulation of T-cell proliferation, suppression or induction of cytokines, including, e.g., interleukins, interferon-γ and TNF. Methods of assessing immunomodulatory activity are known in the art.

Also provided are methods for treating a condition associated with an aberrant immune response in a subject, which comprise administering a compound described herein to a subject in need thereof in an amount effective to treat the condition. Conditions characterized by an aberrant immune response include without limitation, organ transplant rejection, asthma, autoimmune disorders, including rheumatoid arthritis, multiple sclerosis, myasthenia gravis, systemic lupus erythematosus, scleroderma, polymyositis, mixed connective tissue disease (MCTD), Crohn's disease, and ulcerative colitis. In certain embodiments, an immune response may be modulated by administering a compound herein in combination with a molecule that modulates (e.g., inhibits) the biological activity of an mTOR pathway member or member of a related pathway (e.g., mTOR, PI3 kinase, AKT). In certain embodiments the molecule that modulates the biological activity of an mTOR pathway member or member of a related pathway is rapamycin. In certain embodiments, provided herein is a composition comprising a compound described herein in combination with a molecule that modulates the biological activity of an mTOR pathway member or member of a related pathway, such as rapamycin, for example.

In certain embodiments of the present invention, the compound is a compound of Formula Ia, and in certain embodiments it is a compound of Formula Ib. In other embodiments, the compound is a compound of one of Formulae Ic, Id, Ie or If.

In other embodiments of the present invention, the compound is a compound of Formula IIa, and in certain embodiments it is a compound of Formula IIb. In other embodiments, the compound is a compound of one of Formulae IIc, IId, IIe or

In further embodiments of the present invention, the compound is a compound of Formula IIIa, and in certain embodiments it is a compound of Formula Mb. In other embodiments, the compound is a compound of one of Formulae IIIc, IIId, IIIe or IIIf.

Any suitable formulation of a compound described above can be prepared for administration by methods known in the art. Selection of useful excipients or carriers can be achieved without undue experimentation, based on the desired route of administration and the physical properties of the compound to be administered.

Any suitable route of administration may be used, as determined by a treating physician, including, but not limited to, oral, parenteral, intravenous, intramuscular, transdermal, topical and subcutaneous routes. Depending on the subject to be treated, the mode of administration, and the type of treatment desired—e.g., prevention, prophylaxis, therapy; the compounds are formulated in ways consonant with these parameters. Preparation of suitable formulations for each route of administration are known in the art. A summary of such formulation methods and techniques is found in Remington's Pharmaceutical Sciences, latest edition, Mack Publishing Co., Easton, Pa. The formulation of each substance or of the combination of two substances will frequently include a diluent as well as, in some cases, adjuvants, buffers, preservatives and the like. The substances to be administered can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised, and can be applied to compounds of the invention. See, for example, U.S. Pat. No. 5,624,677, the methods of which are incorporated herein by reference.

Systemic administration may also include relatively noninvasive methods such as the use of suppositories, transdermal patches, transmucosal delivery and intranasal administration. Oral administration is also suitable for compounds of the invention. Suitable forms include syrups, capsules, tablets, as is understood in the art.

For administration to animal or human subjects, the appropriate dosage of a compound described above often is 0.01-15 mg/kg, and sometimes 0.1-10 mg/kg. In some embodiments, a suitable dosage of the compound of the invention for an adult patient will be between 1 and 500 mg per dose, frequently between 10 and 300 mg, and the dosage may be administered 1-4 times per day. Dosage levels are dependent on the nature of the condition, drug efficacy, the condition of the patient, the judgment of the practitioner, and the frequency and mode of administration; however, optimization of such parameters is within the ordinary level of skill in the art.

### Therapeutic Combinations

Compounds of the invention may be used alone or in combination with another therapeutic agent. The invention provides methods to treat conditions such as cancer, inflammation and immune disorders by administering to a subject in need of such treatment a therapeutically effective amount of a therapeutic agent useful for treating said disorder and administering to the same subject a therapeutically effective amount of a modulator of the present invention. A CK2 and/or Pim modulator is an agent that inhibits or enhances a biological activity of a CK2 protein, a Pim protein or both, and is generically referred to hereafter as a “modulator.” Compounds of Formula I, Formula II and Formula III are exemplary ‘modulators.’ The therapeutic agent and the modulator may be administered together, either as separate pharmaceutical compositions or admixed in a single pharmaceutical composition. The therapeutic agent and the modulator may also be administered separately, including at different times and with different frequencies. The modulator may be administered by any known route, such as orally, intravenously, intramuscularly, nasally, and the like; and the therapeutic agent may also be administered by any conventional route. In many embodiments, at least one and optionally both of the modulator and the therapeutic agent may be administered orally. Preferably, the modulator is an inhibitor, and it may inhibit either one of CK2 and Pim, or both of them to provide the treatment effects described herein.

In certain embodiments, a “modulator” as described above may be used in combination with a therapeutic agent that can act by binding to regions of DNA that can form certain quadruplex structures. In such embodiments, the therapeutic agents have anticancer activity on their own, but their activity is enhanced when they are used in combination with a modulator. This synergistic effect allows the therapeutic agent to be administered in a lower dosage while achieving equivalent or higher levels of at least one desired effect.

A modulator may be separately active for treating a cancer. For combination therapies described above, when used in combination with a therapeutic agent, the dosage of a modulator will frequently be two-fold to ten-fold lower than the dosage required when the modulator is used alone to treat the same condition or subject. Determination of a suitable amount of the modulator for use in combination with a therapeutic agent is readily determined by methods known in the art.

Compounds and compositions of the invention may be used in combination with anticancer or other agents, such as palliative agents, that are typically administered to a patient being treated for cancer. Such “anticancer agents” include, e.g., classic chemotherapeutic agents, as well as molecular targeted therapeutic agents, biologic therapy agents, and radiotherapeutic agents.

When a compound or composition of the invention is used in combination with an anticancer agent to another agent, the present invention provides, for example, simultaneous, staggered, or alternating treatment. Thus, the compound of the invention may be administered at the same time as an anticancer agent, in the same pharmaceutical composition; the compound of the invention may be administered at the same time as the anticancer agent, in separate pharmaceutical compositions; the compound of the invention may be administered before the anticancer agent, or the anticancer agent may be administered before the compound of the invention, for example, with a time difference of seconds, minutes, hours, days, or weeks.

In examples of a staggered treatment, a course of therapy with the compound of the invention may be administered, followed by a course of therapy with the anticancer agent, or the reverse order of treatment may be used, and more than one series of treatments with each component may also be used. In certain examples of the present invention, one component, for example, the compound of the invention or the anticancer agent, is administered to a mammal while the other component, or its derivative products, remains in the bloodstream of the mammal. For example, a compound for formulae I, II, or III may be administered while the anticancer agent or its derivative products remains in the bloodstream, or the anticancer agent may be administered while the compound of formulae I, II, or III or its derivatives remains in the bloodstream. In other examples, the second component is administered after all, or most of the first component, or its derivatives, have left the bloodstream of the mammal.

The compound of the invention and the anticancer agent may be administered in the same dosage form, e.g., both administered as intravenous solutions, or they may be administered in different dosage forms, e.g., one compound may be administered topically and the other orally. A person of ordinary skill in the art would be able to discern which combinations of agents would be useful based on the particular characteristics of the drugs and the cancer involved.

Anticancer agents useful in combination with the compounds of the present invention may include agents selected from any of the classes known to those of ordinary skill in the art, including, but not limited to, antimicrotubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; nonreceptor tyrosine kinase angiogenesis inhibitors; immunotherapeutic agents; pro-apoptotic agents; and cell cycle signaling inhibitors; other agents.

Anti-microtubule or anti-mitotic agents are phase specific agents that are typically active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that are believed to operate at the G2/M phases of the cell cycle. It is believed that the diterpenoids stabilize the p-tubulin subunit of the microtubules, by binding with this protein. Disassembly of the protein appears then to be inhibited with mitosis being arrested and cell death following.

Examples of diterpenoids include, but are not limited to, taxanes such as paclitaxel, docetaxel, larotaxel, ortataxel, and tesetaxel. Paclitaxel is a natural diterpene product isolated from the Pacific yew tree Taxus brevifolia and is commercially available as an injectable solution TAXOL®. Docetaxel is a semisynthetic derivative of paclitaxel q. v., prepared using a natural precursor, 10-deacetyl-baccatin III, extracted from the needle of the European Yew tree. Docetaxel is commercially available as an injectable solution as TAXOTERE®.

Vinca alkaloids are phase specific anti-neoplastic agents derived from the periwinkle plant. Vinca alkaloids that are believed to act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, vindesine, and vinorelbine. Vinblastine, vincaleukoblastine sulfate, is commercially available as VELBAN® as an injectable solution. Vincristine, vincaleukoblastine 22-oxo-sulfate, is commercially available as ONCOVIN® as an injectable solution. Vinorelbine, is commercially available as an injectable solution of vinorelbine tartrate (NAVELBINE®), and is a semisynthetic vinca alkaloid derivative.

Platinum coordination complexes are non-phase specific anti-cancer agents, which are interactive with DNA. The platinum complexes are believed to enter tumor cells, undergo, aquation and form intra- and interstrand crosslinks with DNA causing adverse biological effects to the tumor. Platinum-based coordination complexes include, but are not limited to cisplatin, carboplatin, nedaplatin, oxaliplatin, satraplatin, and (SP-4-3)-(cis)-amminedichloro[2-methylpyridine] platinum(II). Cisplatin, cis-diamminedichloroplatinum, is commercially available as PLATINOL® as an injectable solution. Carboplatin, platinum, diammine [1,1-cyclobutane-dicarboxylate(2-)-0,0′], is commercially available as PARAPLATIN® as an injectable solution.

Alkylating agents are generally non-phase specific agents and typically are strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, alkyl sulfonates such as busulfan; ethyleneimine and methylmelamine derivatives such as altretamine and thiotepa; nitrogen mustards such as chlorambucil, cyclophosphamide, estramustine, ifosfamide, mechlorethamine, melphalan, and uramustine; nitrosoureas such as carmustine, lomustine, and streptozocin; triazenes and imidazotetrazines such as dacarbazine, procarbazine, temozolamide, and temozolomide. Cyclophosphamide, 2-[bis(2-chloroethyl)-amino]tetrahydro-2H-1,3,2-oxazaphosphorine 2-oxide monohydrate, is commercially available as an injectable solution or tablets as CYTOXAN®. Melphalan, 4-[bis(2-chloroethyl)amino]-L-phenylalanine, is commercially available as an injectable solution or tablets as ALKERAN®. Chlorambucil, 4-[bis(2-chloroethyl)amino]-benzenebutanoic acid, is commercially available as LEUKERAN® tablets. Busulfan, 1,4-butanediol dimethanesulfonate, is commercially available as MYLERAN® TABLETS. Carmustine, 1,3-[bis(2-chloroethyl)-1-nitrosourea, is commercially available as single vials of lyophilized material as BiCNU®, 5-(3,3-dimethyl-1-triazeno)-imidazole-4-carboxamide, is commercially available as single vials of material as DTIC-Dome®.

Anti-tumor antibiotics are non-phase specific agents which are believed to bind or intercalate with DNA. This may result in stable DNA complexes or strand breakage, which disrupts ordinary function of the nucleic acids, leading to cell death. Examples of anti-tumor antibiotic agents include, but are not limited to, anthracyclines such as daunorubicin (including liposomal daunorubicin), doxorubicin (including liposomal doxorubicin), epirubicin, idarubicin, and valrubicin; streptomyces-related agents such as bleomycin, actinomycin, mithramycin, mitomycin, porfiromycin; and mitoxantrone. Dactinomycin, also know as Actinomycin D, is commercially available in injectable form as COSMEGEN®. Daunorubicin, (8S-cis-)-8-acetyl-10-[(3-amino-2,3,6-trideoxy-α-L-lyxohexopyranosyl)oxy]-7,8,9,10-tetrahydro-6,8,11-trihydroxy-1-methoxy-5,12-naphthacenedione hydrochloride, is commercially available as a liposomal injectable form as DAUNOXOME® or as an injectable as CERUBIDINE®. Doxorubicin, (8S,10S)-10-[(R3-amino-2,3,6-trideoxy-α-L-lyxohexopyranosyl)oxy]-8-glycoloyl, 7,8,9,10-tetrahydro-6,8,11-trihydroxy-1-methoxy-5,12-naphthacenedione hydrochloride, is commercially available in an injectable form as RUBEX® or ADRIAMYCIN RDF®. Bleomycin, a mixture of cytotoxic glycopeptide antibiotics isolated from a strain of Streptomyces verticil/us, is commercially available as BLENOXANE®.

Topoisomerase II inhibitors include, but are not limited to, epipodophyllotoxins, which are phase specific anti-neoplastic agents derived from the mandrake plant. Epipodophyllotoxins typically affect cells in the S and G2 phases of the cell cycle by forming a ternary complex with topoisomerase II and DNA causing DNA strand breaks. The strand breaks accumulate and cell death follows. Examples of epipodophyllotoxins include, but are not limited to, etoposide, teniposide, and amsacrine. Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Teniposide, 4′-demethyl-epipodophyllotoxin 9[4,6-0-(R)-thenylidene-β-D-glucopyranoside], is commercially available as an injectable solution as VUMON® and is commonly known as VM-26.

Antimetabolite neoplastic agents are phase specific anti-neoplastic agents that typically act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Anti-metabolites, include purine analogs, such as fludarabine, cladribine, chlorodeoxyadenosine, clofarabine, mercaptopurine, pentostatin, erythrohydroxynonyladenine, fludarabine phosphate and thioguanine; pyrimidine analogs such as fluorouracil, gemcitabine, capecitabine, cytarabine, azacitidine, edatrexate, floxuridine, and troxacitabine; antifolates, such as methotrexate, pemetrexed, raltitrexed, and trimetrexate. Cytarabine, 4-amino-1-p-D-arabinofuranosyl-2(1H)-pyrimidinone, is commercially available as CYTOSAR-U® and is commonly known as Ara-C. Mercaptopurine, 1,7-dihydro-6H-purine-6-thione monohydrate, is commercially available as PURINETHOL®. Thioguanine, 2-amino-1,7-dihydro-6H-purine-6-thione, is commercially available as TABLOID®. Gemcitabine, 2′-deoxy-2′,2′-difluorocytidine monohydrochloride (p-isomer), is commercially available as GEMZAR®.

Topoisomerase I inhibitors including, camptothecin and camptothecin derivatives. Examples of topoisomerase I inhibitors include, but are not limited to camptothecin, topotecan, irinotecan, rubitecan, belotecan and the various optical forms (i.e., (R), (S) or (R,S)) of 7-(4-methylpiperazino-methylene)-10,11-ethylenedioxy-camptothecin, as described in U.S. Pat. Nos. 6,063,923; 5,342,947; 5,559,235; 5,491,237 and pending U.S. patent application Ser. No. 08/977,217 filed Nov. 24, 1997. Irinotecan HCl, (4S)-4,11-diethyl-4-hydroxy-9-[(4-piperidinopiperidino)-carbonyloxy]-1H-pyrano[3′,4′,6,7]indolizino[1,2-b]quinoline-3,14(4H,12H)-dione hydrochloride, is commercially available as the injectable solution CAMPTOSAR®. Irinotecan is a derivative of camptothecin which binds, along with its active metabolite 8N-38, to the topoisomerase I-DNA complex. Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, androgens such as fluoxymesterone and testolactone; antiandrogens such as bicalutamide, cyproterone, flutamide, and nilutamide; aromatase inhibitors such as aminoglutethimide, anastrozole, exemestane, formestane, vorazole, and letrozole; corticosteroids such as dexamethasone, prednisone and prednisolone; estrogens such as diethylstilbestrol; antiestrogens such as fulvestrant, raloxifene, tamoxifen, toremifene, droloxifene, and iodoxyfene, as well as selective estrogen receptor modulators (SERMS) such those described in U.S. Pat. Nos. 5,681,835, 5,877,219, and 6,207,716; 5□-reductases such as finasteride and dutasteride; gonadotropin-releasing hormone (GnRH) and analogues thereof which stimulate the release of leutinizing hormone (LH) and/or follicle stimulating hormone (FSH), for example LHRH agonists and antagonists such as buserelin, goserelin, leuprolide, and triptorelin; progestins such as medroxyprogesterone acetate and megestrol acetate; and thyroid hormones such as levothyroxine and liothyronine.

Signal transduction pathway inhibitors are those inhibitors, which block or inhibit a chemical process which evokes an intracellular change, such as cell proliferation or differentiation. Signal tranduction inhibitors useful in the present invention include, e.g., inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases. Receptor tyrosine kinases are transmembrane proteins having an extracellular ligand binding domain, a transmembrane domain, and a tyrosine kinase domain Receptor tyrosine kinases are involved in the regulation of cell growth and are sometimes termed growth factor receptors.

Inappropriate or uncontrolled activation of many of these kinases, for example by over-expression or mutation, has been shown to result in uncontrolled cell growth. Accordingly, the aberrant activity of such kinases has been linked to malignant tissue growth. Consequently, inhibitors of such kinases could provide cancer treatment methods.

Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene.

Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al., Drug Discov. Today (1997), 2(2):50-63; and Lofts, F. J. et al., “Growth factor receptors as targets”, New Molecular Targets for Cancer Chemotherapy, ed. Workman, Paul and Kerr, David, CRC press 1994, London. Specific examples of receptor tyrosine kinase inhibitors include, but are not limited to, sunitinib, erlotinib, gefitinib, and imatinib.

Tyrosine kinases which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S, and Corey, S. J., J. Hematotherapy & Stem Cell Res. (1999) 8(5): 465-80; and Bolen, J. B., Brugge, J. S., Annual Review of Immunology. (1997) 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E., J. Pharmacol. Toxicol. Methods. (1995), 34(3): 125-32 Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., J. Biochemistry. (1999) 126 (5): 799-803; Brodt, P, Samani, A, & Navab, R, Biochem. Pharmacol. (2000) 60:1101-1107; Massague, J., Weis-Garcia, F., Cancer Surv. (1996) 27:41-64; Philip, P. A, and Harris, A L., Cancer Treat. Res. (1995) 78: 3-27; Lackey, K. et al. Bioorg. Med. Chem. Letters, (2000) 10(3): 223-226; U.S. Pat. No. 6,268,391; and Martinez-Lacaci, I., et al., Int. J. Cancer (2000), 88(1): 44-52 Inhibitors of Phosphotidyl inositol-3 Kinase family members including blockers of PI3-kinase, ATM, DNA-PK, and Ku are also useful in the present invention. Such kinases are discussed in Abraham, R T. Current Opin. Immunol. (1996), 8(3): 412-8; Canman, C. E., Lim, D. S., Oncogene (1998) 17(25): 3301-8; Jackson, S. P., Int. J. Biochem. Cell Biol. (1997) 29(7):935-8; and Zhong, H. et al., Cancer Res. (2000) 60(6):1541-5. Also useful in the present invention are Myo-inositol signaling inhibitors such as phospholipase C blockers and Myoinositol analogues. Such signal inhibitors are described in Powis, G., and Kozikowski A, (1994) New Molecular Targets for Cancer Chemotherapy, ed., Paul Workman and David Kerr, CRC Press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R, Gervasoni, S I, Matar, P., J. Biomed. Sci. (2000) 7(4): 292-8; Ashby, M. N., Curr. Opin. Lipidol. (1998) 9(2): 99-102; and Oliff, A., Biochim. Biophys. Acta, (1999) 1423(3):C19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al., Cancer Treat. Rev., (2000) 26(4): 269-286); Herceptin® erbB2 antibody (see Stern, D F, Breast Cancer Res. (2000) 2(3):176-183); and 2CB VEGFR2 specific antibody (see Brekken, R. A. et al., Cancer Res. (2000) 60(18):5117-24).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphav beta3) that will inhibit angiogenesis; endostatin and angiostatin (non-RTK) may also prove useful in combination with the disclosed erb family inhibitors. (See Bruns, C J et al., Cancer Res. (2000), 60(11): 2926-2935; Schreiber A B, Winkler M E, & Derynck R., Science (1986) 232(4755):1250-53; Yen L. et al., Oncogene (2000) 19(31): 3460-9).

Agents used in immunotherapeutic regimens may also be useful in combination with the compounds of formulae disclosed herein. There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T, et al., Cancer Res. (2000) 60(13):3569-76; and Chen Y, et al., Cancer Res. (1998) 58(9):1965-71.

Agents used in pro-apoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family. Therefore, strategies designed to downregulate the expression of bcl-2 in tumors have demonstrated clinical benefit and are now in Phase II/III trials, namely Genta's G3139 bcl-2 antisense oligonucleotide. Such pro-apoptotic strategies using the antisense oligonucleotide strategy for bcl-2 are discussed in Waters J S, et al., J. Clin. Oncol. (2000) 18(9): 1812-23; and Kitada S, et al. Antisense Res. Dev. (1994) 4(2): 71-9.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania G R & Chang Y-T., Exp. Opin. Ther. Patents (2000) 10(2):215-30.

Other molecular targeted agents include FKBP binding agents, such as the immunosuppressive macrolide antibiotic, rapamycin; gene therapy agents, antisense therapy agents, and gene expression modulators such as the retinoids and rexinoids, e.g. adapalene, bexarotene, trans-retinoic acid, 9-cisretinoic acid, and N-(4 hydroxyphenyl)retinamide; phenotype-directed therapy agents, including: monoclonal antibodies such as alemtuzumab, bevacizumab, cetuximab, ibritumomab tiuxetan, rituximab, and trastuzumab; immunotoxins such as gemtuzumab ozogamicin, radioimmunoconjugates such as 131-tositumomab; and cancer vaccines.

Miscellaneous agents include altretamine, arsenic trioxide, gallium nitrate, hydroxyurea, levamisole, mitotane, octreotide, procarbazine, suramin, thalidomide, photodynamic compounds such as methoxsalen and sodium porfimer, and proteasome inhibitors such as bortezomib.

Biologic therapy agents include: interferons such as interferon-u2a and interferon-u2b, and interleukins such as aldesleukin, denileukin diftitox, and oprelvekin.

In addition to these anticancer agents intended to act against cancer cells, combination therapies including the use of protective or adjunctive agents, including: cytoprotective agents such as armifostine, dexrazonxane, and mesna, phosphonates such as pamidronate and zoledronic acid, and stimulating factors such as epoetin, darbeopetin, filgrastim, PEG-filgrastim, and sargramostim, are also envisioned.

The following examples illustrate and do not limit the invention.

### Example 1

### Synthesis of Compound 3: General Procedure

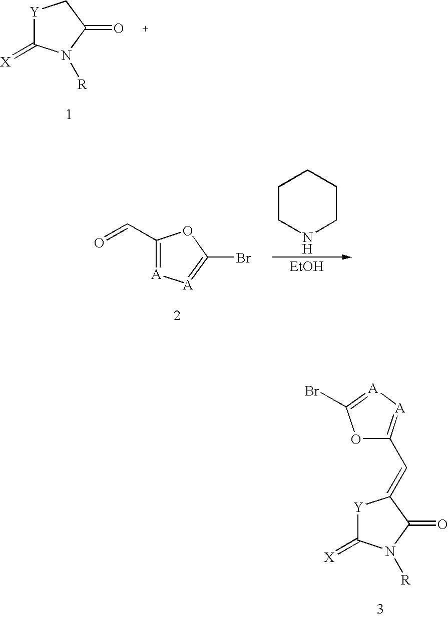

Known and readily available compounds of formula 1 react with heteroaryl aldehydes such as 2 to provide intermediates of formula 3; this reaction can be promoted by an amine such as piperidine in an alcoholic solvent. As a general example, a solution of compound 1 (1.54 mmol), aldehyde 2 (1.24 mmol) and piperidine (1.52 mmol) in EtOH (4.0 mL) is stirred at rt or to reflux. The resulting precipitate is collected by filtration to yield desired compound 3.

### Example 2

### Synthesis of Compound 4: General Arylation Procedure

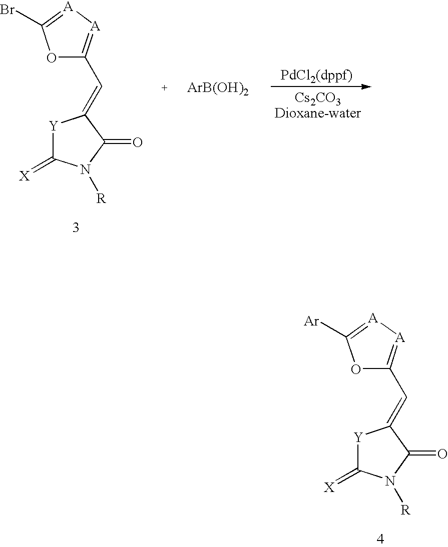

A solution of compound 3 (0.41 mmol), arylboronic acid (0.64 mmol), Cs₂CO₃ (270 mg, 0.83 mmol) and PdCl₂(dppf) (16 mg, 0.02 mmol) in H₂O/dioxane (5%, 5 mL) is heated at reflux for 6 h. The reaction mixture is diluted with H₂O (150 mL) and extracted with EtOAc (3×100 mL). The organic layer is washed with brine (100 mL) and dried over Na₂SO₄ and concentrated to yield the desired compound 4.

### Example 3

### Synthesis of Compound 4: General Procedure—Arylation Followed by Condensation

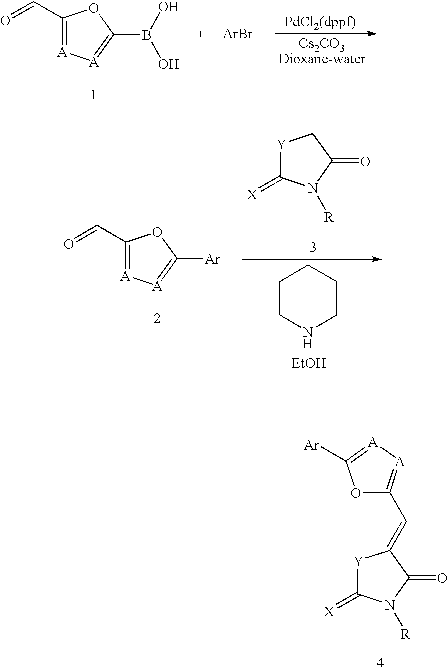

A solution of compound 1 (0.64 mmol), arylbromide (0.41 mmol), Cs₂CO₃ (270 mg, 0.83 mmol) and PdCl₂(dppf) (16 mg, 0.02 mmol) in H₂O/dioxane (5%, 5 mL) is heated at reflux for 6 h. The reaction mixture is diluted with H₂O (150 mL) and extracted with EtOAc (3×100 mL). The organic layer is washed with brine (100 mL) and dried over Na₂SO₄ and concentrated to yield the desired compound 2. A solution of compound 3 (0.12 mmol), aldehyde 2 (0.12 mmol) and piperidine (0.12 mmol) in EtOH (2.0 mL) is stirred at reflux for 30 min. The resulting precipitate is collected by filtration to yield desired compound 4.

### Example 4

### Synthesis of Compound 3: General Procedure for Making Intermediates

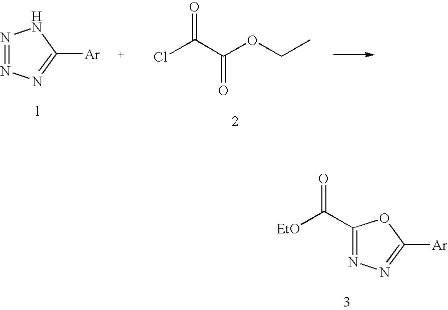

5-aryltetrazole 1 (45 mmol) and ethyl oxalyl chloride 2 (45 mmol) in dry toluene (150 mL) are refluxed for 90 min. The solvent is evaporated in vacuo and the residue is purified by flash column chromatography (silica gel, hexane/ethyl acetate 4:1 v/v) to give compound 3.

### Example 5

### Synthesis of Aryl Tetrazole 2: General Procedure for Making Aryl Tetrazoles

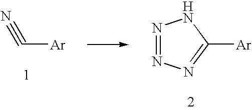

A solution of compound 1 (0.13 mmol) and azidotrimethylsilane (0.1 mL, 0.76 mmol), and ammonium chloride (21 mg, 0.39 mmol) in DMF (2 mL) is heated to 90° C. for 3 days. The mixture is cooled to room temperature, diluted with dichloromethane, washed with 1N HCl, with water, dried with Na₂SO₄, filtered and concentrated. The residue is purified on silica gel (eluted with dichloromethane) to give the desired compound 2.

### Example 6

### Synthesis of Aldehyde from Ester: General Procedure

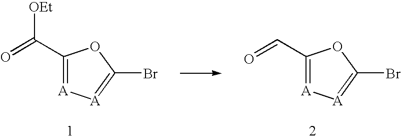

Compound 1 (2.4 mmol) is dissolved in 10 mL of anhydrous THF and then 1.2 mL of 1M solution of LiAlH4 was added to the resulting solution at 0° C. After the reaction is continued at room temperature for 30 min, 8 mL of 1N HCl is added to the reaction system, the mixture is concentrated under reduced pressure to a volume about 8 mL, the resulting concentrate is extracted with ethyl acetate and then the extract is concentrated under reduced pressure to thus give the corresponding alcohol. This alcohol is dissolved in 20 mL of methylene chloride, 1 g Molecular Sieves 4 Å and pyridinium chlorochromate (0.63 mmol) is added to the solution at 0° C., the mixture is stirred at 0° C. for 3 hours, the mixture is passed through a plug of CELITE™, followed by elution with 100 mL of diethyl ether and concentrated under reduced pressure to give compound 2.

### Example 7

### Synthesis of Compound 3: General Procedure

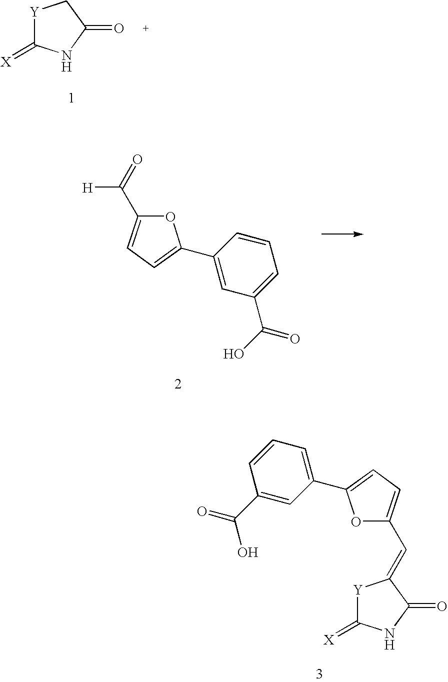

A solution of compound 1 (1.54 mmol), methyl 3-(5-formylfuran-3-yl)benzoate 2 (350 mg, 1.24 mmol) and piperidine (0.15 mL, 1.52 mmol) in EtOH (4.0 mL) is stirred at 70° C. for 30 min. The resulting precipitate is collected by filtration to yield desired compound 3.

### Example 8

### Synthesis of Compound 3: General Procedure

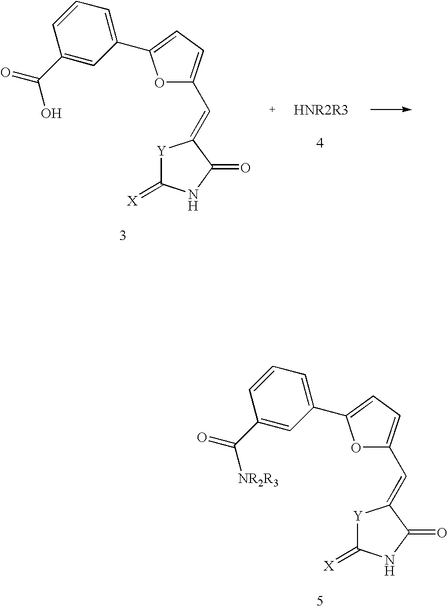

To a solution of compound 3 (0.06 mmol) and HOBt (16 mg, 0.14 mmol) in NMP (0.5 mL) is added EDCI (22 mg, 0.12 mmol). The reaction is stirred at rt for 10 min and then amine 4 (0.24 mmol) is added, followed by DIEA (0.05 mL). The reaction mixture is stirred at rt for 1 h and diluted with H₂O (10 mL). The mixture is extracted with EtOAc, dried over Na₂SO₄ and concentrated. The crude material is purified by RHPLC to give compound 5.

### Example 9

### Synthesis of 1-(5-(3-(4-methyl-1,4-diazepane-1-carbonyl)phenyl)furan-2-yl)ethanone

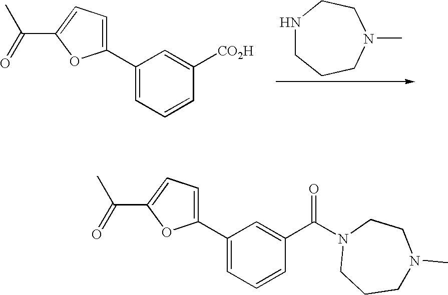

To a solution of 3-(5-acetylfuran-2-yl)benzoic acid (1.10 g, 4.78 mmol) and HOBt (1.29 g, 9.56 mmol) in DMF (10 mL) is added EDCI (1.83 g, 9.56 mmol). The reaction was stirred at rt for 10 min and then 1-methylhomopiperazine (2.38 mL, 19.12 mmol) was added followed by DIEA (3.35 mL, 619.12 mmol). The reaction mixture was stirred at rt for 1 h and diluted with H₂O (50 mL). The mixture was extracted with DCM (3×50 mL), dried over Na₂SO₄ and concentrated to give brown oil (1.70 g). LCMS (ES): m/z 442 [M+1]⁺.

### Example 10

### Synthesis of (E)-5-chloro-3-(1-(5-(3-(4-methyl-1,4-diazepane-1-carbonyl)phenyl) furan-2-yl)ethylidene)indolin-2-one

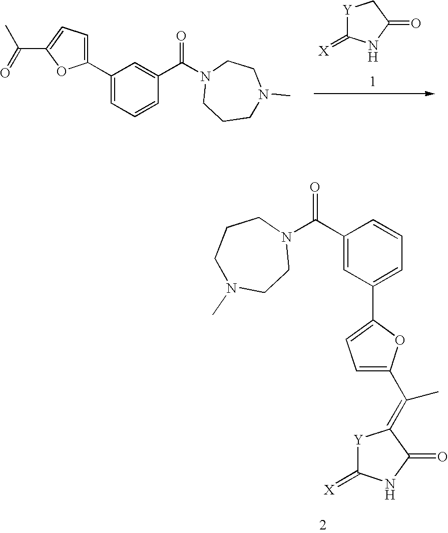

A solution of compound 1 (4 mmol), 1-(5-(3-(4-methyl-1,4-diazepane-1-carbonyl)phenyl)furan-2-yl)ethanone (1.7 g of oil) and piperidine (394 μL, 4 mmol) in toluene (15 mL) is heated at reflux with a Dean-Stark receiver for 24 hours. After evaporation of the solvent, purification by silica column chromatography (methanol gradient in dichloromethane, 0 to 5% vol) gives compound 2.

### Example 11

### Synthesis of Compound 3: General Procedure

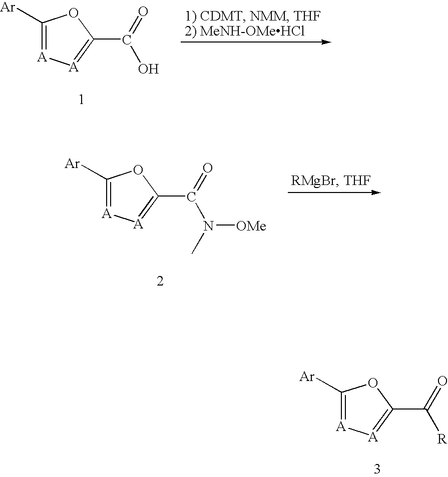

Compound 2 can be synthesized as described in Lidia De Luca et al., J. Org. Chem. 2001, 66, 2534-2537.

To a solution of the acid 1 (3.7 mmol) in THF (11 mL), at room temperature, is added 2-chloro-4,6-dimethoxy-[1,3,5]-triazine (CDMT) (4.4 mmol) and N-methylmorpholine (NMM) (11.1 mmol). A precipitate is formed during stirring, and then N,O-dimethylhydroxylamine hydrochloride (3.7 mmol) is added. The mixture is stirred for additional 8 h and then quenched with 15 mL of water and extracted two times with 7 mL of diethyl ether. The combined organic phases are washed two times with 15 mL of a saturated solution of Na₂CO₃, followed by 15 mL of a solution 1 N HCl and brine. The organic layer is dried over anhydrous Na₂SO₄ to give, after evaporation of solvent, compound 2. A solution of compound 2 (2.5 mmol) in THF (10 mL) is added at room temperature to a THF solution (11 mL) of RMgBr (2.5 mmol), stirred for additional 0.5 h, and then quenched with aqueous saturated NH₄Cl and extracted two times with 10 mL of diethyl ether. The combined organic phases are washed with 15 mL of a saturated solution of Na₂CO₃, followed by 15 mL of a solution 1 N HCl and brine. The organic layer is dried over anhydrous Na₂SO₄ to give, after evaporation of solvent, crude 3 that is further purified by flash-chromatography.

### Example 12

### Synthesis of Compound 3: General Procedure

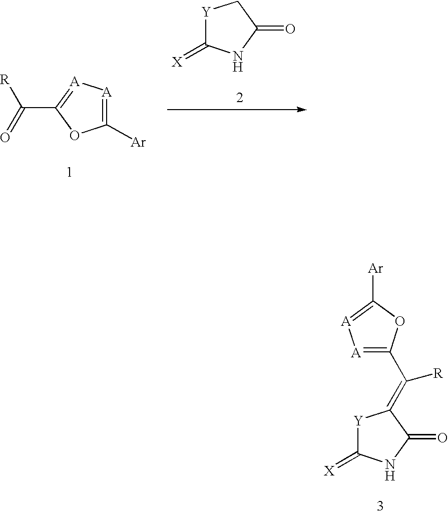

A solution of compound 2 (4 mmol) and compound 1 (4 mmol) and piperidine (394 μL, 4 mmol) in toluene (15 mL) is heated at reflux with a Dean-Stark receiver for 24 hours. After evaporation of the solvent, purification by silica column chromatography (methanol gradient in dichloromethane, 0 to 5% vol) gives compound 3.

### Example 13

### Synthesis of 4-(5-((2,5-dioxoimidazolidin-4-ylidene)methyl)furan-2-yl)benzenesulfonamide

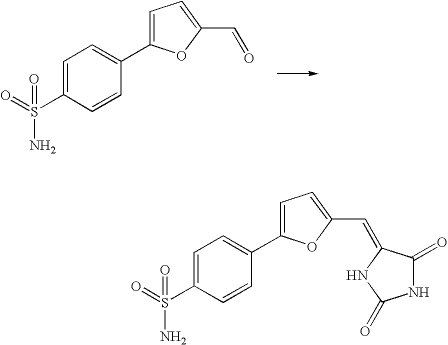

To 4-(5-formylfuran-2-yl)benzenesulfonamide (30 mg, 0.120 mmol) in EtOH was added hydantoin (12 mg, 0.120 mmol) and piperidine (12 mL, 0.120 mmol). The mixture was heated at 70° C. overnight. The solid formed was isolated by filtration and air dried to yield 4-(5-((2,5-dioxoimidazolidin-4-ylidene)methyl)furan-2-yl)benzenesulfonamide. LCMS (M+1=334).

### Example 14

### Synthesis of 4-(5-((2,4-dioxothiazolidin-5-ylidene)methyl)furan-2-yl)benzenesulfonamide

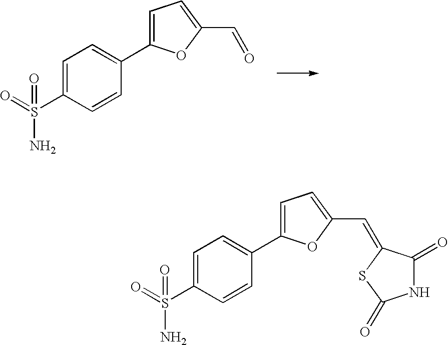

To 4-(5-formylfuran-2-yl)benzenesulfonamide (30 mg, 0.120 mmol) in EtOH was added thiazolidine-2,4-dione (14 mg, 0.120 mmol) and piperidine (12 mL, 0.120 mmol). The mixture was heated at 70° C. for several hours. The solid formed was isolated by filtration and air dried to yield 4-(5-((2,4-dioxothiazolidin-5-ylidene)methyl)furan-2-yl)benzenesulfonamide. LCMS (M+1=351).

### Example 15

### Synthesis of 4-(5-((4-oxo-2-thioxothiazolidin-5-ylidene)methyl)furan-2-yl)benzenesulfonamide

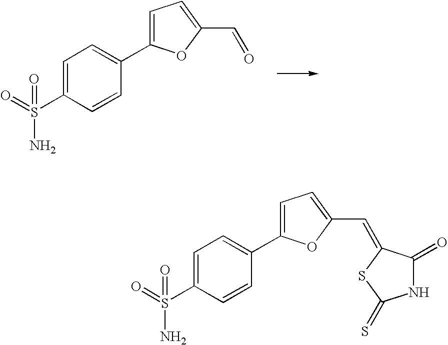

To 4-(5-formylfuran-2-yl)benzenesulfonamide (30 mg, 0.120 mmol) in EtOH was added rhodanine (16 mg, 0.120 mmol) and piperidine (12 mL, 0.120 mmol). The mixture was heated at 70° C. for several hours. The solid formed was isolated by filtration and air dried to yield 4-(5-((4-oxo-2-thioxothiazolidin-5-ylidene)methyl)furan-2-yl)benzenesulfonamide. LCMS (M+1=367).

### Example 16

### Synthesis of 4-chloro-3-(5-((4-oxo-2-thioxothiazolidin-5-ylidene)methyl)furan-2-yl)benzoic Acid

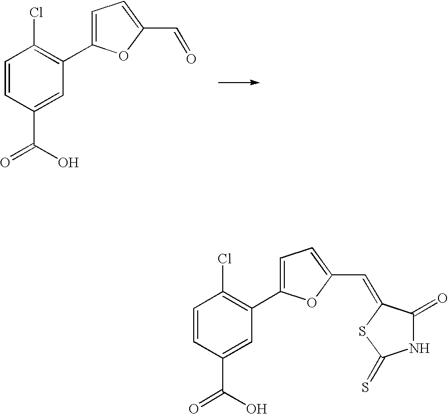

To 4-chloro-3-(5-formylfuran-2-yl)benzoic acid (80 mg, 0.32 mmol) in EtOH was added rhodanine (43 mg, 0.32 mmol) and piperidine (32 mL, 0.32 mmol). The mixture was heated at 70° C. for two hours. The solid formed was isolated by filtration to yield 4-chloro-3-(5-((4-oxo-2-thioxothiazolidin-5-ylidene)methyl)furan-2-yl)benzoic acid. LCMS (M+1=365).

### Example 17

### Synthesis of 2-chloro-5-(5-((4-oxo-2-thioxothiazolidin-5-ylidene)methyl)furan-2-yl)benzoic Acid

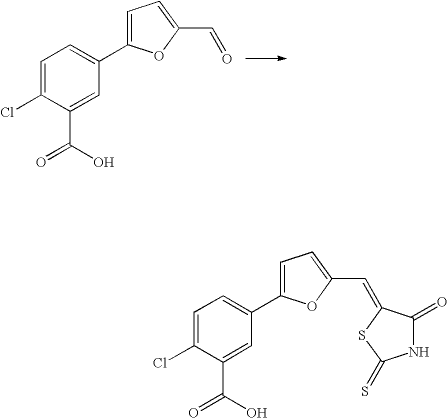

To 2-chloro-5-(5-formylfuran-2-yl)benzoic acid (80 mg, 0.32 mmol) in EtOH was added rhodanine (43 mg, 0.32 mmol) and piperidine (32 mL, 0.32 mmol). The mixture was heated at 70° C. for a couple of hours. The solid formed was isolated by filtration to yield 2-chloro-5-(5-((4-oxo-2-thioxothiazolidin-5-ylidene)methyl)furan-2-yl)benzoic acid. LCMS (M+1=365).

### Example 18

### Synthesis of 4-chloro-N-(2-(diethylamino)ethyl)-3-(5-((4-oxo-2-thioxothiazolidin-5-ylidene)methyl)furan-2-yl)benzamide

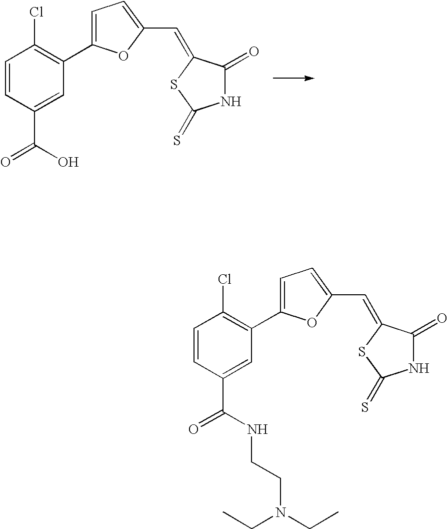

To 4-chloro-3-(5-((4-oxo-2-thioxothiazolidin-5-ylidene)methyl)furan-2-yl)benzoic acid (41 mg, 0.122 mmol) in DMF was added HBTU (64 mg, 0.168 mmol) and DIEA (59 mL, 0.337 mmol). The mixture was stirred at room temperature until no more acid was detected by LCMS. To the mixture was added N′,N′-diethylethane-1,2-diamine (19 mL, 0.135 mmol) and stirred at room temperature overnight. The solution was purified by HPLC to yield 4-chloro-N-(2-(diethylamino)ethyl)-3-(5-((4-oxo-2-thioxothiazolidin-5-ylidene)methyl)furan-2-yl)benzamide. LCMS (M+1=464).

### Example 19

### Synthesis of 4-chloro-N-(2-methoxyethyl)-3-(5-((4-oxo-2-thioxothiazolidin-5-ylidene)methyl)furan-2-yl)benzamide

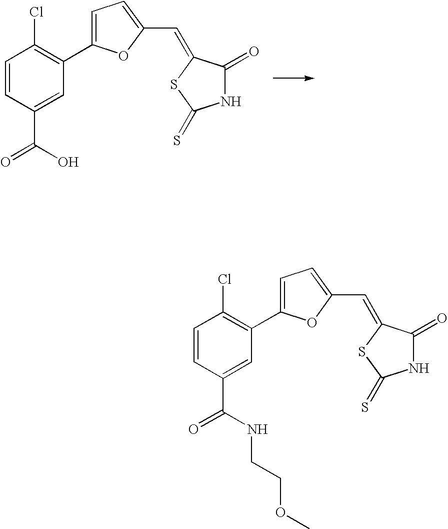

To 4-chloro-3-(5-((4-oxo-2-thioxothiazolidin-5-ylidene)methyl)furan-2-yl)benzoic acid (41 mg, 0.122 mmol) in DMF was added HBTU (64 mg, 0.168 mmol) and DIEA (59 mL, 0.337 mmol). The mixture was stirred at room temperature until no more acid was detected by LCMS. To the mixture was added 2-methoxyethanamine (12 mL, 0.135 mmol) and stirred at room temperature overnight. The solution was prepared by HPLC to yield 4-chloro-N-(2-methoxyethyl)-3-(5-((4-oxo-2-thioxothiazolidin-5-ylidene)methyl)furan-2-yl)benzamide. LCMS (M+1=423).

### Example 20

### Synthesis of 2-chloro-N-(2-(diethylamino)ethyl)-5-(5-((4-oxo-2-thioxothiazolidin-5-ylidene)methyl)furan-2-yl)benzamide

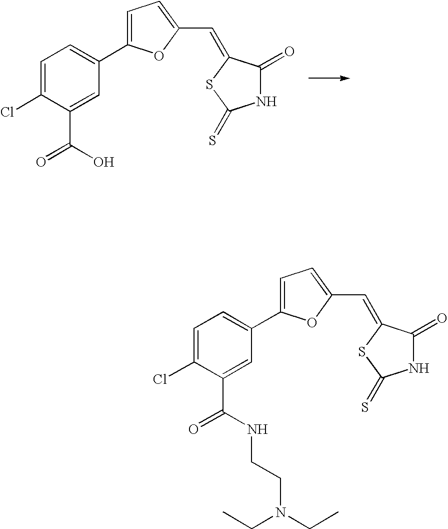

To 2-chloro-5-(5-((4-oxo-2-thioxothiazolidin-5-ylidene)methyl)furan-2-yl)benzoic acid (47 mg, 0.129 mmol) in DMF was added HBTU (73 mg, 0.193 mmol) and DIEA (67 mL, 0.386 mmol). The mixture was stirred at room until no more acid was detected by LCMS. To the mixture was added N¹,N¹-diethylethane-1,2-diamine (22 mL, 0.155 mmol) and stirred at room temperature overnight. The solution was prepared by HPLC to yield 2-chloro-N-(2-(diethylamino)ethyl)-5-(5-((4-oxo-2-thioxothiazolidin-5-ylidene)methyl)furan-2-yl)benzamide. LCMS (M+1=464).

### Example 21

### Synthesis of 2-chloro-N-(2-methoxyethyl)-5-(5-((4-oxo-2-thioxothiazolidin-5-ylidene)methyl)furan-2-yl)benzamide

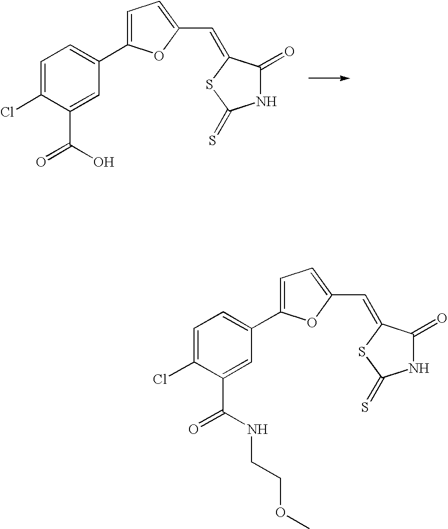

To 2-chloro-5-(5-((4-oxo-2-thioxothiazolidin-5-ylidene)methyl)furan-2-yl)benzoic acid (47 mg, 0.129 mmol) in DMF was added HBTU (73 mg, 0.193 mmol) and DIEA (67 mL, 0.386 mmol). The mixture was stirred at room temperature until no more acid was detected by LCMS. To the mixture was added 2-methoxyethanamine (13 mL, 0.155 mmol) and stirred at room temperature overnight. The solution was prepared by HPLC to yield 2-chloro-N-(2-methoxyethyl)-5-(5-((4-oxo-2-thioxothiazolidin-5-ylidene)methyl)furan-2-yl)benzamide. LCMS (M+1=423).

### Example 22

### Synthesis of 3-(5-((3-methyl-5-oxo-1H-pyrazol-4(5H)-ylidene)methyl)furan-2-yl)benzoic Acid

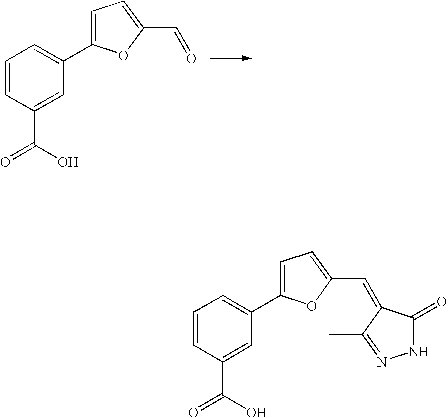

To 3-(5-formylfuran-2-yl)benzoic acid (100 mg, 0.46 mmol) in EtOH was added 3-methyl-1H-pyrazol-5(4H)-one (54 mg, 0.55 mmol) and piperidine (46 μL, 0.46 mmol). The mixture was stirred at room temperature overnight. The solution was then passed through a plug of silica and solvent removed under reduced pressure. The remaining solid was washed with EtOAc to yield 3-(5-((3-methyl-5-oxo-1H-pyrazol-4(5H)-ylidene)methyl)furan-2-yl)benzoic acid. LCMS (M+1=297).

### Example 23

### Synthesis of 3-methyl-4-((5-(3-(4-methyl-1,4-diazepane-1-carbonyl)phenyl)furan-2-yl)methylene)-1H-pyrazol-5(4H)-one

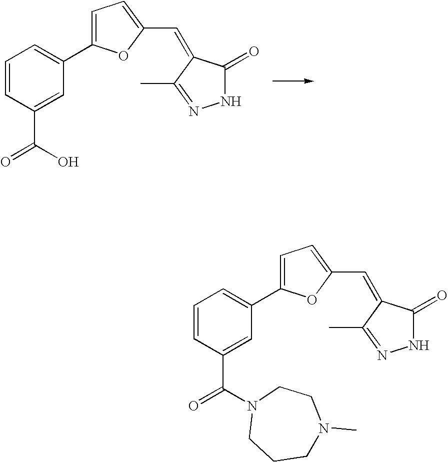

To 3-(5-((3-methyl-5-oxo-1H-pyrazol-4(5H)-ylidene)methyl)furan-2-yl)benzoic acid (42 mg, 0.142 mmol) in DCM was added 1-methylhomopiperazine (70 μL, 0.568 mmol), HOBT (38 mg, 0.284 mmol), EDCI (54 mg, 0.284 mmol), and DIEA (99 μL, 0.568 mmol). The mixture was stirred at room temperature. The solution was then diluted with water and product extracted in DCM. The organic layer was dried over Na₂SO₄ and concentrated under reduced pressure. The resulting solid was prepared by TLC (3% MeOH/DCM) to yield 3-methyl-4-(5-(3-(4-methyl-1,4-diazepane-1-carbonyl)phenyl)furan-2-yl)methylene)-1H-pyrazol-5(4H)-one. LCMS (M+1=393).

### Example 24

### Synthesis of 3-(5-((4-oxo-2-thioxothiazolidin-5-ylidene)methyl)furan-2-yl)benzoic Acid

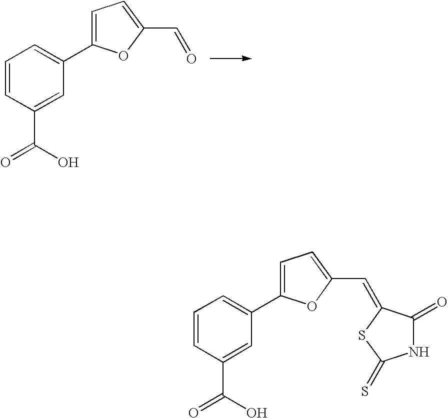

To 3-(5-formylfuran-2-yl)benzoic acid (100 mg, 0.46 mmol) in EtOH was added rhodanine (61 mg, 0.46 mmol) and piperidine (46 mL, 0.46 mmol). The mixture was stirred at room temperature overnight. The solid formed was isolated by filtration and air dried to yield 125 mg 3-(5-((4-oxo-2-thioxothiazolidin-5-ylidene)methyl)furan-2-yl)benzoic acid (82% yield). LCMS (M+1=332).

### Example 25

### (m) Synthesis of 5-((5-(3-(4-methyl-1,4-diazepane-1-carbonyl)phenyl)furan-2-yl)methylene)-2-thioxothiazolidin-4-one

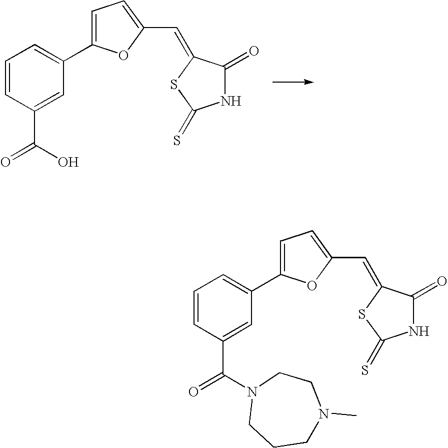

To 3-(5-((4-oxo-2-thioxothiazolidin-5-ylidene)methyl)furan-2-yl)benzoic acid (61 mg, 0.18 mmol) in DCM was added 1-methylhomopiperazine (89 mL, 0.72 mmol), HOBT (49 mg, 0.36 mmol), EDCI (69 mg, 0.36 mmol), and DIEA (126 mL, 0.72 mmol). The mixture was stirred at room temperature. The solution was diluted with water, and the product extracted in DCM. The organic layer was dried over Na₂SO₄ and concentrated under reduced pressure. The resulting solid was prepared by TLC to yield 5-((5-(3-(4-methyl-1,4-diazepane-1-carbonyl)phenyl)furan-2-yl)methylene)-2-thioxothiazolidin-4-one. LCMS (M+1=428).

### Example 26

### (n) Synthesis of 3-(5-((4-oxo-2-thioxothiazolidin-5-ylidene)methyl)furan-2-yl)-N-(2-(pyrrolidin-1-yl)ethyl)benzamide

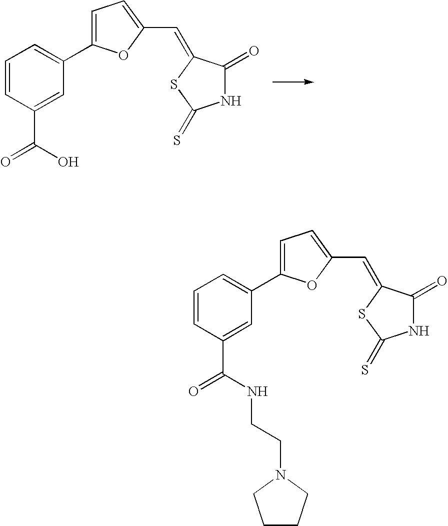

To 3-(5-((4-oxo-2-thioxothiazolidin-5-ylidene)methyl)furan-2-yl)benzoic acid (61 mg, 0.18 mmol) in DCM was added 2-(pyrrolidin-1-yl)ethanamine (90 mL, 0.72 mmol), HOBT (49 mg, 0.36 mmol), EDCI (69 mg, 0.36 mmol), and DIEA (126 mL, 0.72 mmol). The mixture was stirred at room temperature. The solution was diluted with water, and the product extracted in DCM. The organic layer was dried over Na₂SO₄ and concentrated under reduced pressure. The resulting solid was prepared by TLC to yield 3-(5-((4-oxo-2-thioxothiazolidin-5-ylidene)methyl)furan-2-yl)-N-(2-(pyrrolidin-1-yl)ethyl)benzamide. LCMS (M+1=428).

### Example 27

### Synthesis of 4-(5-((2,4-dioxothiazolidin-5-ylidene)methyl)furan-2-yl)benzoic Acid

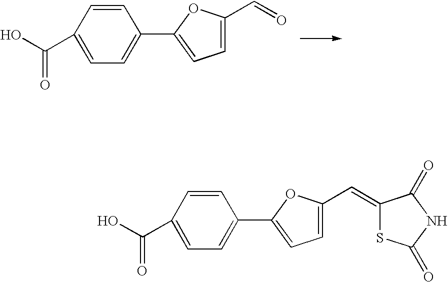

To 4-(5-formylfuran-2-yl)benzoic acid (217 mg, 1 mmol) in 7 mL ETOH was added thiazolidine-2,4-dione (117 mg, 1 mmol) and piperidine (79 μL). The mixture was stirred at 75° C. overnight. Piperidine (79 μL) was added. The mixture was stirred at 75° C. for 3 more hours, filtered, and washed with EtOH to yield 4-(5-((2,4-dioxothiazolidin-5-ylidene)methyl)furan-2-yl)benzoic acid. LCMS (M+1)=316.

### Example 28

### Synthesis of 4-(5-((2,5-dioxoimidazolidin-4-ylidene)methyl)furan-2-yl)benzoic Acid

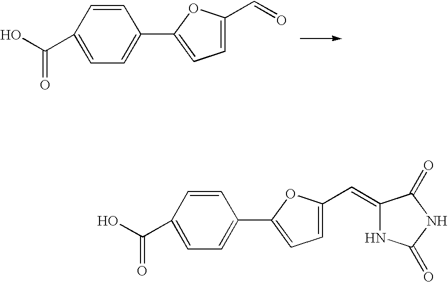

4-(5-((2,5-dioxoimidazolidin-4-ylidene)methyl)furan-2-yl)benzoic acid was prepared as described in the synthesis of 4-(5-((2,4-dioxothiazolidin-5-ylidene)methyl)furan-2-yl)benzoic acid using hydantoin instead of thiazolidine-2,4-dione. LCMS (M+1)=299.

### Example 29

### Synthesis of 4-(5-((3-methyl-2,5-dioxoimidazolidin-4-ylidene)methyl)furan-2-yl)benzoic Acid

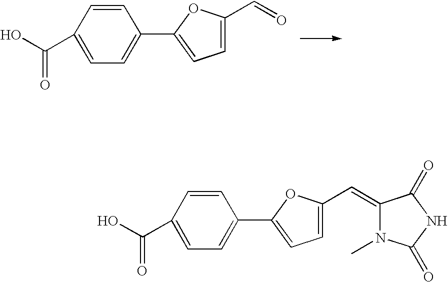

4-(5-((3-methyl-2,5-dioxoimidazolidin-4-ylidene)methyl)furan-2-yl)benzoic acid was prepared as described in the synthesis of 4-(5-((2,4-dioxothiazolidin-5-ylidene)methyl)furan-2-yl)benzoic acid using methyl hydantoin instead of thiazolidine-2,4-dione. LCMS (M+1)=313.

### Example 30

### Synthesis of 3-(5-((2,4-dioxothiazolidin-5-ylidene)methyl)furan-2-yl)benzoic Acid

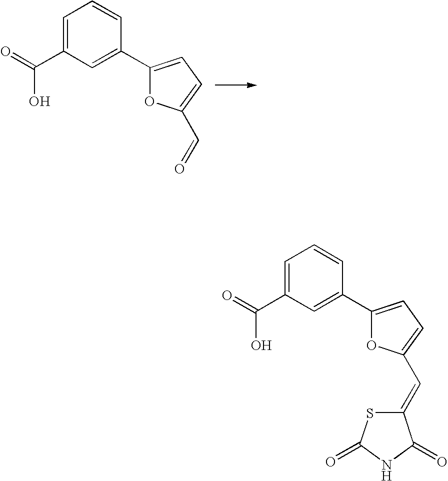

To 3-(5-formylfuran-2-yl)benzoic acid (217 mg, 1 mmol) in 7 mL EtOH was added thiazolidine-2,4-dione (117 mg, 1 mmol) and piperidine (79 μL). The mixture was stirred at 60° C. for 6 hr. NaOH (48 mg) was added. The mixture was stirred for 1 hr, filtered, washed with EtOH and water. The solid was dried overnight. LCMS (M+1)=316.

### Example 31

### Synthesis of 3-(5-((2,5-dioxoimidazolidin-4-ylidene)methyl)furan-2-yl)benzoic Acid

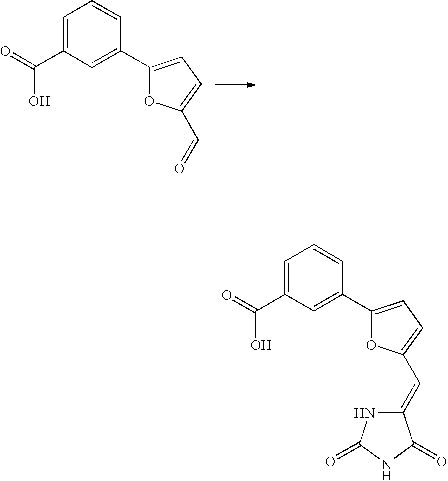

To 3-(5-formylfuran-2-yl)benzoic acid (217 mg, 1 mmol) in 7 mL EtOH was added hydantoin (100 mg, 1 mmol) and piperidine (79 μL). The mixture was stirred at 60° C. overnight, and then heated at 75° C. Hydantoin (100 mg) and piperidine (79 μL) were added. The mixture was stirred at 75° C. overnight. EtOH was evaporated to give a solid that was used in the next step without further purification. LCMS (M+1)=299.

### Example 32

### Synthesis of 3-(5-((3-methyl-2,5-dioxoimidazolidin-4-ylidene)methyl)furan-2-yl)benzoic Acid

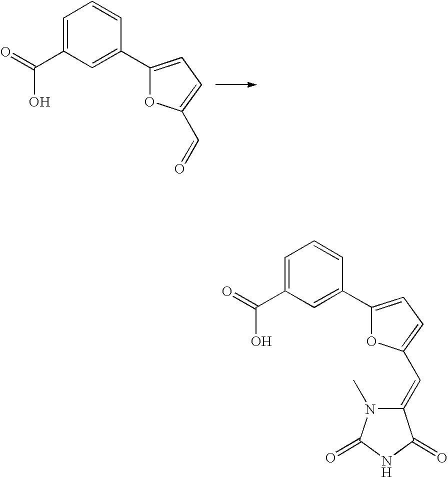

3-(5-((3-methyl-2,5-dioxoimidazolidin-4-ylidene)methyl)furan-2-yl)benzoic acid was prepared as described in the synthesis of 3-(5-((2,5-dioxoimidazolidin-4-ylidene)methyl)furan-2-yl)benzoic acid using methyl hydantoin instead of thiazolidine-2,4-dione. LCMS (M+1)=313.

### Example 33

### Synthesis of 5-((5-(4-(4-methyl-1,4-diazepane-1-carbonyl)phenyl)furan-2-yl)methylene)-2-thioxothiazolidin-4-one

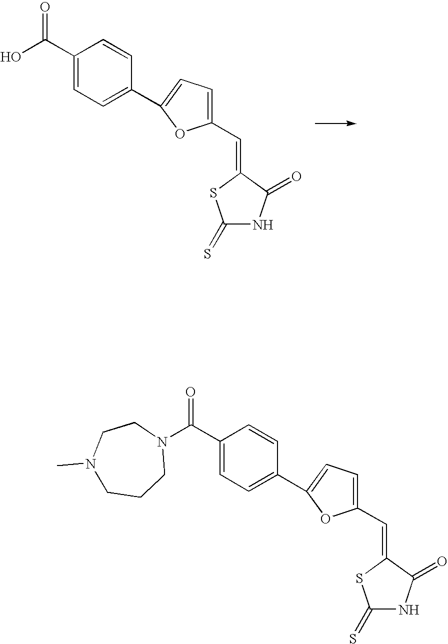

To 4-(5-((4-oxo-2-thioxothiazolidin-5-ylidene)methylifuran-2-yl)benzoic acid (60 mg, 0.181 mmol) in 1 mL DMF/3 mL DCM was added N-methylhomopiperazine (90 μL, 0.725 mmol), HOBt (49 mg, 0.362 mmol) and EDCI (70 mg, 0.362 mmol). The mixture was sonicated until dissolution and DIEA (126 μL, 0.725 mmol) was added. The mixture was stirred at R.T. Water was added and the organic layer was dried with Na₂SO₄ and concentrated under vacuum. The residue obtained was purified by preparative TLC (DCM/MeOH 1%) and the product isolated was purified a second time by HPLC to give 5-((5-(4-(4-methyl-1,4-diazepane-1-carbonyliphenyl)furan-2-yl)methylene)-2-thioxothiazolidin-4-one. LCMS (M+1)=428.

### Example 34

### Synthesis of 5-((5-(4-(4-methylpiperazine-1-carbonyl)phenyl)furan-2-yl)methylene)-2-thioxothiazolidin-4-one

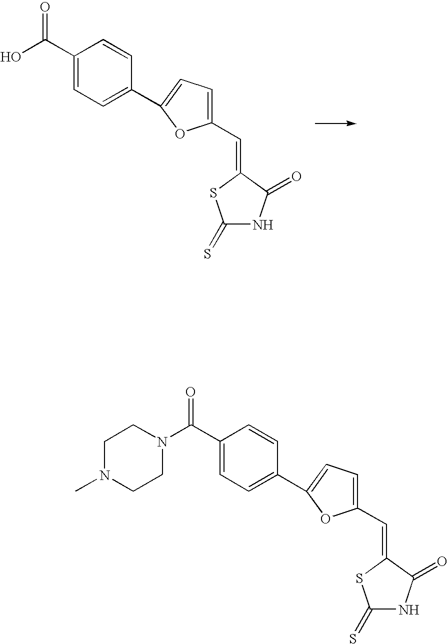

To 4-(5-((4-oxo-2-thioxothiazolidin-5-ylidene)methyl)furan-2-yl)benzoic acid (18 mg, 0.054 mmol) in 1 mL DMF/3 mL DCM was added 1-methylpiperazine (20 μL, 0.217 mmol), HOBt (15 mg, 0.108 mmol) and EDCI (21 mg, 0.108 mmol). The mixture was sonicated until dissolution and DIEA (38 μL, 0.217 mmol) was added. The mixture was stirred at R.T. Water was added the organic layer was dried with Na₂SO₄, concentrated and purified by HPLC to give 5-((5-(4-(4-methylpiperazine-1-carbonyl)phenyl)furan-2-yl)methylene)-2-thioxothiazolidin-4-one. LCMS (M+1)=414.

### Example 35

### Synthesis of 5-((5-(4-(4-(2-aminoethyl)piperazine-1-carbonyl)phenyl)furan-2-yl)methylene)-2-thioxothiazolidin-4-one

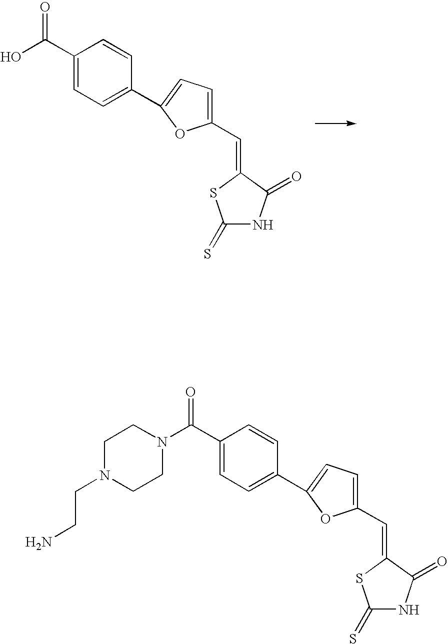

To 4-(5-((4-oxo-2-thioxothiazolidin-5-ylidene)methyl)furan-2-yl)benzoic acid (18 mg, 0.054 mmol) in 1 mL DMF/3 mL DCM was added 2-(piperazin-1-yl)ethanamine (29 μL, 0.217 mmol), HOBt (15 mg, 0.108 mmol) and EDCI (21 mg, 0.108 mmol). The mixture was sonicated until dissolution and DIEA (38 μL, 0.217 mmol) was added. The mixture was stirred at R.T. Water was added and the organic layer was dried with Na₂SO₄ and residue purified by HPLC to give product. LCMS (M+1)=443.

### Example 36

### Synthesis of N-(2-(diethylamino)ethyl)-4-(5-((4-oxo-2-thioxothiazolidin-5-ylidene)methyl)furan-2-yl)benzamide

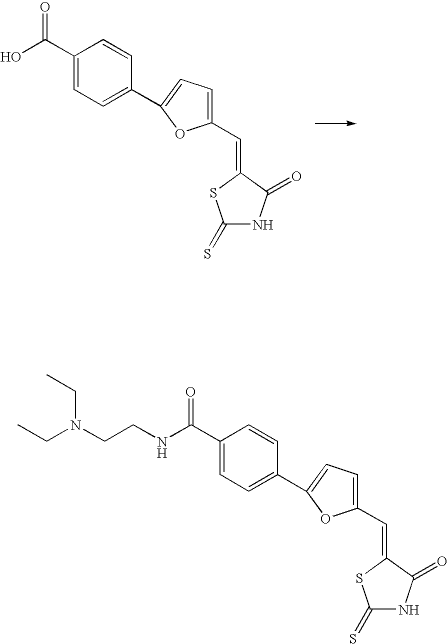

Same procedure as for 5-((5-(4-(4-(2-aminoethyl)piperazine-1-carbonyl)phenyl)furan-2-yl)methylene)-2-thioxothiazolidin-4-one. LCMS (M+1)=430.

### Example 37

### Synthesis of N-((1-ethylpyrrolidin-2-yl)methyl)-4-(5-((4-oxo-2-thioxothiazolidin-5-ylidene)methyl)furan-2-yl)benzamide

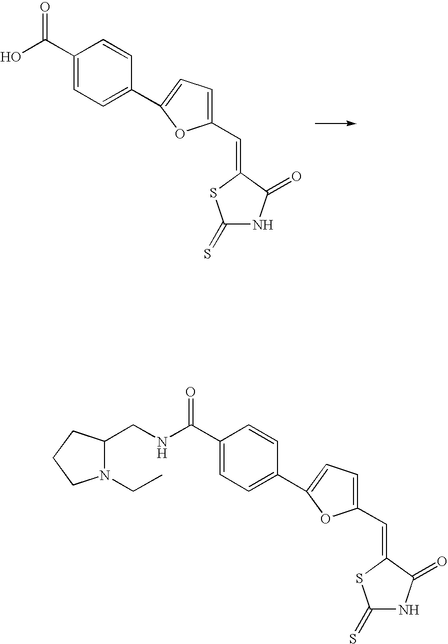

Same procedure as for (Z)-5-((5-(4-(4-(2-aminoethyl)piperazine-1-carbonyl)phenyl)furan-2-yl)methylene)-2-thioxothiazolidin-4-one. LCMS (M+1)=442.

### Example 38

### Synthesis of 5-((5-(4-(4-isopropylpiperazine-1-carbonyl)phenyl)furan-2-yl)methylene)-2-thioxothiazolidin-4-one

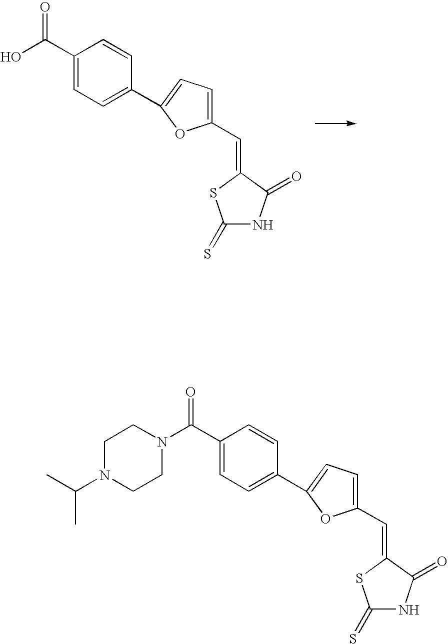

Same procedure as for (Z)-5-((5-(4-(4-(2-aminoethyl)piperazine-1-carbonyl)phenyl)furan-2-yl)methylene)-2-thioxothiazolidin-4-one. LCMS (M+1)=442.

### Example 39

### Synthesis of N-(2-(2-methyl-1H-imidazol-1-yl)ethyl)-4-(5-((4-oxo-2-thioxothiazolidin-5-ylidene)methyl)furan-2-yl)benzamide

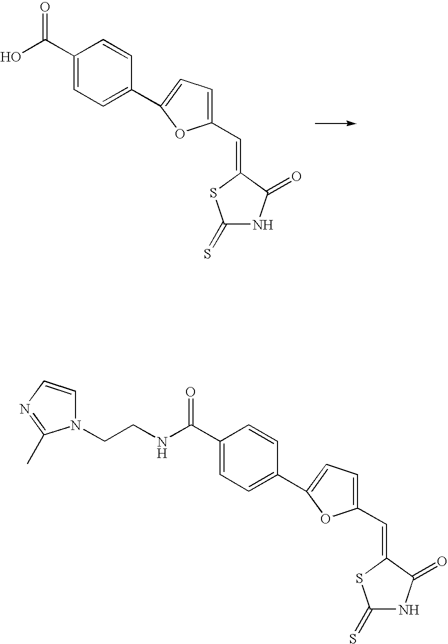

Same procedure as for (Z)-5-((5-(4-(4-(2-aminoethyl)piperazine-1-carbonyl)phenyl)furan-2-yl)methylene)-2-thioxothiazolidin-4-one. LCMS (M+1)=439.

### Example 40

### Synthesis of N-((1-methyl-1H-imidazol-5-yl)methyl)-4-(5-((4-oxo-2-thioxothiazolidin-5-ylidene)methyl)furan-2-yl)benzamide

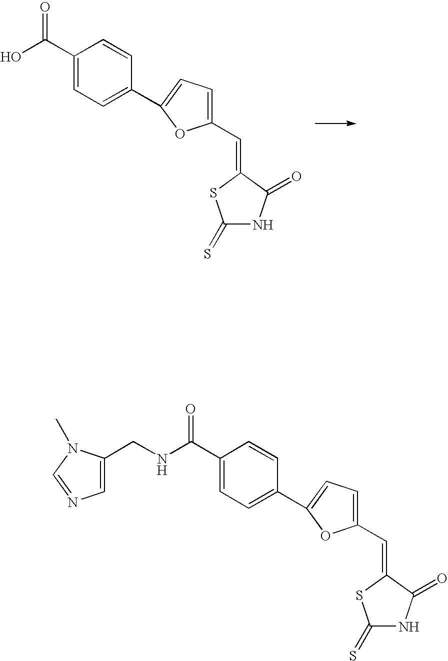

Same procedure as for (Z)-5-((5-(4-(4-(2-aminoethyl)piperazine-1-carbonyl)phenyl)furan-2-yl)methylene)-2-thioxothiazolidin-4-one. LCMS (M+1)=425.

### Example 41

### Synthesis of N-((1-methylpiperidin-2-yl)methyl)-4-(5-((4-oxo-2-thioxothiazolidin-5-ylidene)methyl)furan-2-yl)benzamide

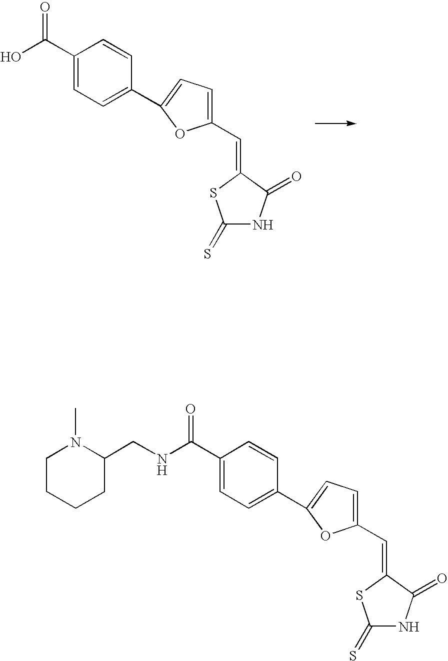

Same procedure as for 5-((5-(4-(4-(2-aminoethyl)piperazine-1-carbonyl)phenyl)furan-2-yl)methylene)-2-thioxothiazolidin-4-one. LCMS (M+1)=442.

### Example 42

### Synthesis of N-methyl-N-(1-methylpyrrolidin-3-yl)-4-(5-((4-oxo-2-thioxothiazolidin-5-ylidene)methyl)furan-2-yl)benzamide

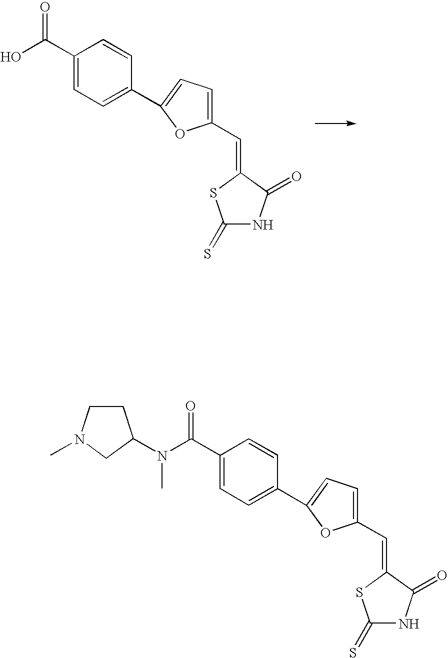

Same procedure as for 5-((5-(4-(4-(2-aminoethyl)piperazine-1-carbonyl)phenyl)furan-2-yl)methylene)-2-thioxothiazolidin-4-one. LCMS (M+1)=428.

### Example 43

### Synthesis of 4-(5-((4-oxo-2-thioxothiazolidin-5-ylidene)methyl)furan-2-yl)-N-(pyridin-2-ylmethyl)benzamide

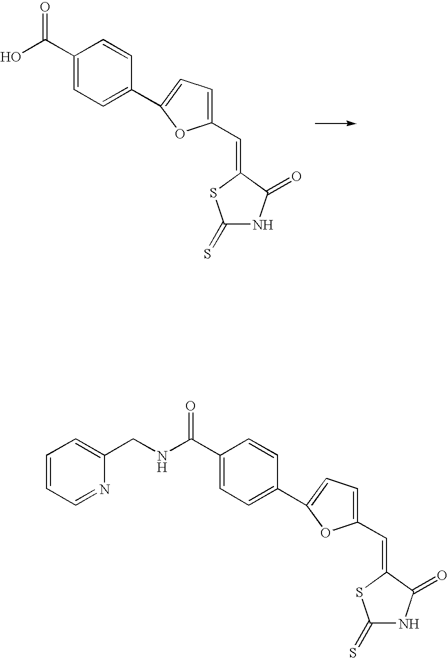

Same procedure as for 5-((5-(4-(4-(2-aminoethyl)piperazine-1-carbonyl)phenyl)furan-2-yl)methylene)-2-thioxothiazolidin-4-one. LCMS (M+1)=422.

### Example 44

### Synthesis of 5-((5-(3-(4-methyl-1,4-diazepane-1-carbonyl)phenyl)furan-2-yl)methylene)thiazolidine-2,4-dione

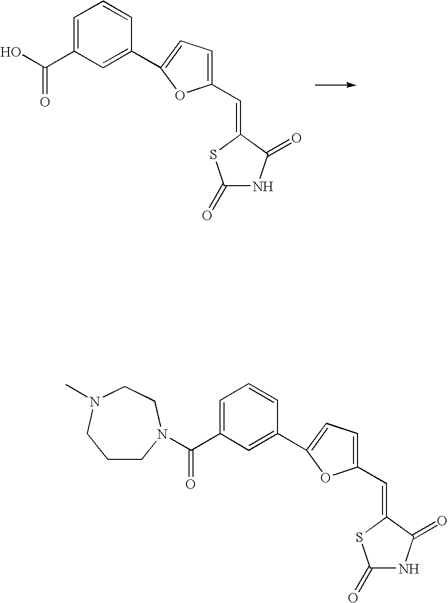

To 3-(5-((2,4-dioxothiazolidin-5-ylidene)methyl)furan-2-yl)benzoic acid (245 mg, 0.77 mmol) in 2.5 mL NMP was added HBTU (436 mg, 1.15 mmol) and DIEA (268 μL, 1.54 mmol). The mixture was stirred until dissolution at R.T. Then N-methyl,homopiperazine (212 μL, 1.71 mmol) was added and 2 hr later, DIEA was added again (268 μL). The mixture was stirred overnight at R.T. Product was isolated by HPLC. LCMS (M+1)=412.

### Example 45

### Synthesis of 1-methyl-5-((5-(3-(4-methyl-1,4-diazepane-1-carbonyl)phenyl)furan-2-yl)methylene)imidazolidine-2,4-dione

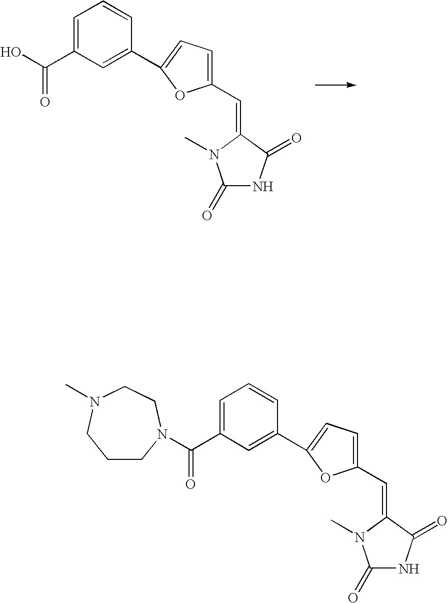

To 3-(5-((2,4-dioxothiazolidin-5-ylidene)methyl)furan-2-yl)benzoic acid (100 mg, 0.32 mmol) in 1.5 mL NMP was added HBTU (182 mg, 0.48 mmol) and DIEA (111 μL, 0.64 mmol). The mixture was stirred until dissolution at R.T. Then N-methyl,homopiperazine (88 μL, 0.705 mmol) was added and the mixture was stirred overnight at R.T. Product was isolated by HPLC. LCMS (M+1)=409.

### Example 46

### Synthesis of 5-((5-(3-(4-methyl-1,4-diazepane-1-carbonyl)phenyl)furan-2-yl)methylene)imidazolidine-2,4-dione

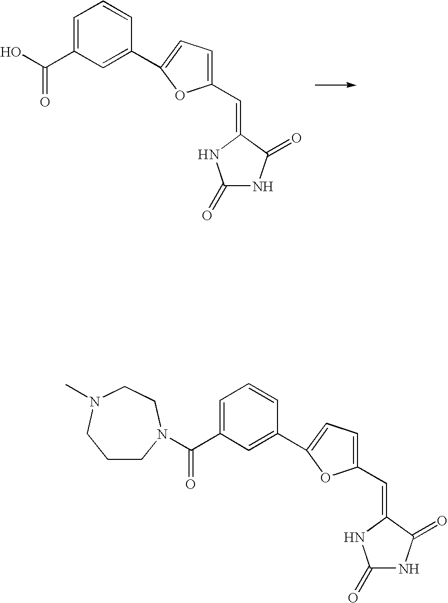

To 3-(5-((2,4-dioxothiazolidin-5-ylidene)methyl)furan-2-yl)benzoic acid (90 mg, 0.302 mmol) in 2.5 mL NMP was added HBTU (172 mg, 0.453 mmol) and DIEA (105 μL, 0.604 mmol). The mixture was stirred until dissolution at R.T. for 20 minutes. Then N-methylhomopiperazine (83 μL, 0.664 mmol) was added and the mixture was stirred overnight at R.T. Product was obtained by purification by HPLC. LCMS (M+1)=395.

### Example 47

### Synthesis of N-ethyl-3-(5-((4-oxo-2-thioxothiazolidin-5-ylidene)methyl)furan-2-yl)benzamide

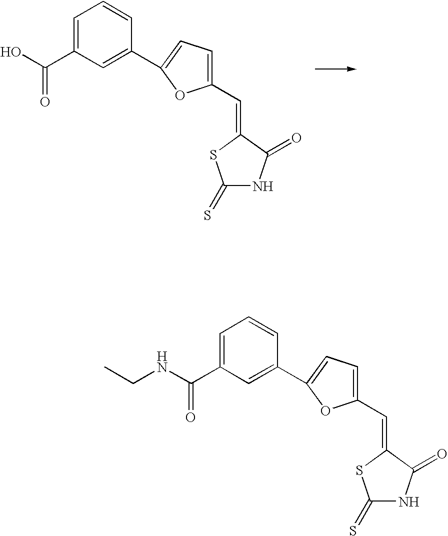

Same procedure as 5-((5-(3-(4-methyl-1,4-diazepane-1-carbonyl)phenyl)furan-2-yl)methylene)thiazolidine-2,4-dione. LCMS (M+1)=359.

### Example 48

### Synthesis of N-(2-methoxyethyl)-3-(5-((4-oxo-2-thioxothiazolidin-5-ylidene)methyl)furan-2-yl)benzamide

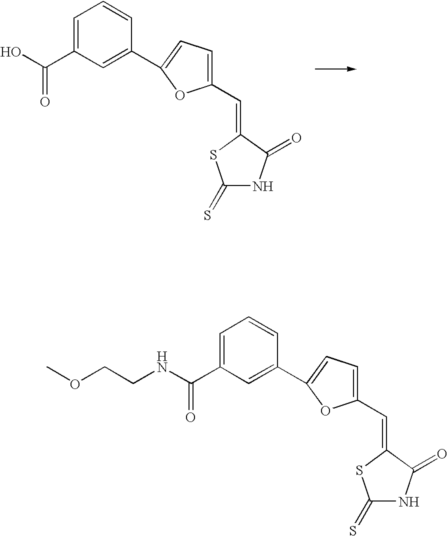

Same procedure as 5-((5-(3-(4-methyl-1,4-diazepane-1-carbonyl)phenyl)furan-2-yl)methylene)thiazolidine-2,4-dione. LCMS (M+1)=389.

### Example 49

### Synthesis of (S)-5-((5-(3-(3-fluoropyrrolidine-1-carbonybphenyl)furan-2-yl)methylene)-2-thioxothiazolidin-4-one

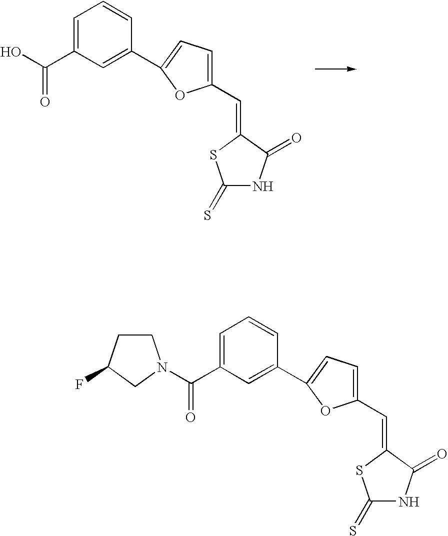

Same procedure as 5-((5-(3-(4-methyl-1,4-diazepane-1-carbonyl)phenyl)furan-2-yl)methylene)thiazolidine-2,4-dione. LCMS (M+1)=403.

### Example 50

### Synthesis of (S)-5-(5-(3-(2-(hydroxymethyl)pyrrolidine-1-carbonyl)phenyl)furan-2-yl)methylene)-2-thioxothiazolidin-4-one

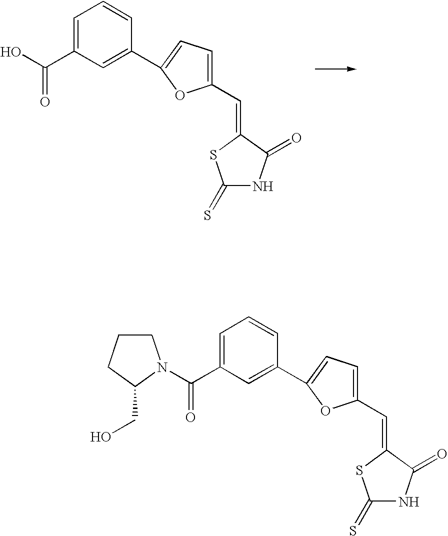

Same procedure as 5-((5-(3-(4-methyl-1,4-diazepane-1-carbonyl)phenyl)furan-2-yl)methylene)thiazolidine-2,4-dione. LCMS (M+1)=415.

### Example 51

### Synthesis of (R)-5-((5-(3-(3-fluoropyrrolidine-1-carbonyl)phenyl)furan-2-yl)methylene)-2-thioxothiazolidin-4-one

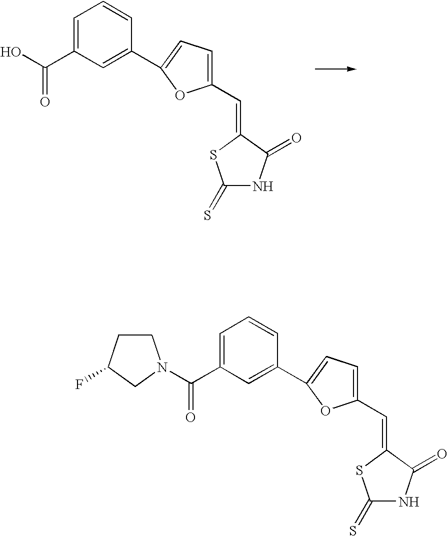

Same procedure as 5-((5-(3-(4-methyl-1,4-diazepane-1-carbonyl)phenyl)furan-2-yl)methylene)thiazolidine-2,4-dione. LCMS (M+1)=403.

### Example 52

### Synthesis of 5-((5-(3-(3-(acetamido)pyrrolidine-1-carbonyl)phenyl)furan-2-yl)methylene)-2-thioxothiazolidin-4-one

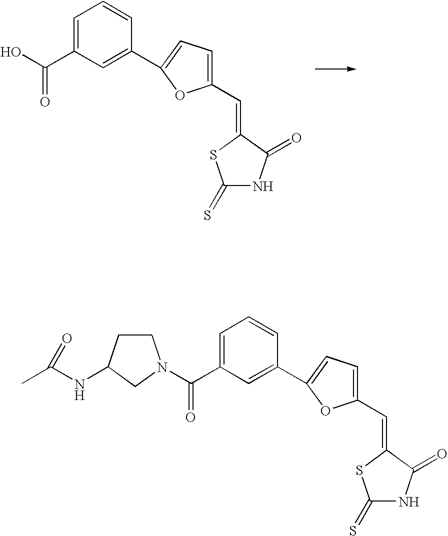

Same procedure as 5-((5-(3-(4-methyl-1,4-diazepane-1-carbonyl)phenyl)furan-2-yl)methylene)thiazolidine-2,4-dione. LCMS (M+1)=442.

### Example 53

### Synthesis of N′-(7-chloroquinolin-4-yl)-3-(5-((4-oxo-2-thioxothiazolidin-5-ylidene)methyl)furan-2-yl)benzohydrazide

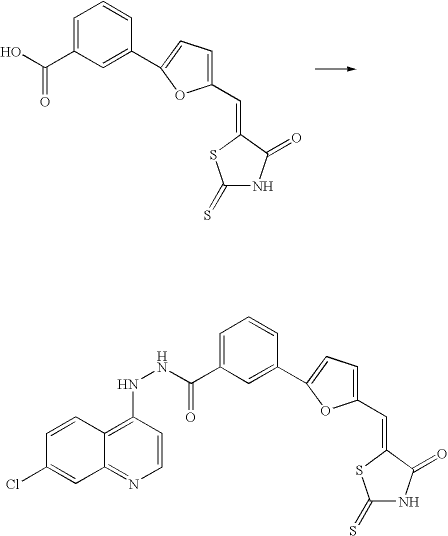

Same procedure as 5-((5-(3-(4-methyl-1,4-diazepane-1-carbonyl)phenyl)furan-2-yl)methylene)thiazolidine-2,4-dione. LCMS (M+1)=507.

### Example 54

### Synthesis of N′,N′-dimethyl-3-(5-((4-oxo-2-thioxothiazolidin-5-ylidene)methyl)furan-2-yl)benzohydrazide

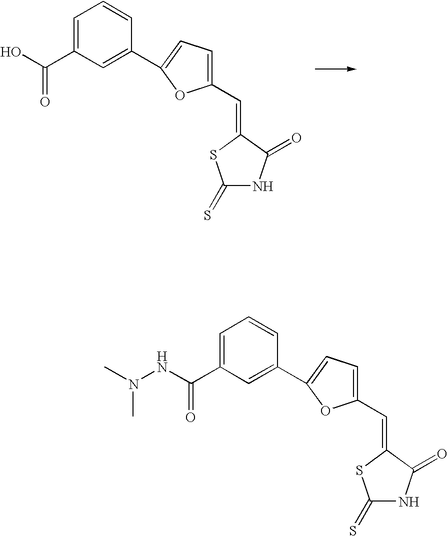

Same procedure as 5-((5-(3-(4-methyl-1,4-diazepane-1-carbonyl)phenyl)furan-2-yl)methylene)thiazolidine-2,4-dione. LCMS (M+1)=374.

### Example 55

### Synthesis of 3-(5-((4-oxo-2-thioxothiazolidin-5-ylidene)methyl)furan-2-yl)-N-(piperidin-1-yl)benzamide

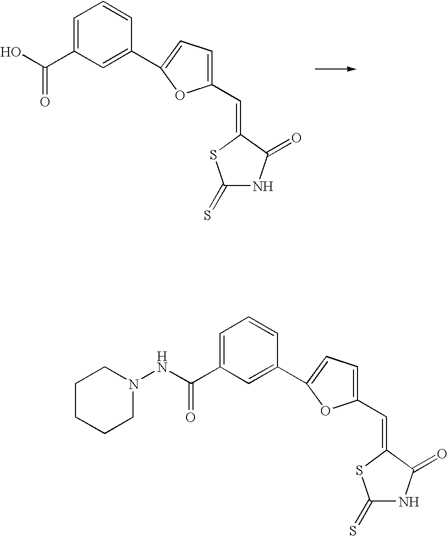

Same procedure as 5-((5-(3-(4-methyl-1,4-diazepane-1-carbonyl)phenyl)furan-2-yl)methylene)thiazolidine-2,4-dione. LCMS (M+1)=400.

### Example 56

### Synthesis of 3-(5-((4-oxo-2-thioxothiazolidin-5-ylidene)methyl)furan-2-yl)-N-(4H-1,2,4-triazol-4-yl)benzamide

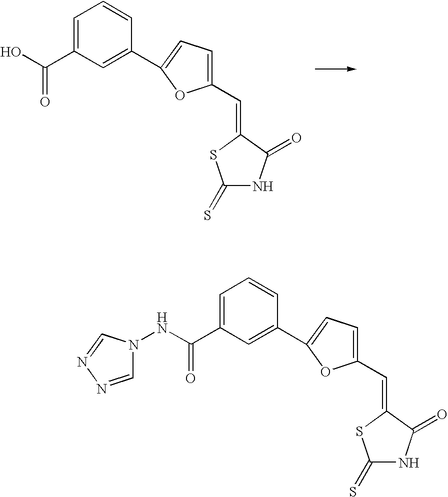

Same procedure as 5-((5-(3-(4-methyl-1,4-diazepane-1-carbonyl)phenyl)furan-2-yl)methylene)thiazolidine-2,4-dione. LCMS (M+1)=398.

### Example 57

### Synthesis of N-(4-(2-hydroxyethyl)piperazin-1-yl)-3-(5-((4-oxo-2-thioxothiazolidin-5-ylidene)methyl)furan-2-yl)benzamide

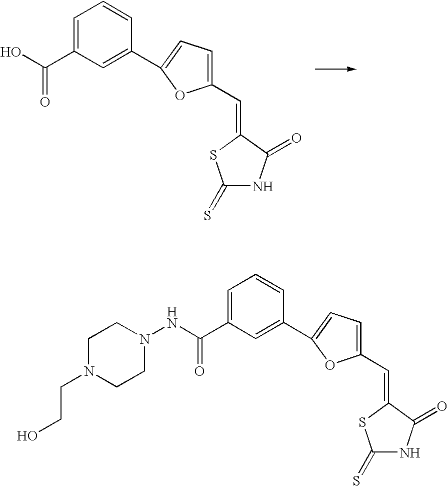

Same procedure as 5-((5-(3-(4-methyl-1,4-diazepane-1-carbonyl)phenyl)furan-2-yl)methylene)thiazolidine-2,4-dione. LCMS (M+1)=459.

### Example 58

### Synthesis of 5-((5-(4-(4-methyl-1,4-diazepane-1-carbonyl)phenyl)furan-2-yl)methylene)thiazolidine-2,4-dione

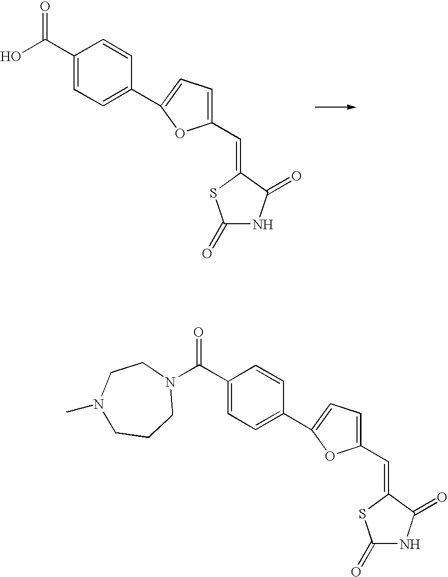

To 4-(5-((2,4-dioxothiazolidin-5-ylidene)methyl)furan-2-yl)benzoic acid (240 mg, 0.77 mmol) in 1.5 mL NMP was added HBTU (436 mg, 1.15 mmol) and DIEA (268 μL, 1.54 mmol). The mixture was stirred until dissolution at R.T. Then N-methyl,homopiperazine (212 μL, 1.71 mmol) was added and the mixture was stirred overnight at R.T. Water was added and the solid formed was isolated by filtration and air dried. LCMS (M+1)=412.

### Example 59

### Synthesis of 1-methyl-5-((5-(4-(4-methyl-1,4-diazepane-1-carbonyl)phenyl)furan-2-yl)methylene)imidazolidine-2,4-dione

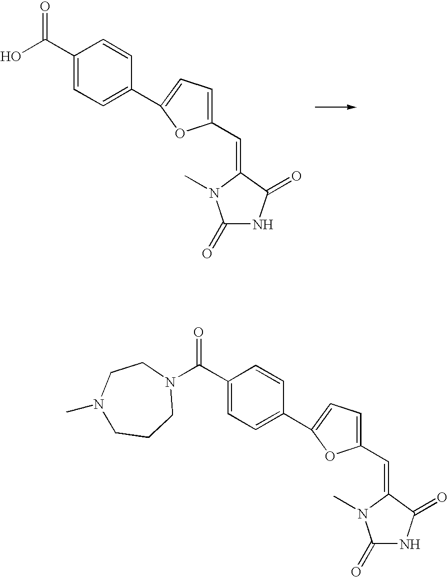

To 4-(5-((3-methyl-2,5-dioxoimidazolidin-4-ylidene)methyl)furan-2-yl)benzoic acid (100 mg, 0.32 mmol) in 1.5 mL NMP was added HBTU (182 mg, 0.48 mmol) and DIEA (111 μL, 0.64 mmol). The mixture was stirred until dissolution at R.T. Then N-methyl,homopiperazine (88 μL, 0.705 mmol) was added and the mixture was stirred overnight at R.T. The product was purified by HPLC. LCMS (M+1)=409.

### Example 60

### Synthesis of (Z)-5-((5-(4-(4-methyl-1,4-diazepane-1-carbonyl)phenyl)furan-2-yl)methylene)imidazolidine-2,4-dione

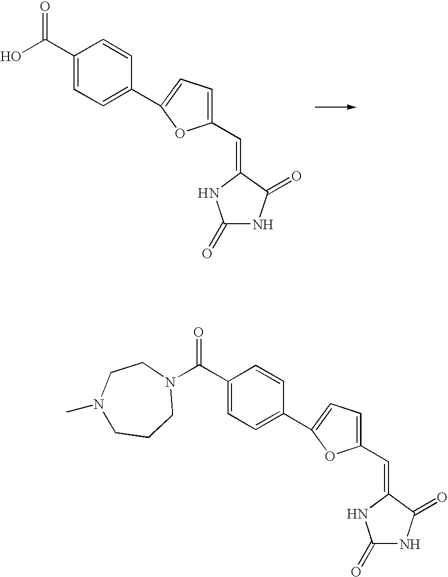

The same procedure as above.

### Example 61

### Synthesis of 3-(5-((4-oxo-2-thioxothiazolidin-5-ylidene)methyl)furan-2-yl)benzamide

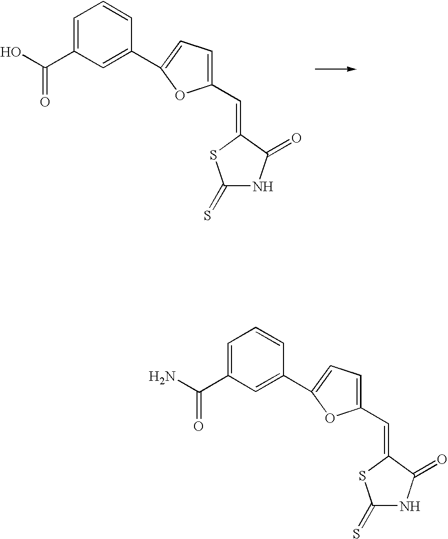

To 3-(5-((4-oxo-2-thioxothiazolidin-5-ylidene)methyl)furan-2-yl)benzoic acid (50 mg, 0.151 mmol)) in 1 mL NMP was added ammonium chloride (64 mg, 1.21 mmol), HOBt (41 mg, 0.302 mmol) and DIEA (105 μL, 0.604 mmol). The mixture was stirred at R.T. during 40 minutes until dissolution, then at 80° C. overnight. Product was purified by HPLC to give an orange solid. EtOAc was added and the solid formed was isolated by filtration. LCMS (M+1)=331.

### Example 62

### Synthesis of 5-((5-phenylfuran-2-yl)methylene)-2-thioxothiazolidin-4-one

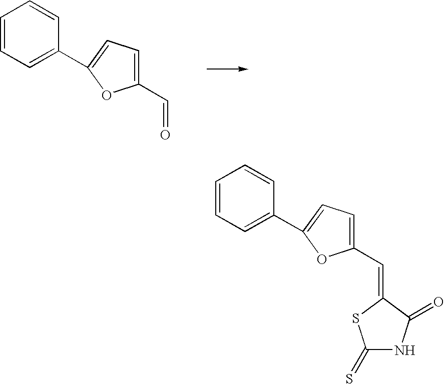

To 5-phenylfuran-2-carbaldehyde (74 μL, 0.376 mmol) in 3 mL EtOH, was added rhodanine (50 mg, 0.376 mmol) and piperidine (37 μL, 0.376 mmol). The mixture was stirred at R.T. The product was purified by HPLC. LCMS (M+1)=288.

### Example 63

### Synthesis of methyl 3-(5-((4-oxo-2-thioxothiazolidin-5-ylidene)methyl)furan-2-yl)benzoate

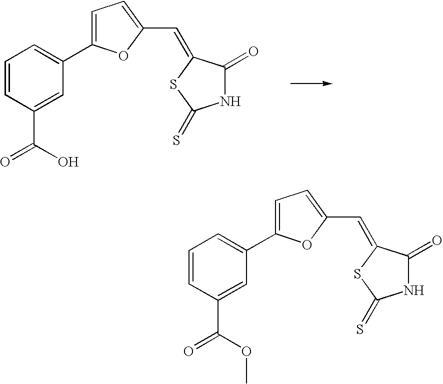

To 3-(5-((4-oxo-2-thioxothiazolidin-5-ylidene)methylifuran-2-yl)benzoic acid (50 mg, 0.151 mmol) in DCM was added (COCl)₂ (39 mL, 0.453 mmol) and drops of DMF. The mixture was allowed to stir at room temperature. When quenched with methanol, LCMS shows methyl ester. Methanol was added to reaction flask and stir at room temperature. The solid formed was isolated by filtration purified by HPLC to yield methyl 3-(5-((4-oxo-2-thioxothiazolidin-5-ylidene)methyl)furan-2-yl)benzoate. LCMS (M+1=346).

### Example 64

### Synthesis of 2-(5-((4-oxo-2-thioxothiazolidin-5-ylidene)methyl)furan-2-yl)benzoic Acid

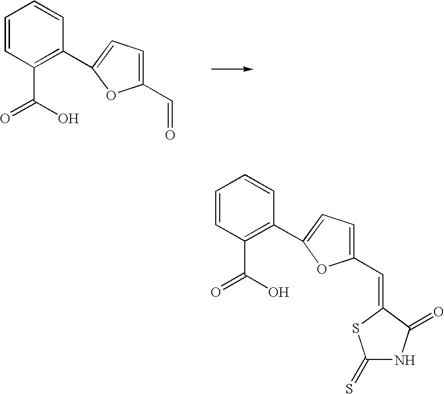

To 2-(5-formylfuran-2-yl)benzoic acid (80 mg, 0.370 mmol) in 1.5 mL EtOH was added rhodanine (50 mg, 0.370 mmol) and piperidine (37 μL, 0.370 mmol).The mixture was stirred at R.T. The solid formed was isolated by filtration and air dried to give product. LCMS (M+1)=332.

### Example 65

### Synthesis of 5-((5-(2-(4-methyl-1,4-diazepane-1-carbonyl)phenyl)furan-2-yl)methylene)-2-thioxothiazolidin-4-one

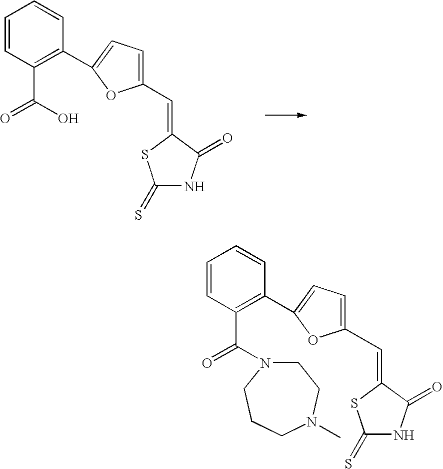

To 2-(5-((4-oxo-2-thioxothiazolidin-5-ylidene)methyl)furan-2-yl)benzoic acid (75 mg, 0.226 mmol) in 1 mL NMP was added HBTU (128 mg, 0.339 mmol) and DIEA (79 μL, 0.452 mmol). The mixture was stirred until dissolution and N-methylhomopiperazine (28 μL, 0.226 mmol) was added. The mixture was stirred at R.T. until no more starting material was detected by LCMS. The product was purified by HPLC. LCMS (M+1)=428.

### Example 66

### Pyrazine Analogs Synthesis: General Procedure

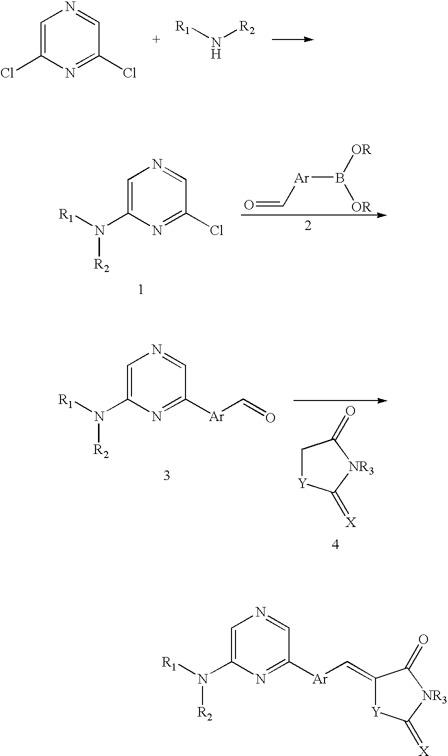

2,6-dichloropyrazine can be reacted in an appropriate solvent at reflux or at an appropriate temperature with amines to give intermediate 1. The Suzuki type reaction of compound 1 with aryl substituted boronic acids or esters can lead to compound 3. Reagent 4 can undergo a Knoevenagel type condensation with intermediate 3 in the presence of piperidine in an appropriate solvent to give the desired product.

### Example 67

### Synthesis of 1-(6-chloropyrazin-2-yl)-4-methyl-1,4-diazepane

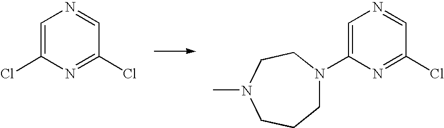

To 2,6-dichloropyrazine (309 mg, 2.07 mmol) in 1 mL acetonitrile was added N-methylhomopiperazine (645 μL). The mixture was heated in microwave at 80° C. for 10 min Water was added and product extracted with DCM, dried with sodium sulfate and concentrated under vacuum.

### Example 68

### Synthesis of 3-(6-(4-methyl-1,4-diazepan-1-yl)pyrazin-2-yl)benzaldehyde

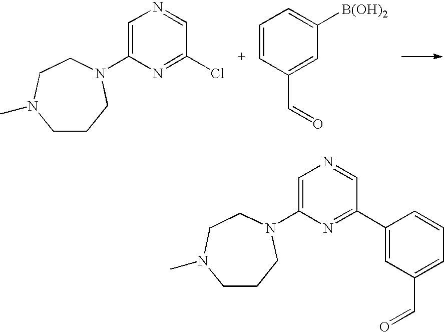

To 1-(6-chloropyrazin-2-yl)-4-methyl-1,4-diazepane (98.4 mg, 0.43 mmol) in dioxane-water (1 mL-100 μL) was added 3-formylphenylboronic acid (123.7 mg, 0.82 mmol) and 185.3 mg cesium carbonate. The mixture was degassed during 10 minutes with nitrogen and 16 mg of PdCl₂(dppf)₂ was added. The mixture was heated in microwave at 100° C. for 15 min water was added and the product extracted with DCM, washed with brine, dried with sodium sulfate and concentrated under vacuum. Residue obtained was dissolved in ethyl acetate and precipitate formed was filtered and product was obtained after removal of ethyl acetate. The product was used in the next step without further purification.

### Example 69

### Synthesis of 5-(3-(6-(4-methyl-1,4-diazepan-1-yl)pyrazin-2-yl)benzylidene)thiazolidine-2,4-dione

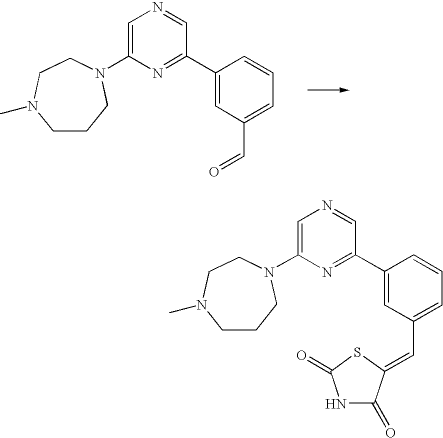

To 3-(6-(4-methyl-1,4-diazepan-1-yl)pyrazin-2-yl)benzaldehyde (30 mg, 0.10 mmol) in 0.5 mL EtOH was added 2,4-thiazolidinedione (11.86 mg, 0.10 mmol) and piperidine (10.13 μL). The mixture was heated at 70° C. overnight. Water was added and product was extracted with DCM, dried with sodium sulfate and concentrated under vacuum. To the residue obtained was added methanol and precipitate was isolated by filtration to give product. LCMS (M+1)=396.

### Example 70

### Synthesis of 5-(3-(6-(4-methyl-1,4-diazepan-1-yl)pyrazin-2-yl)benzylidene)-2-thioxothiazolidin-4-one

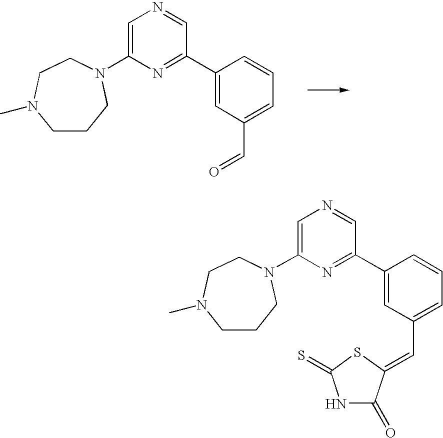

To 3-(6-(4-methyl-1,4-diazepan-1-yl)pyrazin-2-yl)benzaldehyde (100 mg, 0.33 mmol) in 1 mL EtOH was added Rhodanine (44 mg) and piperidine (33 μL). The mixture was heated at 70° C. for 2 hr. Water was added and product was extracted with DCM, dried with sodium sulfate and concentrated under vacuum. To the residue obtained was added methanol and precipitate was isolated by filtration to give product. LCMS (M+1)=412.

### Example 71

### Synthesis of 5-(6-(4-methyl-1,4-diazepan-1-yl)pyrazin-2-yl)furan-2-carbaldehyde

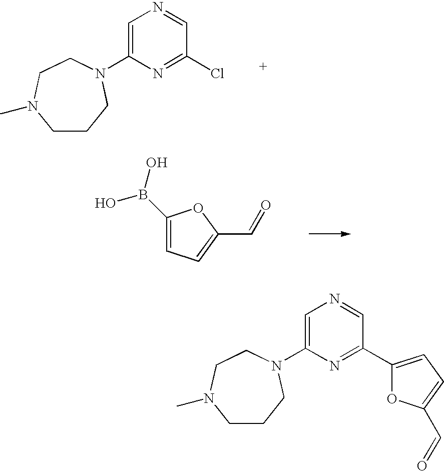

To 1-(6-chloropyrazin-2-yl)-4-methyl-1,4-diazepane (300 mg, 1.33 mmol) in 4 ml Dioxane/water (0.05%) was added 5-formylfuran-2-ylboronic acid (278 mg, 1.99 mmol) and cesium carbonate (1301 mg, 3.99 mmol). The mixture was degassed under nitrogen during 10 minutes. PdCl2(dppf)2 (49 mg, 0.066 mmol) was then added. The mixture was put in the microwave at 100° C. for 10 minutes. The mixture was heated in microwave at 100° C. for 15 min water was added and the product extracted with DCM, washed with brine, dried with sodium sulfate and concentrated under vacuum. Residue obtained was dissolved in ethyl acetate and precipitate formed was filtered and product was obtained after removal of ethyl acetate. LCMS (M+1)=287.

### Example 72

### Synthesis of 5-((5-(6-(4-methyl-1,4-diazepan-1-yl)pyrazin-2-yl)furan-2-yl)methylene)-2-thioxothiazolidin-4-one

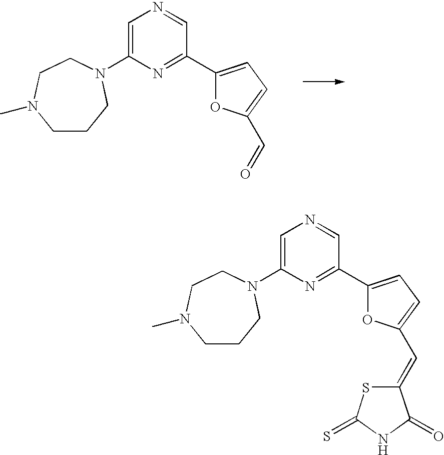

To 5-(6-(4-methyl-1,4-diazepan-1-yl)pyrazin-2-yl)furan-2-carbaldehyde (190 mg, 0.664 mmol) in 4 mL EtOH was added rhodanine (90 mg, 0.664 mmol) and piperidine (66 μL). The mixture was stirred at 70° C. overnight. Water was added and the product was extracted with DCM, dried with Na2SO4 and concentrated under vacuum. MeOH was added to the residue and the precipitate formed was isolated by filtration to give 5-((5-(6-(4-methyl-1,4-diazepan-1-yl)pyrazin-2-yl)furan-2-yl)methylene)-2-thioxothiazolidin-4-one. LCMS (M+1)=402.

### Example 73

### Synthesis of 5-((5-(6-(4-methyl-1,4-diazepan-1-yl)pyrazin-2-yl)furan-2-yl)methylene)-thiazolidine-2,4-dione

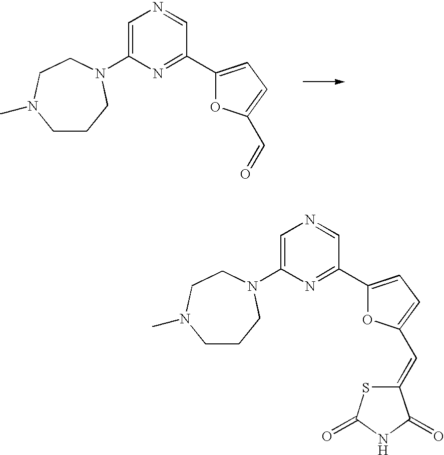

To 5-(6-(4-methyl-1,4-diazepan-1-yl)pyrazin-2-yl)furan-2-carbaldehyde (90 mg, 0.314 mmol) in 4 mL EtOH was added thiazolidine-2,4-dione (37 mg, 0.314 mmol) and piperidine (31 μL). The mixture was stirred at 70° C. overnight. Water was added and the product was extracted with DCM, dried on Na2SO4 and concentrated under vacuum. The residue obtained was purified by HPLC. LCMS (M+1)=386.

### Example 74

### Synthesis of 2-fluoro-4-(6-(4-methyl-1,4-diazepan-1-yl)pyrazin-2-yl)benzaldehyde

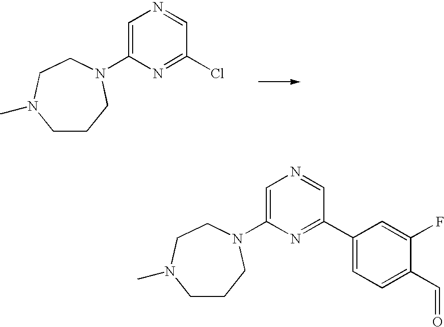

To 1-(6-chloropyrazin-2-yl)-4-methyl-1,4-diazepane (80 mg, 0.35 mmol) in dioxane/water (20:1) was added Cs₂CO₃ (346 mg, 1.06 mmol) and 3-fluoro-4-formylphenylboronic acid (89 mg, 0.53 mmol). The mixture was degassed under N₂ then PdCl₂dppf (13 mg, 0.02 mmol) was added and the solution was microwaved 1 hour at 110° C. The mixture was then diluted with water and insolubilities filtered off. The filtrate was then partitioned between water and DCM. The organic layer was prepared by TLC (1% MeOH/DCM+Et₃N) to yield 51 mg (46% yield) of 2-fluoro-4-(6-(4-methyl-1,4-diazepan-1-yl)pyrazin-2-yl)benzaldehyde. LCMS (M+1=315).

### Example 75

### Synthesis of 5-(2-fluoro-4-(6-(4-methyl-1,4-diazepan-1-yl)pyrazin-2-yl)benzylidene)-2-thioxothiazolidin-4-one

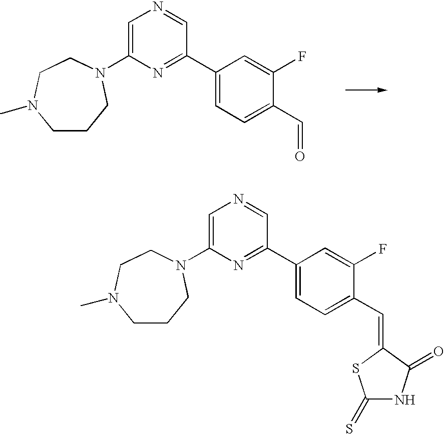

To 2-fluoro-4-(6-(4-methyl-1,4-diazepan-1-yl)pyrazin-2-yl)benzaldehyde (51 mg, 0.16 mmol) in EtOH was added piperidine (16 uL, 0.16 mmol) and 2-thioxothiazolidin-4-one (22 mg, 0.16 mmol). The mixture was stirred at room temperature 72 hours. Precipitate was filtered and dried to yield (Z)-5-(2-fluoro-4-(6-(4-methyl-1,4-diazepan-1-yl)pyrazin-2-yl)benzylidene)-2-thioxothiazolidin-4-one. LCMS (M+1=430).

### Example 76

### Synthesis of 4-fluoro-3-(6-(4-methyl-1,4-diazepan-1-yl)pyrazin-2-yl)benzaldehyde

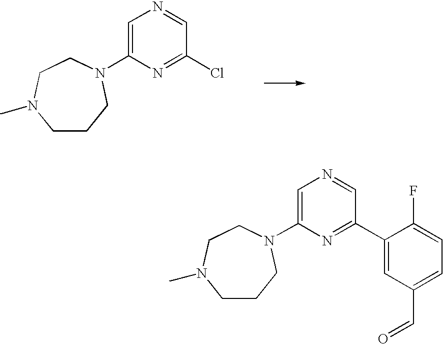

Same procedure as Example 74, but with 2-fluoro-5-formylphenylboronic acid. Yield 44 mg (40% yield) of 4-fluoro-3-(6-(4-methyl-1,4-diazepan-1-yl)pyrazin-2-yl)benzaldehyde. LCMS (M+1=315).

### Example 77

### Synthesis of 5-(4-fluoro-3-(6-(4-methyl-1,4-diazepan-1-yl)pyrazin-2-yl)benzylidene)-2-thioxothiazolidin-4-one

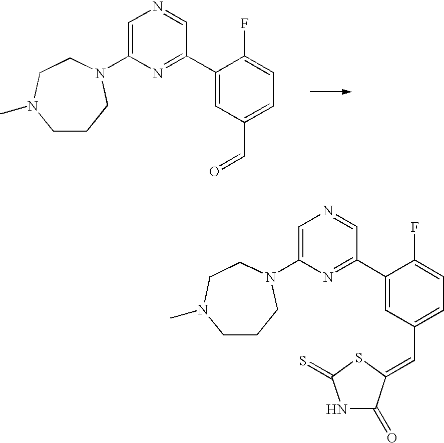

Same procedure as Example 75. Yield (Z)-5-(4-fluoro-3-(6-(4-methyl-1,4-diazepan-1-yl)pyrazin-2-yl)benzylidene)-2-thioxothiazolidin-4-one. LCMS (M+1=430).

### Example 78

### Synthesis of 4-chloro-3-(6-(4-methyl-1,4-diazepan-1-yl)pyrazin-2-yl)benzaldehyde

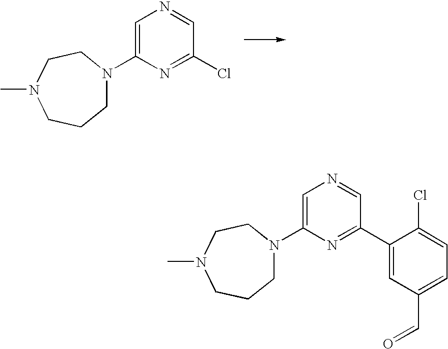

Same procedure as Example 74, but with 2-chloro-5-formylphenylboronic acid. Yield 31 mg (27% yield) of 4-chloro-3-(6-(4-methyl-1,4-diazepan-1-yl)pyrazin-2-yl)benzaldehyde. LCMS (M+1=331).

### Example 79

### Synthesis of 5-(4-chloro-3-(6-(4-methyl-1,4-diazepan-1-yl)pyrazin-2-yl)benzylidene)thiazolidine-2,4-dione

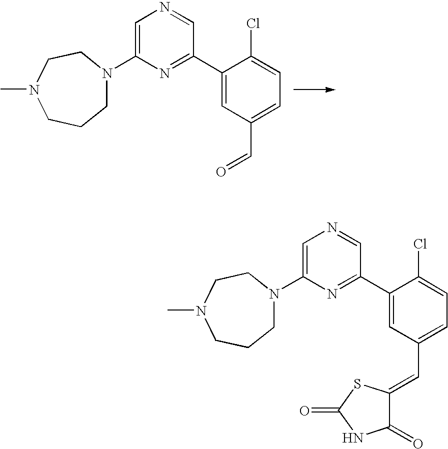

Same procedure as Example 75 but with thiazolidine-2,4-dione. Yield (Z)-5-(4-chloro-3-(6-(4-methyl-1,4-diazepan-1-yl)pyrazin-2-yl)benzylidene)thiazolidine-2,4-dione. LCMS (M+1=430).

### Example 80

### Synthesis of 5-(2-fluoro-4-(6-(4-methyl-1,4-diazepan-1-yl)pyrazin-2-yl)benzylidene)thiazolidine-2,4-dione

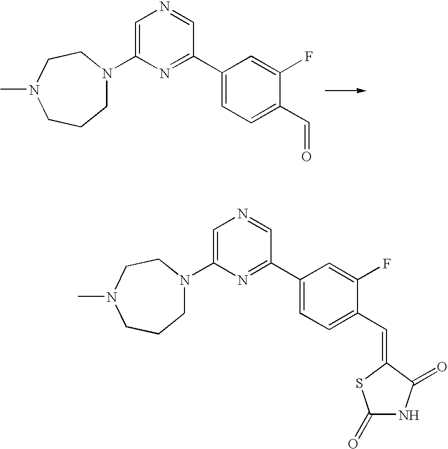

Same procedure as Example 75 but with thiazolidine-2,4-dione. Yield (Z)-5-(2-fluoro-4-(6-(4-methyl-1,4-diazepan-1-yl)pyrazin-2-yl)benzylidene)thiazolidine-2,4-dione. LCMS (M+1=414).

### Example 81

### Synthesis of 5-(6-(4-methyl-1,4-diazepan-1-yl)pyrazin-2-yl)nicotinaldehyde

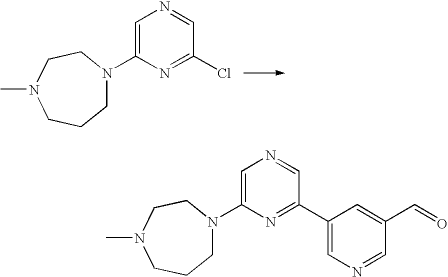

To 1-(6-chloropyrazin-2-yl)-4-methyl-1,4-diazepane (200 mg, 0.89 mmol) in dioxane/water (25:1) was added Cs₂CO₃ (865 mg, 2.66 mmol) and 5-formylpyridin-3-ylboronic acid (309 mg, 1.33 mmol). The mixture was degassed under N₂ then PdCl₂dppf (32 mg, 0.04 mmol) was added and the solution was microwaved 1 hour at 110° C. The mixture was then partitioned between water and DCM. The product was extracted into the organic layer, dried over Na₂SO₄, then concentrated under reduced pressure to yield 5-(6-(4-methyl-1,4-diazepan-1-yl)pyrazin-2-yl)nicotinaldehyde. LCMS (M+1=298).

### Example 82

### Synthesis of 5-((5-(6-(4-methyl-1,4-diazepan-1-yl)pyrazin-2-yl)pyridin-3-yl)methylene)-2-thioxothiazolidin-4-one

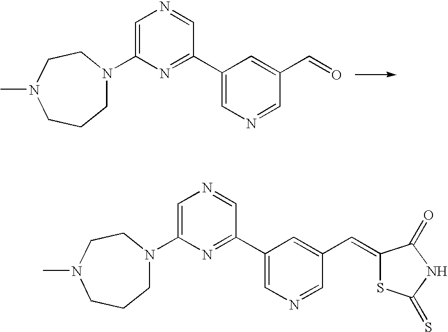

Same procedure as Example 75. Yield (Z)-5-((5-(6-(4-methyl-1,4-diazepan-1-yl)pyrazin-2-yl)pyridin-3-yl)methylene)-2-thioxothiazolidin-4-one. LCMS (M+1=413).

### Example 83

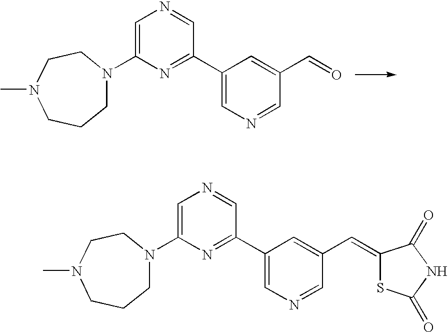

Same procedure as Example 75 but with thiazolidine-2,4-dione. Yield (Z)-5-((5-(6-(4-methyl-1,4-diazepan-1-yl)pyrazin-2-yl)pyridin-3-yl)methylene)thiazolidine-2,4-dione. LCMS (M+1=397).

### Example 84

### Synthesis of 2-methyl-5-(6-(4-methyl-1,4-diazepan-1-yl)pyrazin-2-yl)benzaldehyde

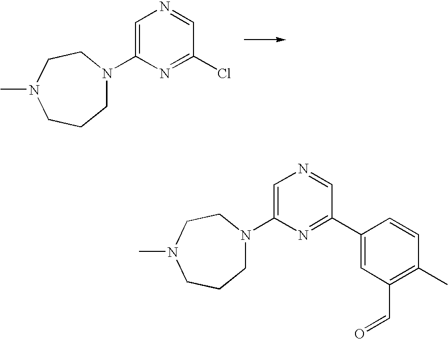

Same procedure as Example 81 but with 3-formyl-4-methylphenylboronic acid. Yield 2-methyl-5-(6-(4-methyl-1,4-diazepan-1-yl)pyrazin-2-yl)benzaldehyde. LCMS (M+1=311).

### Example 85

### Synthesis of 5-(2-methyl-5-(6-(4-methyl-1,4-diazepan-1-yl)pyrazin-2-yl)benzylidene)-2-thioxothiazolidin-4-one

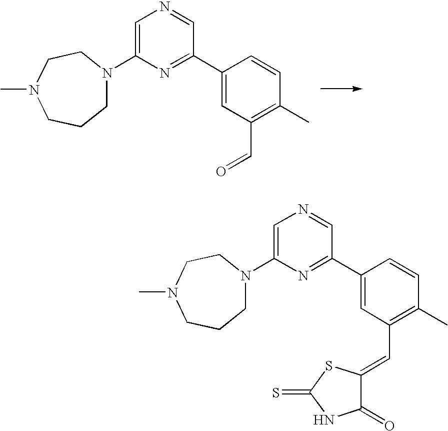

Same procedure as Example 75. Yield (Z)-5-(2-methyl-5-(6-(4-methyl-1,4-diazepan-1-yl)pyrazin-2-yl)benzylidene)-2-thioxothiazolidin-4-one. LCMS (M+1=426).

### Example 86

### Synthesis of 5-(2-methyl-5-(6-(4-methyl-1,4-diazepan-1-yl)pyrazin-2-yl)benzylidene)thiazolidine-2,4-dione

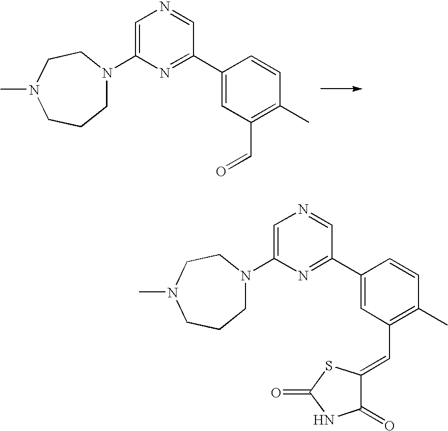

Same procedure as Example 75 but with thiazolidine-2,4-dione. Yield (Z)-5-(2-methyl-5-(6-(4-methyl-1,4-diazepan-1-yl)pyrazin-2-yl)benzylidene)thiazolidine-2,4-dione. LCMS (M+1=410).

### Example 87

### Synthesis of 5-(3-(6-(4-(cyclopropanecarbonyl)-1,4-diazepan-1-yl)pyrazin-2-yl)-4-fluorobenzylidene)thiazolidine-2,4-dione

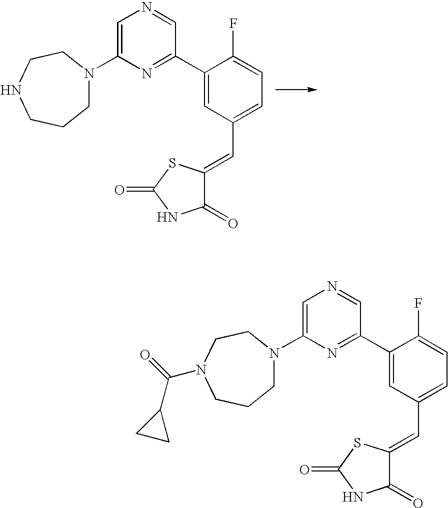

To (Z)-5-(3-(6-(1,4-diazepan-1-yl)pyrazin-2-yl)-4-fluorobenzylidene)thiazolidine-2,4-dione (20 mg, 0.05 mmol) in DCM was added Et₃N (7 uL, 0.05 mmol) and cyclopropanecarbonyl chloride (5 mg, 0.05 mmol). The mixture was stirred at room temperature overnight. DCM was removed under reduced pressure, and the mixture was redissolved in MeOH/DMSO. Prep by HPLC yield (Z)-5-(3-(6-(4-(cyclopropanecarbonyl)-1,4-diazepan-1-yl)pyrazin-2-yl)-4-fluorobenzylidene)thiazolidine-2,4-dione. LCMS (M+1=468).

### Example 88

### Synthesis of ethyl 4-(6-(5-((2,4-dioxothiazolidin-5-ylidene)methyl)-2-fluorophenyl)pyrazin-2-yl)-1,4-diazepane-1-carboxylate

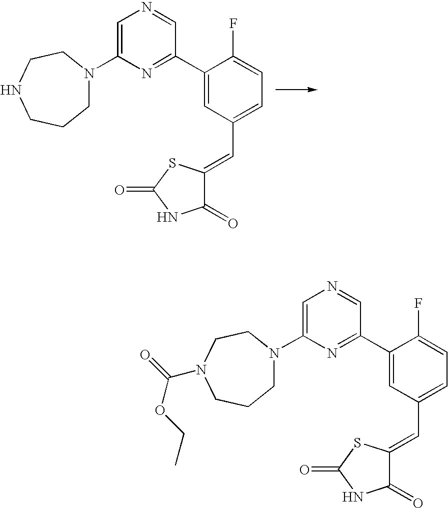

Same procedure as Example 87 but with ethyl carbonochloridate. Yield (Z)-ethyl 4-(6-(5-((2,4-dioxothiazolidin-5-ylidene)methyl)-2-fluorophenyl)pyrazin-2-yl)-1,4-diazepane-1-carboxylate. LCMS (M+1=472).

### Example 89

### Synthesis of 5-(4-fluoro-3-(6-(4-pivaloyl-1,4-diazepan-1-yl)pyrazin-2-yl)benzylidene)thiazolidine-2,4-dione

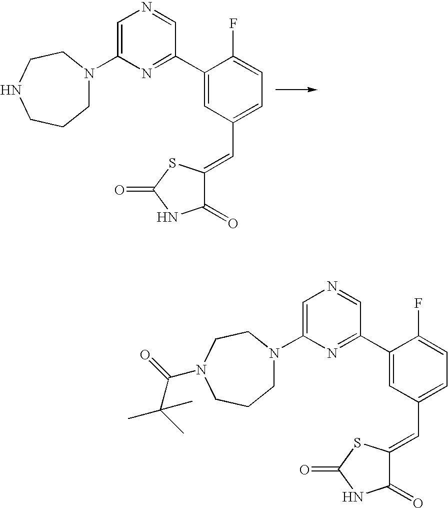

Same procedure as Example 87 but with pivaloyl chloride. Yield (Z)-5-(4-fluoro-3-(6-(4-pivaloyl-1,4-diazepan-1-yl)pyrazin-2-yl)benzylidene)thiazolidine-2,4-dione. LCMS (M+1=484).

### Example 90

### Synthesis of 5-(3-(6-(4-(3,3-dimethylbutanoyl)-1,4-diazepan-1-yl)pyrazin-2-yl)-4-fluorobenzylidene)thiazolidine-2,4-dione

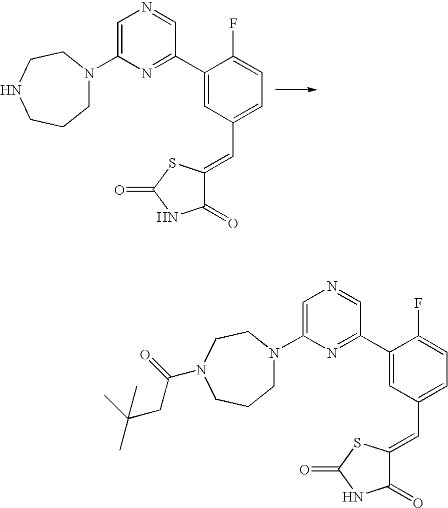

Same procedure as Example 87 but with 3,3-dimethylbutanoyl chloride. Yield (Z)-5-(3-(6-(4-(3,3-dimethylbutanoyl)-1,4-diazepan-1-yl)pyrazin-2-yl)-4-fluorobenzylidene)thiazolidine-2,4-dione. LCMS (M+1=498).

### Example 91

### Synthesis of 5-(4-fluoro-3-(6-(4-(2-phenylacetyl)-1,4-diazepan-1-yl)pyrazin-2-yl)benzylidene)thiazolidine-2,4-dione

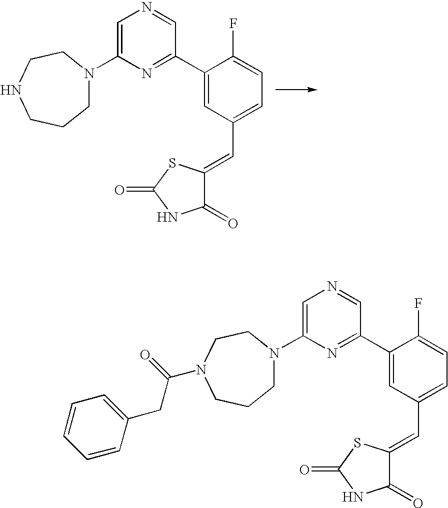

Same procedure as Example 87 but with 2-phenylacetyl chloride. Yield (Z)-5-(4-fluoro-3-(6-(4-(2-phenylacetyl)-1,4-diazepan-1-yl)pyrazin-2-yl)benzylidene)thiazolidine-2,4-dione. LCMS (M+1=518).

### Example 92

### Synthesis of 5-(4-fluoro-3-(6-(4-(2-fluorobenzoyl)-1,4-diazepan-1-yl)pyrazin-2-yl)benzylidene)thiazolidine-2,4-dione

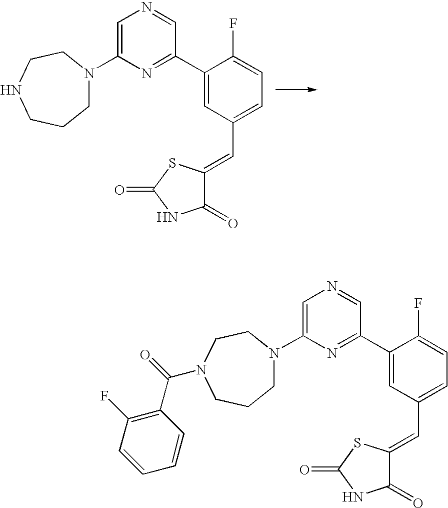

Same procedure as Example 87 but with 2-fluorobenzoyl chloride. Yield (Z)-5-(4-fluoro-3-(6-(4-(2-fluorobenzoyl)-1,4-diazepan-1-yl)pyrazin-2-yl)benzylidene)thiazolidine-2,4-dione. LCMS (M+1=522).

### Example 93

### Synthesis of benzyl 4-(6-(5-((2,4-dioxothiazolidin-5-ylidene)methyl)-2-fluorophenyl)pyrazin-2-yl)-1,4-diazepane-1-carboxylate

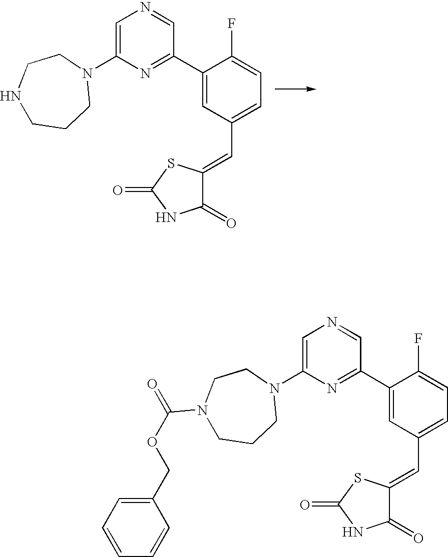

Same procedure as Example 87 but with benzyl carbonochloridate. Yield (Z)-benzyl 446-(5-((2,4-dioxothiazolidin-5-ylidene)methyl)-2-fluorophenyl)pyrazin-2-yl)-1,4-diazepane-1-carboxylate. LCMS (M+1=534).

### Example 94

### Synthesis of phenyl 4-(6-(5-((2,4-dioxothiazolidin-5-ylidene)methyl)-2-fluorophenyl)pyrazin-2-yl)-1,4-diazepane-1-carboxylate

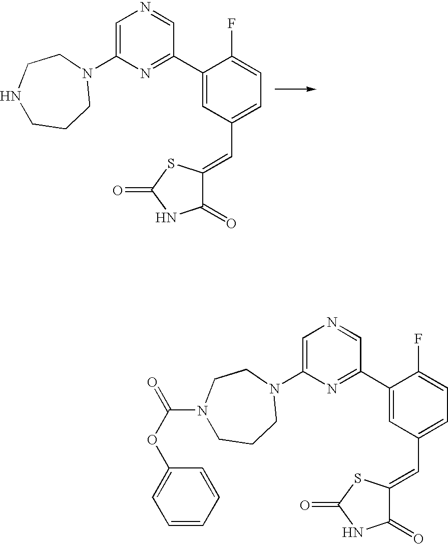

Same procedure as Example 87 but with phenyl carbonochloridate. Yield (Z)-phenyl 446-(5-((2,4-dioxothiazolidin-5-ylidene)methyl)-2-fluorophenyl)pyrazin-2-yl)-1,4-diazepane-1-carboxylate. LCMS (M+1=520).

### Example 95

### Synthesis of methyl 4-(6-(5-((2,4-dioxothiazolidin-5-ylidene)methyl)-2-fluorophenyl)pyrazin-2-yl)-1,4-diazepane-1-carboxylate

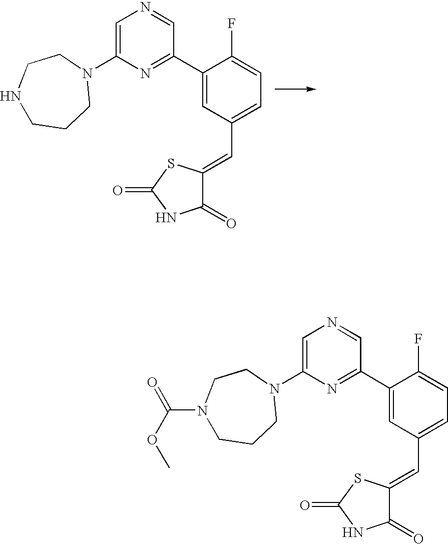

Same procedure as Example 87 but with methyl carbonochloridate. Yield (Z)-methyl 4-(6-(5-((2,4-dioxothiazolidin-5-ylidene)methyl)-2-fluorophenyl)pyrazin-2-yl)-1,4-diazepane-1-carboxylate. LCMS (M+1=458).

### Example 96

### Synthesis of 5-(3-(6-(4-(3,5-dimethoxybenzoyl)-1,4-diazepan-1-yl)pyrazin-2-yl)-4-fluorobenzylidene)thiazolidine-2,4-dione

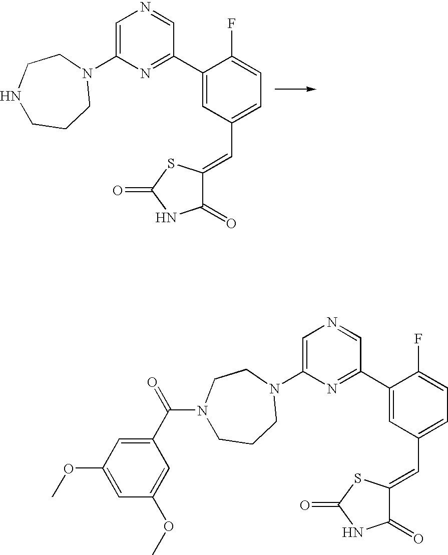

Same procedure as Example 87 but with 3,5-dimethoxybenzoyl chloride. Yield (Z)-5-(3-(6-(4-(3,5-dimethoxybenzoyl)-1,4-diazepan-1-yl)pyrazin-2-yl)-4-fluorobenzylidene)thiazolidine-2,4-dione. LCMS (M+1=564).

### Example 97

### Synthesis of 4-(6-(5-((2,4-dioxothiazolidin-5-ylidene)methyl)-2-fluorophenyl)pyrazin-2-yl)-N-phenyl-1,4-diazepane-1-carboxamide

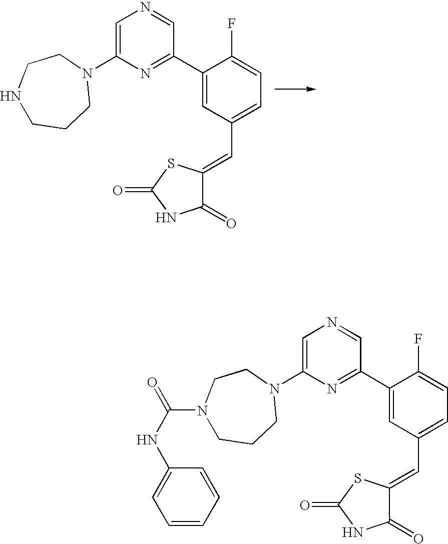

Same procedure as Example 87 but with phenylcarbamic chloride. Yield (Z)-4-(6-(5-((2,4-dioxothiazolidin-5-ylidene)methyl)-2-fluorophenyl)pyrazin-2-yl)-N-phenyl-1,4-diazepane-1-carboxamide LCMS (M+1=519).

### Example 98

### Synthesis of 4-(6-(5-((2,4-dioxothiazolidin-5-ylidene)methyl)-2-fluorophenyl)pyrazin-2-yl)-N-isopropyl-1,4-diazepane-1-carboxamide

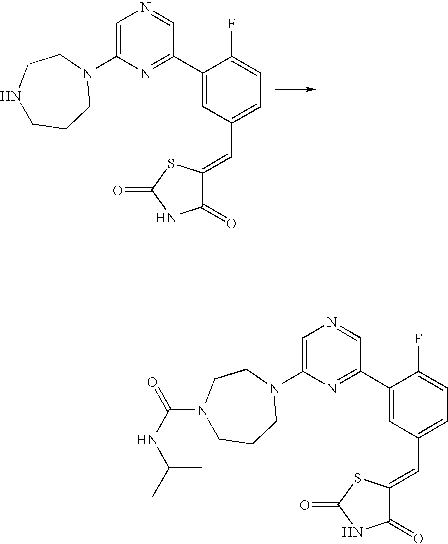

Same procedure as Example 87 but with isopropylcarbamic chloride. Yield (Z)-4-(6-(5-((2,4-dioxothiazolidin-5-ylidene)methyl)-2-fluorophenyl)pyrazin-2-yl)-N-isopropyl-1,4-diazepane-1-carboxamide. LCMS (M+1=485).

### Example 99

### Synthesis of 3-(6-(4-ethyl-1,4-diazepan-1-yl)pyrazin-2-yl)-4-fluorobenzaldehyde

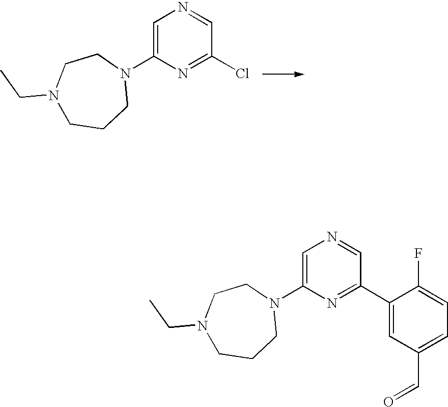

To 1-(6-chloropyrazin-2-yl)-4-ethyl-1,4-diazepane (136 mg, 0.56 mmol) in dioxane/water (20:1) was added Cs₂CO₃ (552 mg) and 2-fluoro-5-formylphenylboronic acid (142 mg, 0.84 mmol). The mixture was degassed under N₂ then PdCl₂dppf (22 mg) was added and the solution was microwaved 10 min at 120° C. The mixture was partitioned between water and DCM. The organic layer was prepared by TLC (5% MeOH/DCM) to yield 3-(6-(4-ethyl-1,4-diazepan-1-yl)pyrazin-2-yl)-4-fluorobenzaldehyde.

### Example 100

### Synthesis of 5-(3-(6-(4-ethyl-1,4-diazepan-1-yl)pyrazin-2-yl)-4-fluorobenzylidene)thiazolidine-2,4-dione

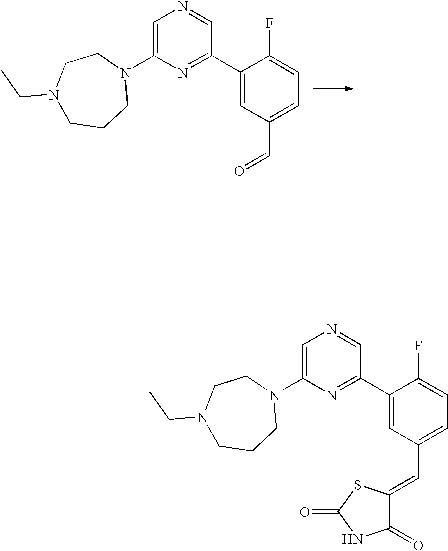

To 3-(6-(4-ethyl-1,4-diazepan-1-yl)pyrazin-2-yl)-4-fluorobenzaldehyde (17 mg, 0.05 mmol) in EtOH was added piperidine (5.7 uL, 0.05 mmol) and 2-thioxothiazolidin-4-one (6.7 mg, 0.05 mmol)

The mixture was stirred at 75° C. over night. Precipitate was filtered and dried to yield 5-(3-(6-(4-ethyl-1,4-diazepan-1-yl)pyrazin-2-yl)-4-fluorobenzylidene)thiazolidine-2,4-dione. LCMS (M+1=428)

### Example 101

### Synthesis of 4-fluoro-3-(6-(4-methyl-1,4-diazepan-1-yl)pyrazin-2-yl)benzaldehyde

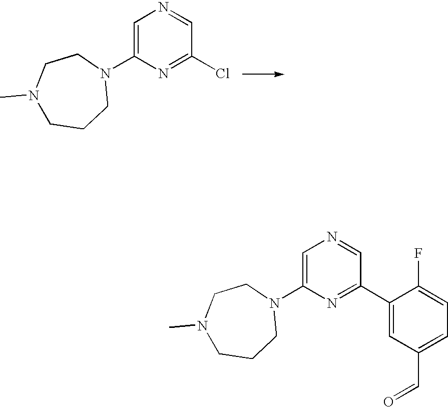

To 1-(6-chloropyrazin-2-yl)-4-methyl-1,4-diazepane (300 mg, 1.32 mmol) in dioxane/water (20:1) was added Cs₂CO₃ (1287 mg) and 2-fluoro-5-formylphenylboronic acid (334 mg, 1.99 mmol)

The mixture was degassed under N₂ then PdCl₂dppf (48 mg) was added and the solution was microwaved 20 min at 120° C. The mixture was partitioned between water and DCM. The organic layer was prepared by TLC (5% MeOH/DCM) to yield 3-(6-(4-methyl-1,4-diazepan-1-yl)pyrazin-2-yl)-4-fluorobenzaldehyde.

### Example 102

### Synthesis of 5-(3-(6-(4-methyl-1,4-diazepan-1-yl)pyrazin-2-yl)-4-fluorobenzylidene)thiazolidine-2,4-dione

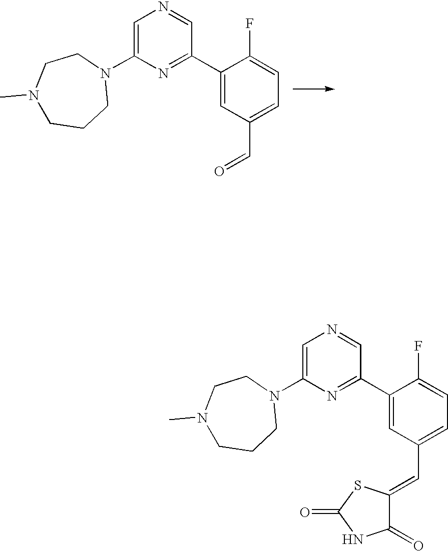

To 3-(6-(4-methyl-1,4-diazepan-1-yl)pyrazin-2-yl)-4-fluorobenzaldehyde (110 mg, 0.37 mmol) in EtOH was added piperidine (36.7 uL, 0.37 mmol) and 2-thioxothiazolidin-4-one (43.5 mg, 0.37 mmol). The mixture was heated over night at 75° C. 2-thioxothiazolidin-4-one (43 mg) and piperidine (36 uL) were added and the mixture heated at 75° C. for 2 days. EtOH was removed and the solid was washed with hot water and EtOH and dried under vacuum to yield 5-(3-(6-(4-methyl-1,4-diazepan-1-yl)pyrazin-2-yl)-4-fluorobenzylidene)thiazolidine-2,4-dione. LCMS (M+1=414).

### Example 103

### Synthesis of 5-(4-fluoro-3-(6-(4-methyl-1,4-diazepan-1-yl)pyrazin-2-yl)benzylidene)-2-thioxothiazolidin-4-one

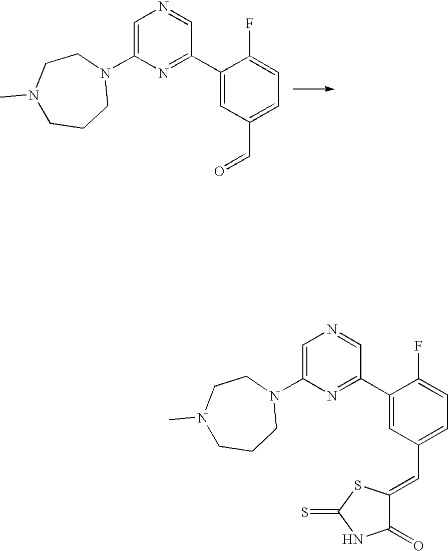

To 3-(6-(4-methyl-1,4-diazepan-1-yl)pyrazin-2-yl)-4-fluorobenzaldehyde (100 mg, 0.34 mmol) in EtOH was added piperidine (33.6 uL, 0.34 mmol) and rhodanine (45 mg, 0.34 mmol). The mixture was stirred at 80° C. over night. Precipitate was filtered and triturated with DCM and dried under vacuum to yield 5-(4-fluoro-3-(6-(4-methyl-1,4-diazepan-1-yl)pyrazin-2-yl)benzylidene)-2-thioxothiazolidin-4-one. LCMS (M+1=430).

### Example 104

### Synthesis of 4-fluoro-3-(6-(4-isopropyl-1,4-diazepan-1-yl)pyrazin-2-yl)benzaldehyde

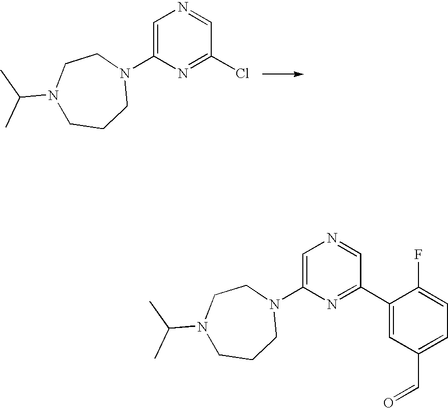

To 1-(6-chloropyrazin-2-yl)-4-isopropyl-1,4-diazepane (300 mg, 1.18 mmol) in Dioxane/water (15% water by volume) was added Cs₂CO₃ (1155 mg, 3.54 mmol) and 2-fluoro-5-formylphenylboronic acid (296 mg, 1.77 mmol). The mixture was degassed under N₂ for 10 minutes, PdCl₂dppf (43 mg, 0.06 mmol) was then added, and the mixture microwaved at 120° C. for 30 minutes. The mixture was then partitioned between water and DCM, product extracted into the organic layer, then dried over MgSO₄. Preparation by TLC (2% MeOH/DCM) yielded 53 mg (13% yield) of 4-fluoro-3-(6-(4-isopropyl-1,4-diazepan-1-yl)pyrazin-2-yl)benzaldehyde as a glassy yellow solid. LCMS (M+1=343).

### Example 105

### Synthesis of 5-(4-fluoro-3-(6-(4-isopropyl-1,4-diazepan-1-yl)pyrazin-2-yl)benzylidene)thiazolidine-2,4-dione

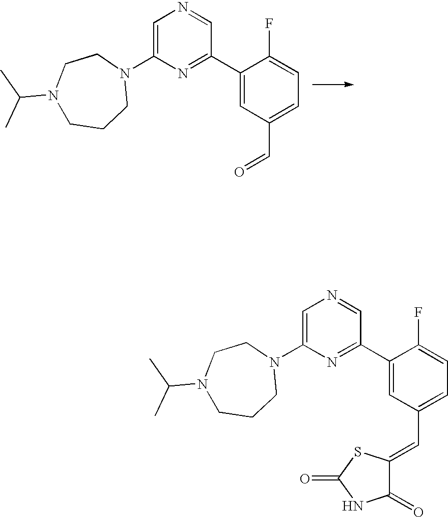

To 3-(6-(4-isopropyl-1,4-diazepan-1-yl)pyrazin-2-yl)-4-fluorobenzaldehyde (53 mg, 0.15 mmol) in EtOH was added piperidine (15.6 uL, 0.15 mmol) and 2-thioxothiazolidin-4-one (18.1 mg, 0.15 mmol). The mixture was stirred at 75° C. over night. Precipitate was filtered and dried to yield 5-(4-fluoro-3-(6-(4-isopropyl-1,4-diazepan-1-yl)pyrazin-2 yl)benzylidene)thiazolidine-2,4-dione. LCMS (M+1=442).

### Example 106

### Synthesis of 5-(4-fluoro-3-(6-(4-isopropyl-1,4-diazepan-1-yl)pyrazin-2-yl)benzylidene)-2-thioxothiazolidin-4-one

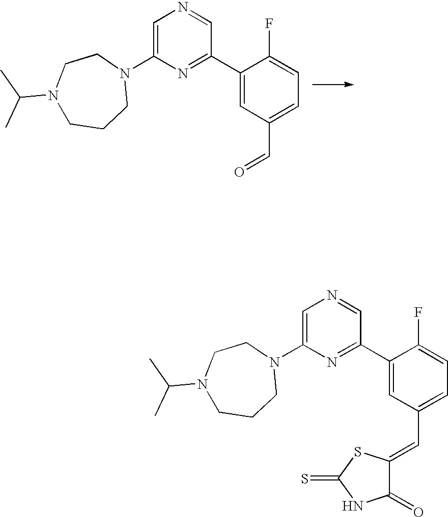

To 3-(6-(4-isopropyl-1,4-diazepan-1-yl)pyrazin-2-yl)-4-fluorobenzaldehyde (20 mg, 0.058 mmol) in EtOH was added piperidine (6 mg, 0.058 mmol) and rhodanine (7.7 mg, 0.058 mmol). The mixture was stirred at 75° C. over night. Precipitate was filtered and purified by prep-HPLC to yield 5-(4-fluoro-3-(6-(4-isopropyl-1,4-diazepan-1-yl)pyrazin-2-yl)benzylidene)-2-thioxothiazolidin-4-one as TFA salt. LCMS (M+1=458).

### Example 107

### Synthesis of 3-methyl-5-(3-(6-(4-methyl-1,4-diazepan-1-yl)pyrazin-2-yl)benzylidene)thiazolidine-2,4-dione

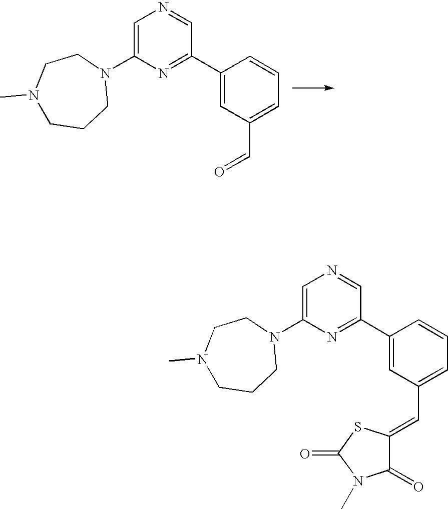

To 3-(6-(4-methyl-1,4-diazepan-1-yl)pyrazin-2-yl)-4-fluorobenzaldehyde (141 mg, 0.52 mmol) in EtOH was added piperidine (52 uL, 0.52 mmol) and 3-methylthiazolidine-2,4-dione (69 mg, 0.52 mmol). The mixture was stirred at room temperature for 3 days. The reaction was heated over night at 75° C. Solid formed was filtered off and filtrate was concentrated under vacuum. A mixture of EtOAc-hexane was added and the solid formed was filtered off and filtrate was concentrated under vacuum. The residue obtained was dissolved in EtOAc and 1 ml of HCl in dioxane (4 N) was added. Precipitate was isolated and triturated with EtOAc-EtOH and solid formed was isolated by filtration and air dried to give 3-methyl-5-(3-(6-(4-methyl-1,4-diazepan-1-yl)pyrazin-2-yl)benzylidene)thiazolidine-2,4-dione as HCl salt. LCMS (M+1=410).

### Example 108

### Synthesis of 1-(3-(6-(4-methyl-1,4-diazepan-1-yl)pyrazin-2-yl)phenyl)ethanone

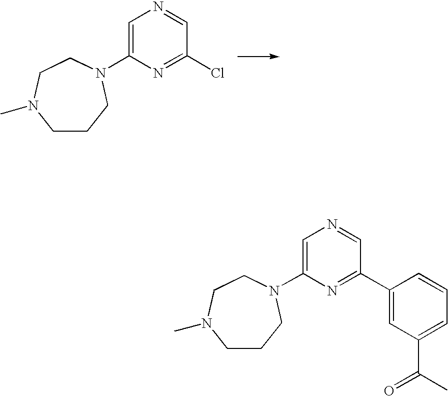

To 1-(6-chloropyrazin-2-yl)-4-methyl-1,4-diazepane (142 mg, 0.626 mmol) in Dioxane/water (15% water by volume) was added Cs₂CO₃ (612 mg) 3-acetylphenylboronic acid (154 mg, 0.939 mmol). The mixture was degassed under N₂ for 10 minutes, PdCl₂dppf (24 mg) was then added, and the mixture microwaved at 120° C. for 30 minutes. The mixture was then partitioned between water and DCM, product extracted into the organic layer, then dried over MgSO₄. Preparation by TLC (2% MeOH/DCM) yielded 1-(3-(6-(4-methyl-1,4-diazepan-1-yl)pyrazin-2-yl)phenyl)ethanone.

### Example 109

### Synthesis of 5-(1-(3-(6-(4-methyl-1,4-diazepan-1-yl)pyrazin-2 yl)phenyl)ethylidene)thiazolidine-2,4-dione

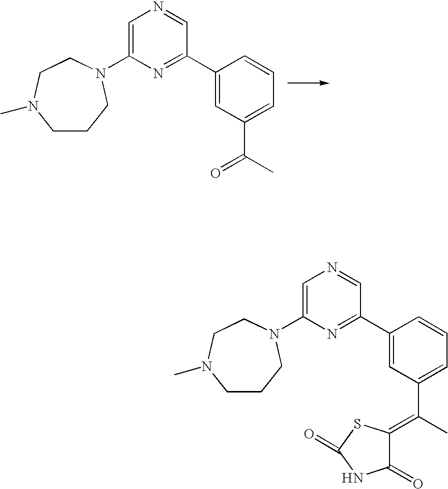

5-(1-(3-(6-(4-methyl-1,4-diazepan-1-yl)pyrazin-2 yl)phenyl)ethylidene)thiazolidine-2,4-dione can be prepared by reacting 1-(3-(6-(4-methyl-1,4-diazepan-1-yl)pyrazin-2-yl)phenyl)ethanone with 2-thioxothiazolidin-4-one in the presence of piperidine at refluxing temperature in toluene.

### Example 110

### Synthesis of Boronic Acid or Boronic Esters Derivatives—General Procedure

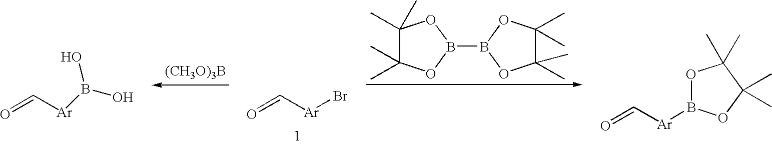

The synthesis of boronic acid or boronic esters derivatives can be carried out via adaptation of procedures found in the literature (WO2005/21552 and Nazarpack-Kandlousy N. J. amer. Chem. Soc. 2000, 122, 3358). The synthesis of boronic acid derivatives can be achieved by treating a compound of the formula 1 (above) with a borate source and a base in suitable solvent and temperature ranging from −78° C. to room temperature. Suitable sources of borate include but are not limited to triisopropylborate and trimethyl borate. Typical bases for use in the reaction include, for example BuLi and tert-Buli. A mixture of tetrahydrofuran and hexane is suitable example of suitable solvent.

The synthesis of boronic ester derivatives can be carried out by the treatment of a compound of formula 1 with bis(pinacolato) diboron and a palladium (0) source in appropriate solvent and temperature. Suitable sources of palladium (0) include but are not limited to palladium (II) acetate and tris(dibenzyldeneacetone) dipalladium (0).

The following are representative examples of commercially available aryl and heteroaryl aldehydes of compound of formula 1.

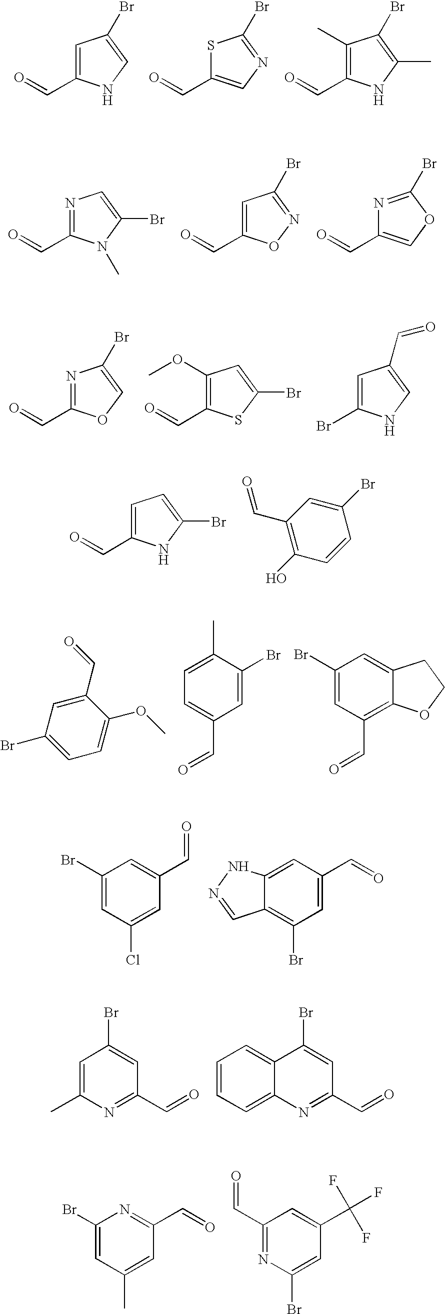

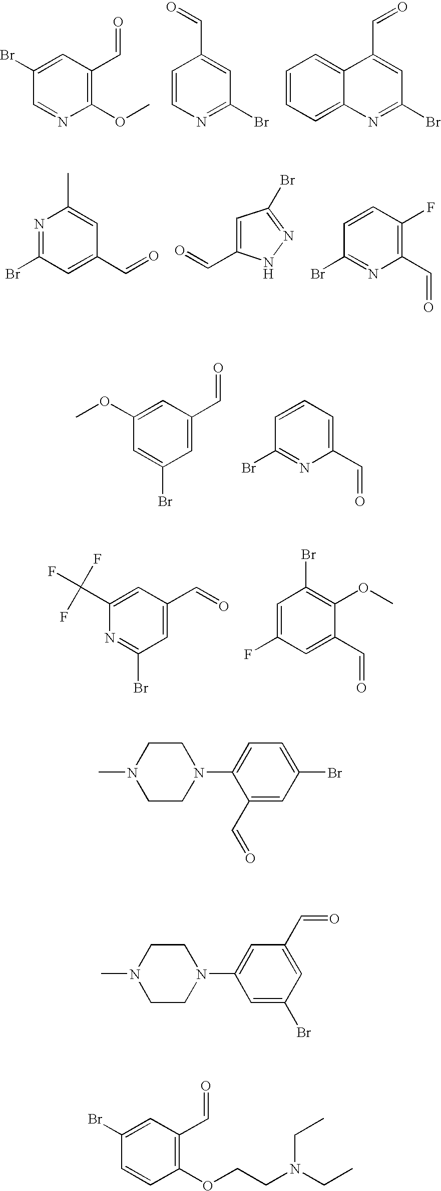

### Example 111

### Synthesis of 2,6-Disubstituted Perazines—General Procedure

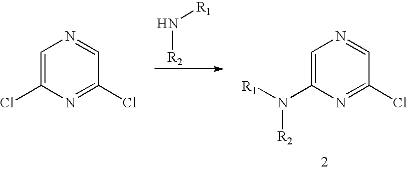

2,6-dichloropyrazine may be converted to a compound of formula 2 using amination techniques known to those skilled in the art. The reaction can be carried out via an adaptation of procedures found in the literature (Wolfe, J. P.; Buchwald, S. L. J. Org. Chem. 200, 65, 1144) wherein 2,6-dichloropyrazine is treated with an amine, a palladium (0) source and a base, optionally in suitable solvent, at temperature ranging from ambient temperature to 200° C. Suitable sources of palladium (0) include but are not limited to palladium (II) acetate and tri(dibenzylideneacetone) dipalladium (0). Typical bases for use in the reaction include, for example sodium tert-butoxide and cesium carbonate. The reaction can be carried out in neat amine or in suitable solvent. Toluene is an example of suitable solvent.

The following are representative examples of commercially available amines.

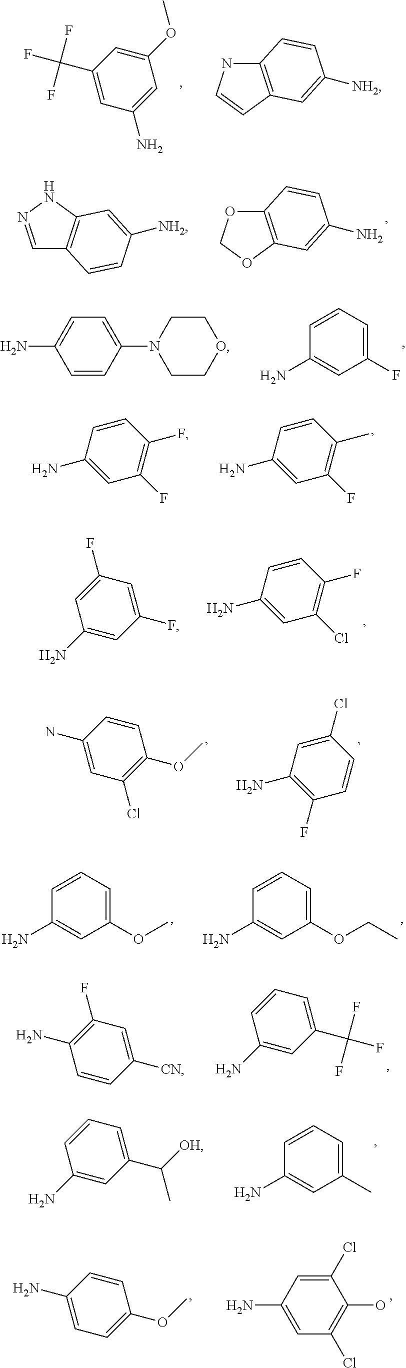

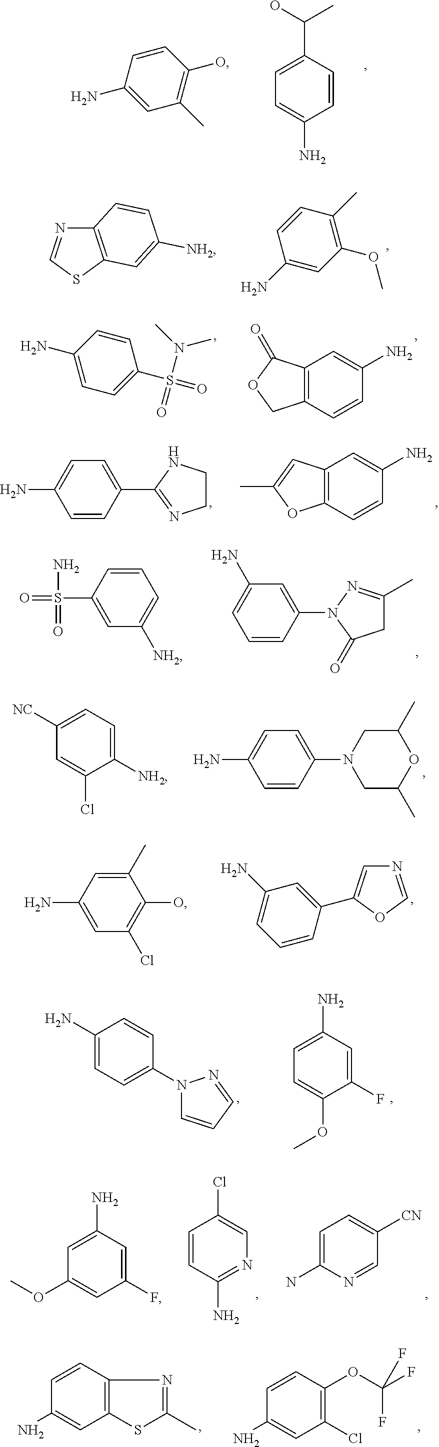

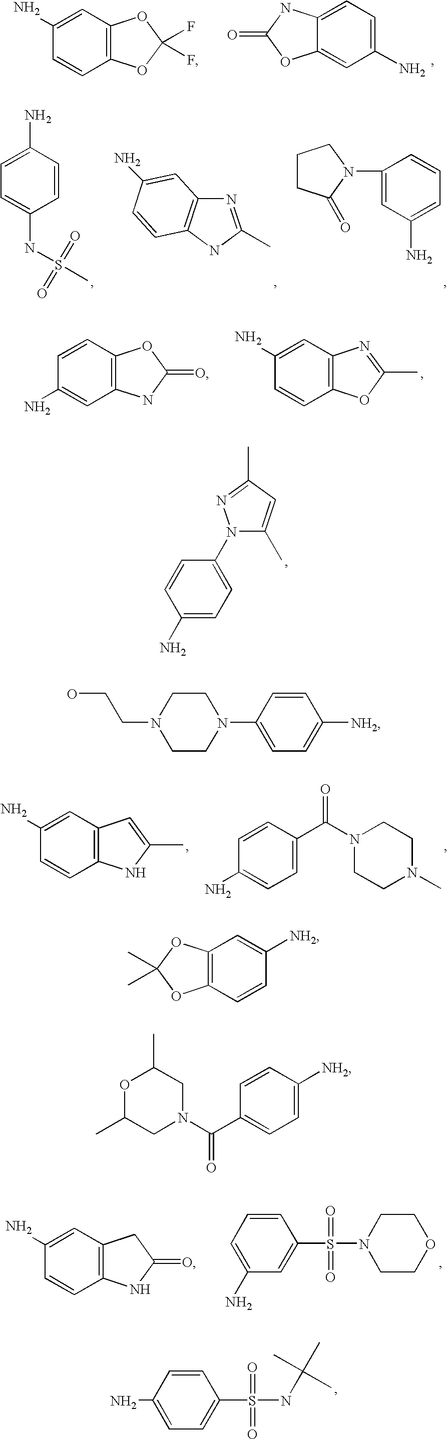

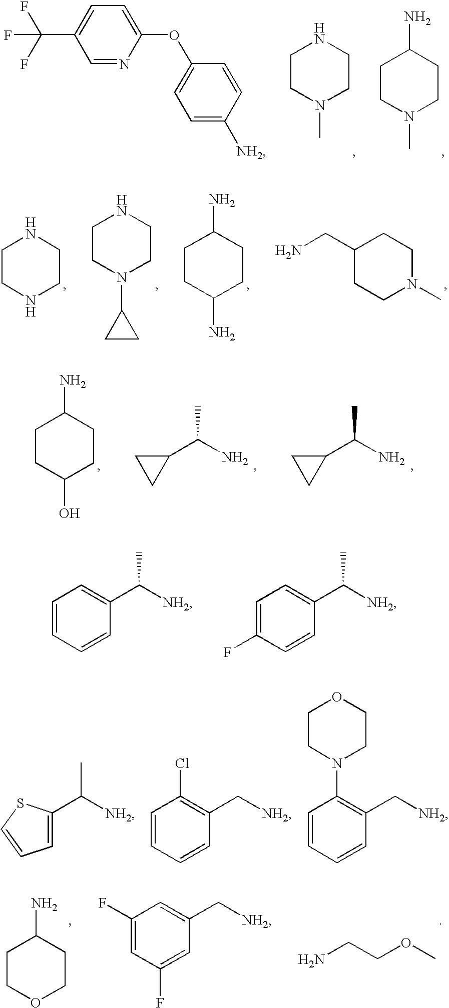

### Example 112

### Synthesis of 2,6-Disubstituted Perazines—General Procedure

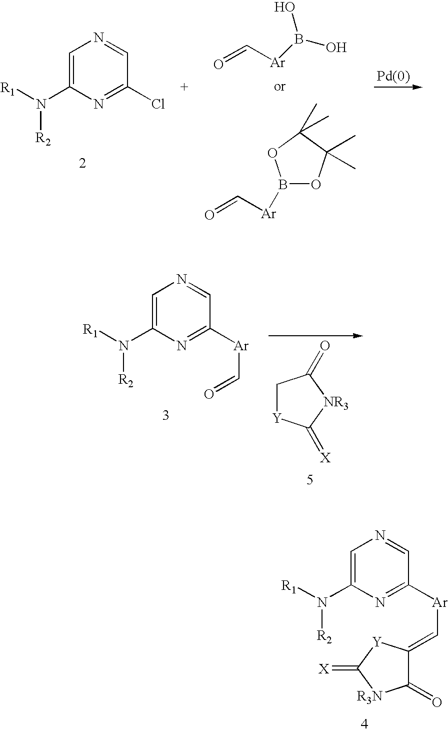

Compound 4 can be prepared from the treatment of the compound of formula 2 with boronic acid or boronic ester under Suzuki type conditions to give intermediate 3 (above). Intermediate 3 can then undergo a Knoevenagel type condensation with reagent 5 to give product 4.

### Example 113

### Synthesis of (S)-6-chloro-N-(1-cyclopropylethyl)pyrazin-2-amine

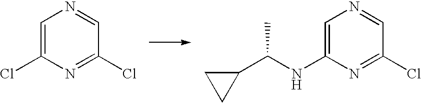

To 2,6-dichloropyrazine (200 mg, 1.361 mmol) in DMF was added (S)-1-cyclopropylethanamine (138 mL, 1.497 mmol) and DIEA (237 mL, 1.361 mmol). The reaction mixture was stirred at 70° C. overnight. Water was added resulting in precipitate formation. The resulting solid was isolated by filtration and allowed to air dry, yielding 110 mg (41% yield) (S)-6-chloro-N-(1-cyclopropylethyl)pyrazin-2-amine. LCMS (M+1=198).

### Example 114

### Synthesis of (S)-4-(6-(1-cyclopropylethylamino)pyrazin-2-yl)thiophene-2-carbaldehyde

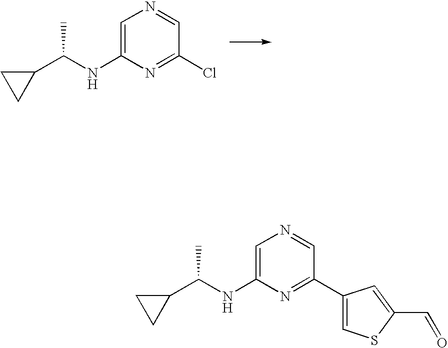

To (S)-6-chloro-N-(1-cyclopropylethyl)pyrazin-2-amine (50 mg, 0.254 mmol) in Dioxane/water (9:1) was added Cs₂CO₃ (248 mg, 0.761 mmol) and 5-formylthiophen-3-ylboronic acid (59 mg, 0.381 mmol). The mixture was degassed under N₂ for 5 minutes, then PdCl₂dppf (9 mg, 0.013 mmol) was added and the reaction microwaved at 110° C. for 80 minutes. The mixture was diluted with water and DCM, and product extracted into the organic layer. The organic layer was then prepared by TLC (1% MeOH/DCM) to yield 33 mg (48% yield) (S)-4-(6-(1-cyclopropylethylamino)pyrazin-2-yl)thiophene-2-carbaldehyde. LCMS (M+1=274).

### Example 115

### Synthesis of (S,Z)-5-((4-(6-(1-cyclopropylethylamino)pyrazin-2-yl)thiophen-2-yl)methylene)thiazolidine-2,4-dione

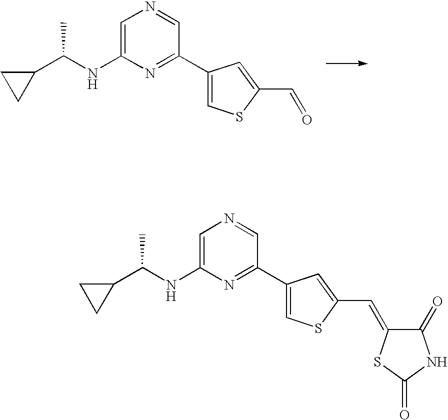

To (S)-4-(6-(1-cyclopropylethylamino)pyrazin-2-yl)thiophene-2-carbaldehyde (33 mg, 0.121 mmol) in EtOH was added thiazolidine-2,4-dione (42 mg, 0.363 mmol) and piperidine (36 mL, 0.363 mmol). The reaction was stirred at 80° C. for 2.75 hours. The solvent was then removed under reduced pressure, and the reaction was prepared by TLC (1% MeOH/DCM) to yield 23 mg (51% yield) (S,Z)-5-((4-(6-(1-cyclopropylethylamino)pyrazin-2-yl)thiophen-2-yl)methylene)thiazolidine-2,4-dione. LCMS (M+1=373).

### Example 116

### Synthesis of (S,Z)-5-(3-(6-(1-cyclopropylethylamino)pyrazin-2-yl)benzylidene)thiazolidine-2,4-dione

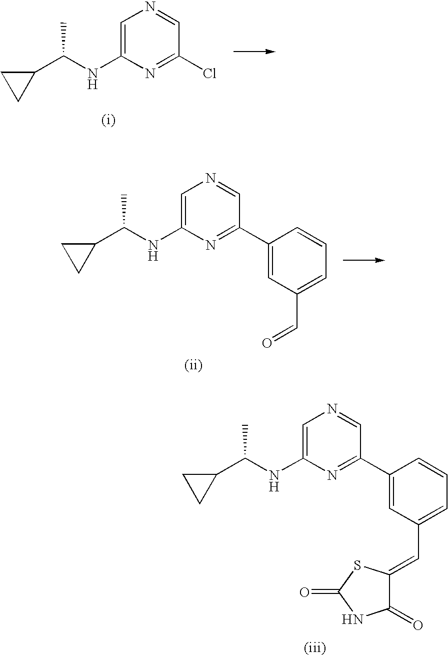

To (S)-6-chloro-N-(1-cyclopropylethyl)pyrazin-2-amine (47 mg, 0.239 mmol) in Dioxane/water (9:1) was added Cs₂CO₃ (233 mg, 0.716 mmol) and 3-formylphenylboronic acid (54 mg, 0.358 mmol). The mixture was degassed under N₂ for 5 minutes, then PdCl₂dppf (9 mg, 0.012 mmol) was added and the reaction microwaved at 120° C. for 30 minutes. The mixture was diluted with water and DCM, and product extracted into the organic layer. The organic layer was then dried over MgSO₄ and solvent removed under reduced pressure. The crude product (ii) was then dissolved in EtOH, and to it was added thiazolidine-2,4-dione (84 mg, 0.717 mmol) and piperidine (71 mL, 0.717 mmol). The reaction was stirred at 70° C. overnight. Solvent was removed under reduced pressure and purified by TLC (2% MeOH/DCM) to yield 11 mg (13% yield) (S,Z)-5-(3-(6-(1-cyclopropylethylamino)pyrazin-2-yl)benzylidene)thiazolidine-2,4-dione. LCMS (M+1=367).

### Example 117

### Synthesis of 6-chloro-N-(2-chlorobenzyl)pyrazin-2-amine

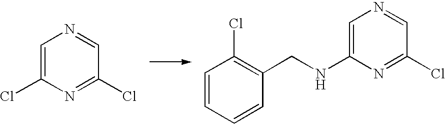

To 2,6-dichloropyrazine (500 mg, 3.401 mmol) in ACN was added (2-chlorophenyl)methanamine (450 mL, 3.741 mmol) and DIEA (593 mL, 3.401 mmol). The reaction was stirred at 70° C. overnight. The reaction mixture was prepared by TLC (1% MeOH/DCM) to yield 400 mg (46% yield) 6-chloro-N-(2-chlorobenzyl)pyrazin-2-amine. LCMS (M+1=254).

### Example 118

### Synthesis of (Z)-5-(3-(6-(2-chlorobenzylamino)pyrazin-2-yl)benzylidene)thiazolidine-2,4-dione

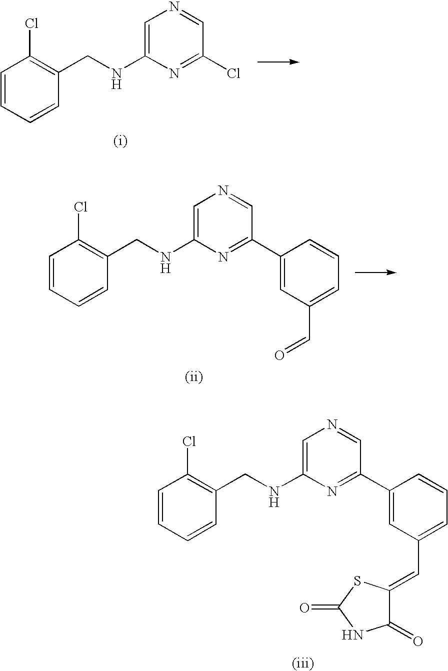

To 6-chloro-N-(2-chlorobenzyl)pyrazin-2-amine (200 mg, 0.791 mmol) in Dioxane/water (9:1) was added Cs₂CO₃ (773 mg, 2.372 mmol) and 3-formylphenylboronic acid (131 mg, 0.870 mmol) The mixture was degassed under N₂ for 5 minutes, then PdCl₂dppf (29 mg, 0.040 mmol) was added and the reaction microwaved at 120° C. for 20 minutes. The mixture was diluted with water and DCM, and product extracted into the organic layer. The organic layer was then dried over MgSO₄ and solvent removed under reduced pressure. The crude product (ii) was then dissolved in EtOH, and to it was added thiazolidine-2,4-dione (277 mg, 2.373 mmol) and piperidine (234 mL, 2.373 mmol). The reaction was stirred at 70° C. overnight. Solvent was removed under reduced pressure and purified by TLC (1% MeOH/DCM) to yield 80 mg (24% yield) (Z)-5-(3-(6-(2-chlorobenzylamino)pyrazin-2-yl)benzylidene)thiazolidine-2,4-dione. LCMS (M+1=423).

### Example 119

### Synthesis of (Z)-5-(4-(6-(2-chlorobenzylamino)pyrazin-2-yl)thiophen-2-yl)methylene)thiazolidine-2,4-dione

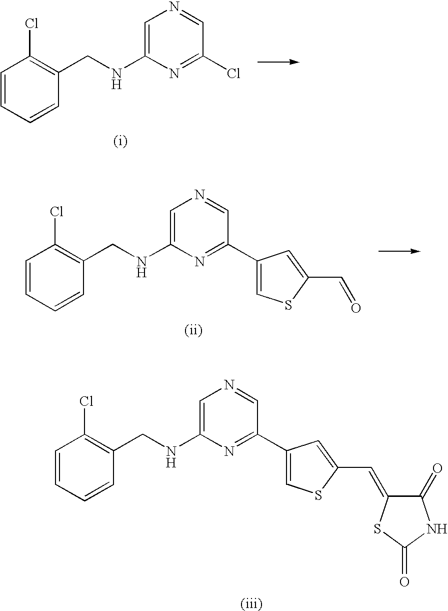

To 6-chloro-N-(2-chlorobenzyl)pyrazin-2-amine (200 mg, 0.791 mmol) in Dioxane/water (9:1) was added Cs2CO3 (773 mg, 2.372 mmol) and 5-formylthiophen-3-ylboronic acid (172 mg, 1.266 mmol). The mixture was degassed under N2 for 5 minutes, then PdCl2dppf (29 mg, 0.040 mmol) was added and the reaction microwaved at 120° C. for 40 minutes. The mixture was diluted with water and DCM, and product extracted into the organic layer. The organic layer was then dried over MgSO4 and solvent removed under reduced pressure. The crude product (ii) was then dissolved in EtOH, and to it was added thiazolidine-2,4-dione (277 mg, 2.373 mmol) and piperidine (234 mL, 2.373 mmol). The reaction was stirred at 70° C. overnight. Solvent was removed under reduced pressure and purified by TLC (5% MeOH/DCM) to yield (Z)-5-(4-(6-(2-chlorobenzylamino)pyrazin-2-yl)thiophen-2-yl)methylene)thiazolidine-2,4-dione. LCMS (M+1=429).

### Example 120

### Synthesis of 3-(6-((1r,4r)-4-hydroxycyclohexylamino)pyrazin-2-yl)benzaldehyde

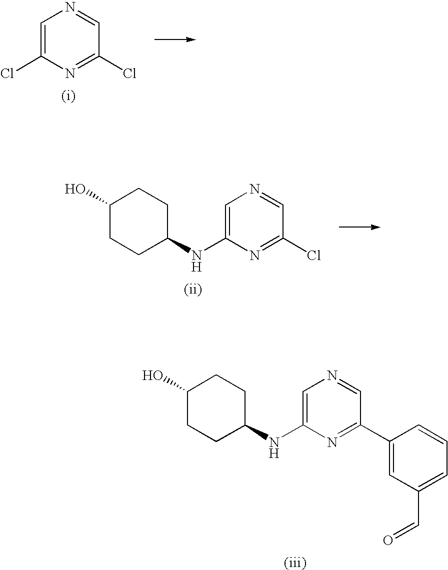

To 2,6-dichloropyrazine (400 mg, 2.721 mmol) in DMF was added (1r,4r)-4-aminocyclohexanol (344 mg, 2.993 mmol) and DIEA (949 mL, 5.442 mmol). The reaction was then stirred at 120° C. for 4 hours. The mixture was diluted with water and DCM, and product extracted into the organic layer. The organic layer was then dried over MgSO₄ and solvent removed under reduced pressure. The crude was prepared by TLC (1% MeOH/DCM) to yield (1r,4r)-4-(6-chloropyrazin-2-ylamino)cyclohexanol (ii). LCMS (M+1=228). Half of the product was then dissolved in Dioxane/water (9:1), then was added Cs₂CO₃ (1331 mg, 4.082 mmol) and 3-formylphenylboronic acid (306 mg, 2.041 mmol). The mixture was degassed under N₂ for 5 minutes, then PdCl₂dppf (50 mg, 0.068 mmol) was added and the reaction microwaved at 120° C. for 20 minutes. The mixture was diluted with water and DCM, and product extracted into the organic layer. The organic layer was then dried over MgSO₄ and prepared by TLC (2% MeOH/DCM) to yield 100 mg 3-(6-((1r,4r)-4-hydroxycyclohexylamino)pyrazin-2-yl)benzaldehyde. LCMS (M+1=298).

### Example 121

### Synthesis of 2-fluoro-5-(6-((1r,4r)-4-hydroxycyclohexylamino)pyrazin-2-yl)benzaldehyde

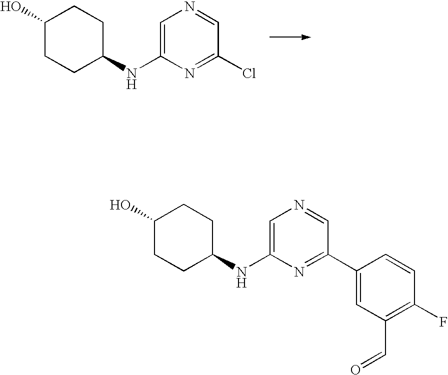

Half of product (ii) from procedure h. was then dissolved in Dioxane/water (9:1), then was added Cs₂CO₃ (1331 mg, 4.082 mmol) and 4-fluoro-3-formylphenylboronic acid (341 mg, 2.041 mmol) The mixture was degassed under N₂ for 5 minutes, then PdCl₂dppf (50 mg, 0.068 mmol) was added and the reaction microwaved at 120° C. for 20 minutes. The mixture was diluted with water and DCM, and product extracted into the organic layer. The organic layer was then dried over MgSO₄ and prepared by TLC (2% MeOH/DCM) to yield 270 mg 2-fluoro-5-(6-(1r,4r)-4-hydroxycyclohexylamino)pyrazin-2-yl)benzaldehyde. LCMS (M+1=316).

### Example 122

### Synthesis of (Z)-5-(3-(6-((1r,4r)-4-hydroxycyclohexylamino)pyrazin-2-yl)benzylidene)thiazolidine-2,4-dione

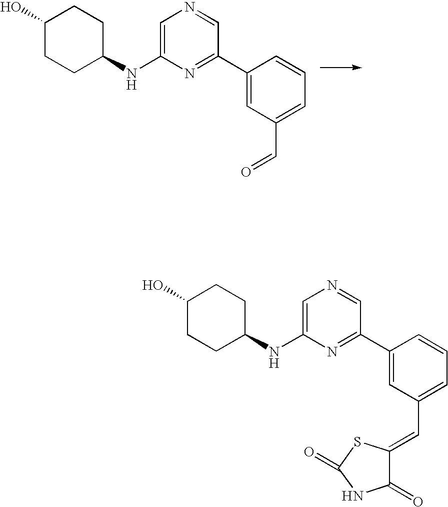

To 3-(6-((1r,4r)-4-hydroxycyclohexylamino)pyrazin-2-yl)benzaldehyde (100 mg, 0.337 mmol) in EtOH was added thiazolidine-2,4-dione (118 mg, 1.010 mmol) and piperidine (100 mL, 1.010 mmol). The reaction mixture was stirred at 80° C. over the weekend. Solvent was then removed under reduced pressure, and the mixture was then diluted in MeOH/DCM. Precipitate was collected by filtration, yielding (Z)-5-(3-(6-((1r,4r)-4-hydroxycyclohexylamino)pyrazin-2-yl)benzylidene)thiazolidine-2,4-dione. LCMS (M+1=397).

### Example 123

### Synthesis of (Z)-5-(2-fluoro-5-(6-(1r,4r)-4-hydroxycyclohexylamino)pyrazin-2-yl)benzylidene)thiazolidine-2,4-dione

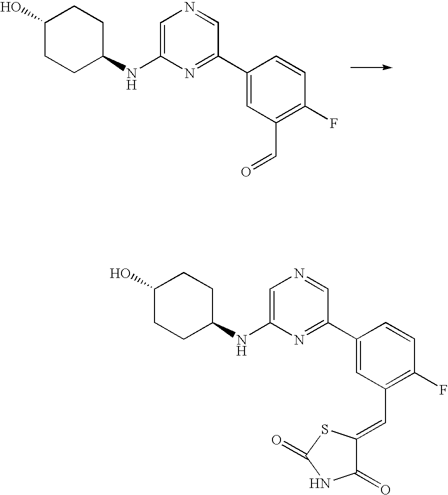

To 2-fluoro-5-(6-(1r,4r)-4-hydroxycyclohexylamino)pyrazin-2-yl)benzaldehyde (270 mg, 0.857 mmol) in EtOH was added thiazolidine-2,4-dione (301 mg, 2.571 mmol) and piperidine (254 mL, 2.571 mmol). The reaction mixture was stirred at 80° C. over the weekend. Solvent was then removed under reduced pressure, and mixture was then diluted in MeOH/DCM. Precipitate was collected by filtration, yielding (Z)-5-(2-fluoro-5-(6-((1r,4r)-4-hydroxycyclohexylamino)pyrazin-2-yl)benzylidene)thiazolidine-2,4-dione. LCMS (M+1=415).

### Example 124

### Synthesis of 2-fluoro-3-(6-(1-(4-fluorophenyl)ethyl amino)pyrazin-2-yl)benzaldehyde

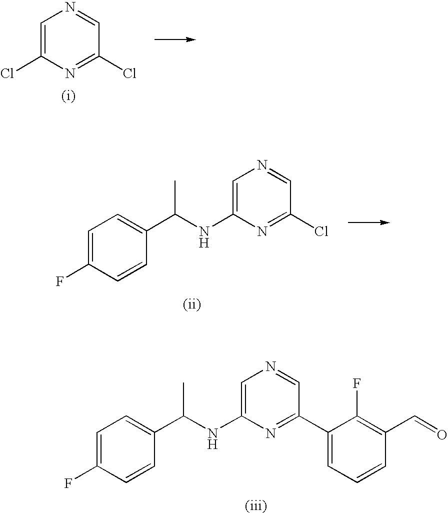

To 2,6-dichloropyrazine (i) (0.5 g, 32.5 mmol) in DMF was added diisopropylmethylamine (7.5 mL, 81.2 mmol) and 1-(4-fluorophenyl)ethanamine (0.45 mL, 3.40 mmol). The mixture was heated to 80° C. for overnight. The mixture was then cooled to room temperature, added water and product extracted in ethyl acetate. The organic layer was then concentrated and dried under reduced pressure to yield the crude oily product 6-chloro-N-(1-(4-fluorophenyl)ethyl)pyrazin-2-amine (ii) LCMS (M+1=252). To half of the product (ii) in 1.0 ml 1,4 dioxane/water (15%) was added 2-fluoro-3-formylphenylboronic acid (249.8 mg, 1.48 mmol) and cesium carbonate (970 mg, 2.97 mmol). The mixture was degassed under nitrogen for 10 minutes, PdCl₂dppf (40 mg, 0.05 mmol) was then added. The mixture was heated in the microwave at 120° C. for 20 minutes. After cooling to room temperature, added water and product extracted in dichloromethane. The organic layer was then concentrated and the product was purified using preparative TLC (2% MeOH/DCM) to yield 2-fluoro-3-(6-(1-(4-fluorophenyl)ethyl amino) pyrazin-2-yl)benzaldehyde (123 mg) LCMS (M+1=340).

### Example 125

### Synthesis of 5-(2-fluoro-3-(6-(1-(4-fluorophenyl)ethyl amino)pyrazin-2-yl)benzylidene)thiazolidine-2,4-dione

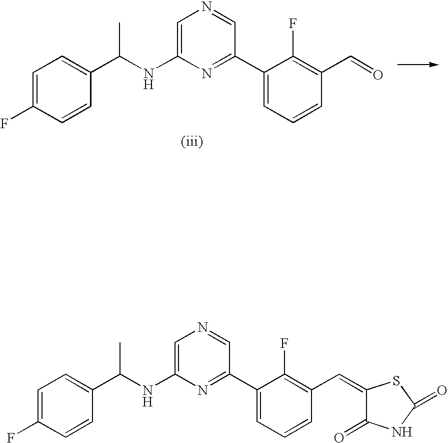

To 2-fluoro-3-(6-(1-(4-fluorophenyl)ethyl amino) pyrazin-2-yl)benzaldehyde (75 mg, 0.220 mmol) in 1.0 ml ethanol was added 2,4-thiazolidinedione (51.6 mg, 0.440 mmol) and piperidine (43.4 ul, 0.440 mmol). The mixture was stirred at 80° C. for overnight. After cooling, the reaction mixture was concentrated and diluted with dichloromethane and product purified using preparative TLC (2% MeOH/DCM) to yield 5-(2-fluoro-3-(6-(1-(4-fluorophenyl)ethyl amino) pyrazin-2-yl)benzylidene) thiazolidine-2,4-dione (10.0 mg). LCMS (M+1=439).

### Example 126

### Synthesis of 3-(6-(1-(4-fluorophenyl)ethyl amino)pyrazin-2-yl)benzaldehyde

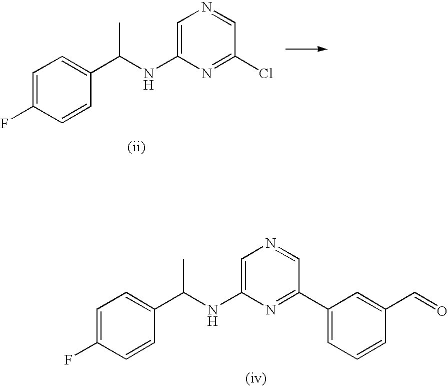

To half of the product (ii) in 1.0 ml 1,4 dioxane/water (15%) was 3-formylphenylboronic acid (240.2 mg, 1.40 mmol) and cesium carbonate (970 mg, 2.97 mmol). The mixture was degassed under nitrogen for 10 minutes, PdCl₂dppf (40 mg, 0.05 mmol) was then added. The mixture was heated in the microwave at 120° C. for 20 minutes. After cooling to room temperature, added water and product extracted in dichloromethane. The organic layer was then concentrated and the product was purified using preparative TLC (2% MeOH/DCM) to yield 3-(6-(1-(4-fluorophenyl)ethyl amino) pyrazin-2-yl)benzaldehyde (134 mg). LCMS (M+1=322).

### Example 127

### Synthesis of 5-(3-(6-(1-(4-fluorophenyl)ethyl amino)pyrazin-2-yl)benzylidene)thiazolidine-2,4-dione

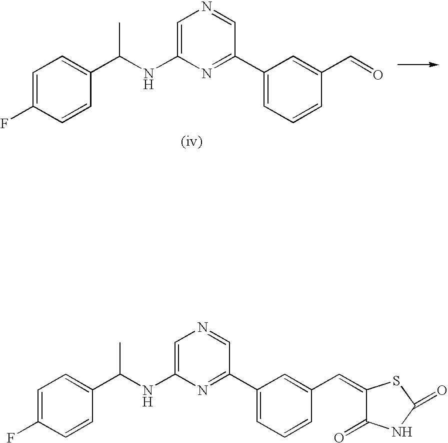

To 3-(6-(1-(4-fluorophenyl)ethyl amino) pyrazin-2-yl)benzaldehyde (75 mg, 0.220 mmol) in 1.0 ml ethanol was added 2,4-thiazolidinedione (51.6 mg, 0.440 mmol) and piperidine (43.4 ul, 0.440 mmol). The mixture was stirred at 80° C. for overnight. After cooling, the reaction mixture was concentrated and diluted with dichloromethane and product purified using preparative TLC (2% MeOH/DCM) to yield (E)-5-(3-(6-(1-(4-fluorophenyl)ethyl amino) pyrazin-2-yl)benzylidene) thiazolidine-2,4-dione (12.0 mg). LCMS (M+1=421).

### Example 128

### Synthesis of (S)-3-(6-(1-phenylethylamino)pyrazin-2-yl)benzaldehyde

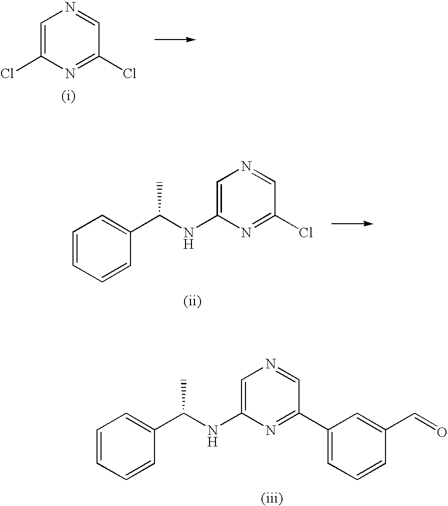

To 2,6-dichloropyrazine (i) (0.6 g, 4.08 mmol) in DMF was added diisopropylmethylamine (0.85 mL, 4.89 mmol) and (S)-1-phenylethanamine (0.52 mL, 4.08 mmol). The mixture was heated to 80° C. for overnight. The mixture was then cooled to room temperature, added water and product extracted in ethyl acetate. The organic layer was then concentrated and dried under reduced pressure to yield the crude oily product (S)-6-chloro-N-(1-phenylethyl)pyrazin-2-amine (ii) (726 mg) LCMS (M+1=234). To the product (ii) (250 mg, 1.068 mmol) in 1.0 ml DMF/water (15%) was added 3-formylphenylboronic acid (240.2 mg, 1.602 mmol) and cesium carbonate (1000 mg, 3.19 mmol). The mixture was degassed under nitrogen for 10 minutes, PdCl₂dppf (44 mg, 0.05 mmol) was then added. The mixture was heated in the microwave at 120° C. for 20 minutes. After cooling to room temperature, added water and the product extracted in dichloromethane. The organic layer was then concentrated and the product was purified using preparative TLC (2% MeOH/DCM) to yield (iii) (S)-3-(6-(1-phenylethylamino) pyrazin-2-yl)benzaldehyde (123 mg) LCMS (M+1=304).

### Example 129

### Synthesis of (S)-5-(3-(6-(1-phenylethylamino)pyrazin-2-yl)benzylidene)thiazolidine-2,4-dione

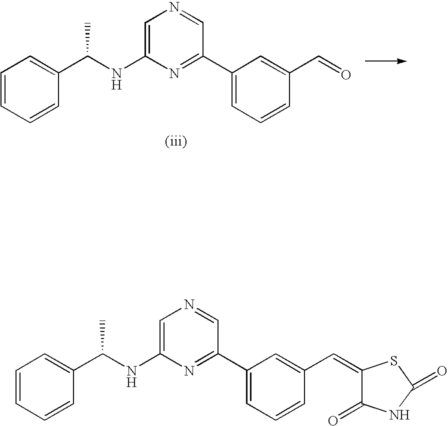

To (S)-3-(6-(1-phenylethylamino)pyrazin-2-yl)benzaldehyde (75 mg, 0.246 mmol) in 1.0 ml ethanol was added 2,4-thiazolidinedione (57.6 mg, 0.492 mmol) and piperidine (41.8 ul, 0.492 mmol). The mixture was stirred at 80° C. for overnight. After cooling, the reaction mixture was concentrated and diluted with dichloromethane and product purified using preparative TLC (2% MeOH/DCM) to yield (S)-5-(3-(6-(1-phenylethylamino) pyrazin-2-yl)benzylidene) thiazolidine-2,4-dione (12.0 mg). LCMS (M+1=421).

### Example 130

### Synthesis of (S)-2-fluoro-3-(6-(1-phenylethylamino)pyrazin-2-yl)benzaldehyde

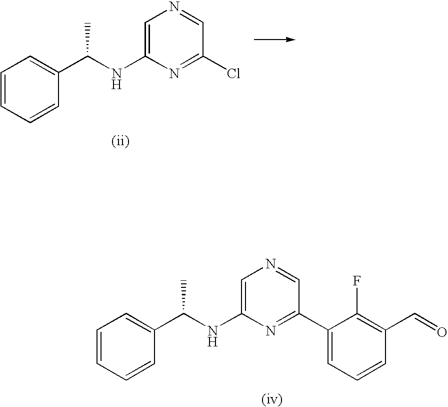

To (S)-6-chloro-N-(1-phenylethyl)pyrazin-2-amine (ii) (250 mg, 1.068 mmol) in 1.0 ml 1,4 DMF/water (15%) was added 2-fluoro-3-formylphenylboronic acid (269.8 mg, 1.602 mmol) and cesium carbonate (970 mg, 2.97 mmol). The mixture was degassed under nitrogen for 10 minutes, PdCl₂dppf (40 mg, 0.05 mmol) was then added. The mixture was heated in the microwave at 120° C. for 20 minutes. After cooling to room temperature, added water and product extracted in dichloromethane. The organic layer was then concentrated and the product was purified using preparative TLC (2% MeOH/DCM) to yield (iv) (S)-2-fluoro-3-(6-(1-phenylethylamino) pyrazin-2-yl)benzaldehyde (110 mg) LCMS (M+1=322).

### Example 131

### Synthesis of (S)-5-(2-fluoro-3-(6-(1-phenylethylamino)pyrazin-2-yl)benzylidene)thiazolidine-2,4-dione

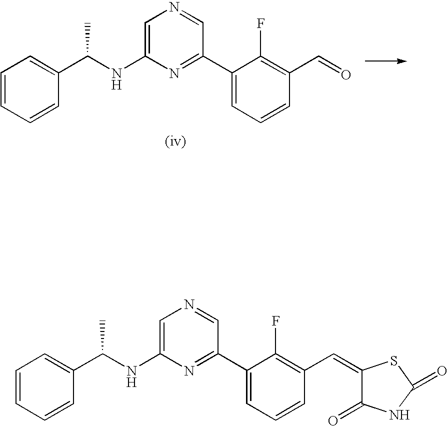

To) (S)-2-fluoro-3-(6-(1-phenylethylamino) pyrazin-2-yl)benzaldehyde (75 mg, 0.233 mmol) in 1.0 ml ethanol was added 2,4-thiazolidinedione (54.6 mg, 0.465 mmol) and piperidine (40.0 ul, 0.465 mmol). The mixture was stirred at 80° C. for overnight. After cooling, the reaction mixture was concentrated and diluted with dichloromethane and product purified using preparative TLC (2% MeOH/DCM) to yield (S)-5-(2-fluoro-3-(6-(1-phenylethylamino) pyrazin-2-yl)benzylidene) thiazolidine-2,4-dione (10.0 mg). LCMS (M+1=421).

### Example 132

### Synthesis of (S)-3-methyl-5-(6-(1-phenylethylamino)pyrazin-2-yl)benzaldehyde

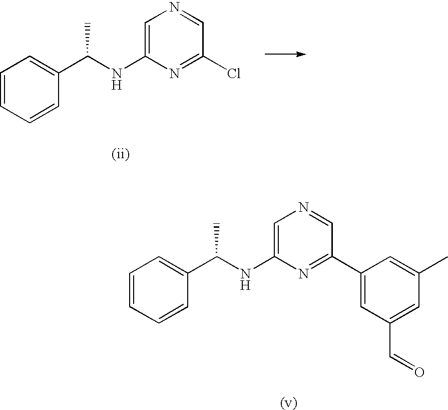

To (S)-6-chloro-N-(1-phenylethyl)pyrazin-2-amine (ii) (250 mg, 1.068 mmol) in 1.0 ml 1,4 DMF/water (15%) was added 3-formyl-5-methylphenylboronic acid (263.8 mg, 1.602 mmol) and cesium carbonate (970 mg, 2.97 mmol). The mixture was degassed under nitrogen for 10 minutes, PdCl₂dppf (40 mg, 0.05 mmol) was then added. The mixture was heated in the microwave at 120° C. for 20 minutes. After cooling to room temperature, added water and product extracted in dichloromethane. The organic layer was then concentrated and the product was purified using preparative TLC (2% MeOH/DCM) to yield (v) (S)-3-methyl-5-(6-(1-phenylethylamino) pyrazin-2-yl)benzaldehyde (125 mg) LCMS (M+1=318).

### Example 133

### Synthesis of (S)-5-(3-methyl-5-(6-(1-phenylethylamino)pyrazin-2-yl)benzylidene)thiazolidine-2,4-dione

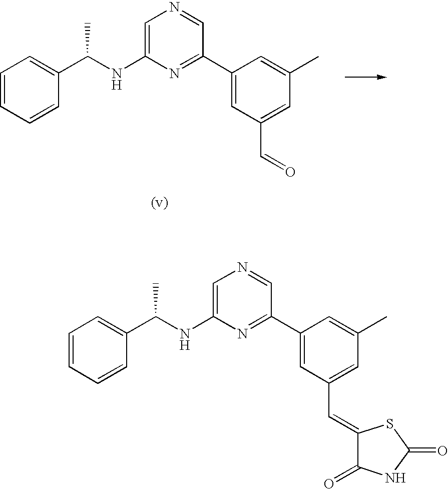

To) (S)-3-methyl-5-(6-(1-phenylethylamino)pyrazin-2-yl)benzaldehyde (75 mg, 0.235 mmol) in 1.0 ml ethanol was added 2,4-thiazolidinedione (55.2 mg, 0.471 mmol) and piperidine (40.0 ul, 0.471 mmol). The mixture was stirred at 80° C. for overnight. After cooling, the reaction mixture was concentrated and diluted with dichloromethane and product purified using preparative TLC (2% MeOH/DCM) to yield (S)-5-(3-methyl-5-(6-(1-phenylethylamino) pyrazin-2-yl)benzylidene) thiazolidine-2,4-dione (11.0 mg). LCMS (M+1=417).

### Example 134

### Synthesis of 3-(6-(4-aminocyclohexylamino)pyrazin-2-yl)benzaldehyde

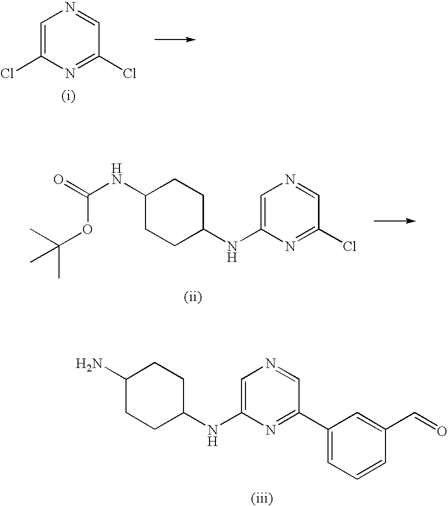

To 2,6-dichloropyrazine (i) (0.6 g, 4.08 mmol) in DMF was added diisopropylmethylamine (7.5 mL, 81.2 mmol) and tert-butyl (1r,4r)-4-aminocyclohexylcarbamate (874 mg, 4.08 mmol). The mixture was heated to 80° C. for overnight. The mixture was then cooled to room temperature, added water. Filtered the white solid to yield 6-chloro-N-(1-(4-fluorophenyl)ethyl)pyrazin-2-amine (ii) LCMS (M+1=327). To (ii) (250 mg, 0.764 mmols) in 1.0 ml 1,4 DMF/water (15%) was added 3-formylphenylboronic acid (172.0 mg, 1.146 mmol) and cesium carbonate (747 mg, 2.29 mmol). The mixture was degassed under nitrogen for 10 minutes, PdCl₂dppf (31.2 mg, 0.04 mmol) was then added. The mixture was heated in the microwave at 120° C. for 20 minutes. After cooling to room temperature, added water and product extracted in dichloromethane. The organic layer was then concentrated and the product was purified using preparative TLC (2% MeOH/DCM) to yield tert-butyl 4-(6-(3-formylphenyl)pyrazin-2-ylamino) cyclohexylcarbamate (130 mg) LCMS (M+1=397). The purified product was dissolved in 1:1 mixture of dichloromethane and trifluoroacetic acid at room temperature for 1 hr. Concentrated the reaction mixture to yield the product 3-(6-(4-aminocyclohexylamino)pyrazin-2-yl)benzaldehyde. LCMS (M+1=297).

### Example 135

### Synthesis of 5-(3-(6-(4-aminocyclohexylamino)pyrazin-2-yl)benzylidene)thiazolidine-2,4-dione

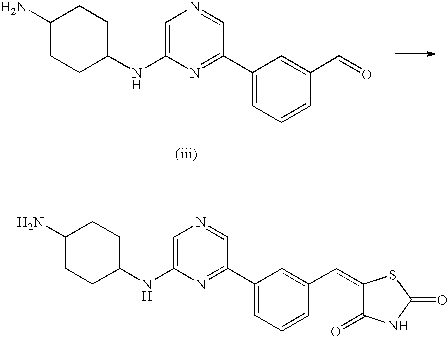

To 3-(6-(4-aminocyclohexylamino)pyrazin-2-yl)benzaldehyde (75 mg, 0.252 mmol) in 1.0 ml ethanol was added 2,4-thiazolidinedione (59.2 mg, 0.505 mmol) and piperidine (43.4 ul, 0.505 mmol). The mixture was stirred at 80° C. for overnight. After cooling, the reaction mixture was concentrated and diluted with dichloromethane and product purified using preparative TLC (2% MeOH/DCM) to yield 5-(3-(6-(4-aminocyclohexylamino) pyrazin-2-yl)benzylidene) thiazolidine-2,4-dione (9.0 mg). LCMS (M+1=396).

### Biodata Test Methods

### Example 136

### CK2 Assay Method

Modulatory activity of compounds described herein was assessed in vitro in cell-free CK2 assays by the following method.

Test compounds in aqueous solution were added at a volume of 10 microliters, to a reaction mixture comprising 10 microliters Assay Dilution Buffer (ADB; 20 mM MOPS, pH 7.2, 25 mM beta-glycerolphosphate, 5 mM EGTA, 1 mM sodium orthovanadate and 1 mM dithiothreitol), 10 microliters of substrate peptide (RRRDDDSDDD, dissolved in ADB at a concentration of 1 mM), 10 microliters of recombinant human CK2 (25 ng dissolved in ADB; Upstate). Reactions were initiated by the addition of 10 microliters of ATP Solution (90% 75 mM MgCl₂, 75 micromolar ATP dissolved in ADB; 10% [γ-³³P]ATP (stock 1 mCi/100 μl; 3000 Ci/mmol (Perkin Elmer) and maintained for 10 minutes at 30 degrees C. The reactions were quenched with 100 microliters of 0.75% phosphoric acid, then transferred to and filtered through a phosphocellulose filter plate (Millipore). After washing each well 5 times with 0.75% phosphoric acid, the plate was dried under vacuum for 5 min and, following the addition of 15 ul of scintillation fluid to each well, the residual radioactivity was measured using a Wallac luminescence counter.

### Example 137

### PIM-1 Assay Method

The following procedure was used to assay the PIM-1 kinase activity of compounds of the invention. Other methods for assaying PIM-1 and other PIM kinases, as well as methods to assay for activity against the various kinases are known in the art.

In a final reaction volume of 50 ul, recombinant PIM-1 (1 ng) was incubated with 12 mM MOPS pH 7.0, 0.4 mM EDTA, glycerol 1%, brij 35 0.002%, 2-mercaptoethanol 0.02%, BSA 0.2 mg/ml, 100 uM KKRNRTLTK, 10 mM MgAcetate, 15 uM ATP, [γ-³³P-ATP] (specific activity approx. 500 cpm/pmol), DMSO 4% and test inhibitor compound at the required concentration. The reaction was initiated by the addition of the Magnesium ATP mixture. After 40 min incubation at 23° C., the reactions were quenched by the addition of 100 ul 0.75% Phosphoric acid, and the labeled peptide collected by filtration through a phosphocellulose filter plate. The plate was washed 4 times with 0.075% phosphoric acid (100 ul per well) and then, after the addition of scintillation fluid (20 ul per well), the counts were measured by a scintillation counter.

### Example 138

### PIM-2 Assay Method

PIM-2 Assay Method: Test compounds dissolved and diluted in DMSO (2 μl) were added to a reaction mixture comprising 10 μl of 5× Reaction Buffer (40 mM MOPS pH 7.0, 5 mM EDTA), 10 μl of recombinant human PIM2 solution (4 ng PIM-2 dissolved in dilution buffer (20 mM MOPS pH 7.0; EDTA 1 mM; 5% Glycerol; 0.01% Brij 35; 0.1%; 0.1% 2-mercaptoethanol; 1 mg/ml BSA)) and 8 ul of water. Reactions were initiated by the addition of 10 ul of ATP Solution (49% (15 mM MgCl₂; 75 uM ATP) 1% ([γ-³³P]ATP: Stock 1 mCi/1000; 3000Ci/mmol (Perkin Elmer)) and 10 ul of substrate peptide solution (RSRSSYPAGT, dissolved in water at a concentration of 1 mM), Reactions were maintained for 10 min at 30° C. The reactions were quenched with 100 ul of 0.75% Phosphoric acid, then transferred to and filtered through a Phosphocellulose filter plate (Millipore, MSPH-N6B-50). After washing each well 4 times with 0.75% Phosphoric acid, scintillation fluid (20 uL) was added to each well and the residual radioactivity was measured using a Wallac luminescence counter.

### Example 139

### Cell Proliferation Modulatory Activity

A representative cell-proliferation assay protocol using Alamar Blue dye (stored at 4° C., use 20 ul per well) is described hereafter.

### 96-Well Plate Setup and Compound Treatment

a. Split and trypsinize cells.

b. Count cells using hemocytometer.

c. Plate 4,000-5,000 cells per well in 100 μl of medium and seed into a 96-well plate according to the following plate layout. Add cell culture medium only to wells B10 to B12. Wells B1 to B9 have cells but no compound added.

| | | | |
|---|---|---|---|
| 1 2 3 | 4 5 6 | 7 8 9 | 10 11 12 |

| | |
|---|---|
| A | EMPTY |

| B | NO COMPOUND ADDED | | | Medium | |
|---|---|---|---|---|---|
| | | | | Only | |
| C | 10 nM | 100 nM | 1 uM | 10 uM | Control |
| D | 10 nM | 100 nM | 1 uM | 10 uM | Comp1 |
| E | 10 nM | 100 nM | 1 uM | 10 uM | Comp2 |
| F | 10 nM | 100 nM | 1 uM | 10 uM | Comp3 |
| G | 10 nM | 100 nM | 1 uM | 10 uM | Comp4 |

| | |
|---|---|
| H | EMPTY |

d. Add 100 μl of 2× drug dilution to each well in a concentration shown in the plate layout above. At the same time, add 100 μl of media into the control wells (wells B10 to B12). Total volume is 200 μl/well.

e. Incubate four (4) days at 37° C., 5% CO₂ in a humidified incubator.

f. Add 20 μl Alamar Blue reagent to each well.

g. Incubate for four (4) hours at 37° C., 5% CO₂ in a humidified incubator.

h. Record fluorescence at an excitation wavelength of 544 nm and emission wavelength of 590 nm using a microplate reader.

In the assays, cells are cultured with a test compound for approximately four days, the dye is then added to the cells and fluorescence of non-reduced dye is detected after approximately four hours. Different types of cells can be utilized in the assays (e.g., HCT-116 human colorectal carcinoma cells, PC-3 human prostatic cancer cells and MiaPaca human pancreatic carcinoma cells).

Activity of representative compounds described herein in these in vitro and cellular assays are summarized in Tables 1-6. Additional compound examples are shown in Table 7.

TABLE 1

| | | | AB: | AB: | AB: | | | | |
|---|---|---|---|---|---|---|---|---|---|
| | AB: HCT-116 | AB: K-562 | MV-4-11 | MDAMB231 | MiaPaCa | AB: | CK2: | PIM1: | PIM2: |
| Structure | IC50 (μM) | IC50 (μM) | IC50 (μM) | IC50 (μM) | IC50 (μM) | PC3 (μM) | IC50 (μM) | IC50 (μM) | IC50 (μM) |
| 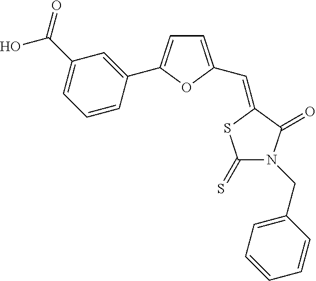 | >10 | | | | | | | | |
| 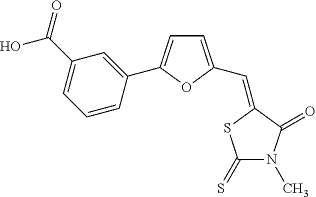 | >10 | | | | | | | | |
| 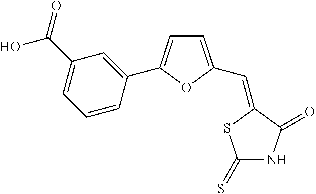 | >10 | >10 | >10 | | | | | | |
| 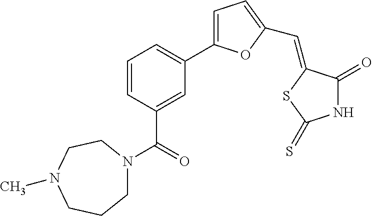 | | 2.891 | 0.406 | >10 | 2.295 | >30 | 0.047 | 0.005 | 0.013 |
| 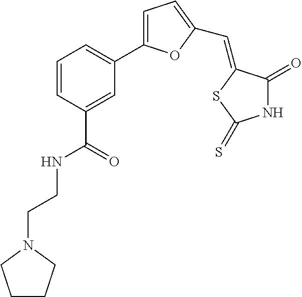 | | >10 | >10 | >10 | >10 | >30 | | 0.074 | 0.704 |
| 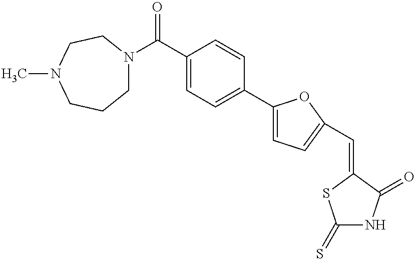 | | >10 | >10 | >10 | >10 | >30 | | 0.025 | |
| 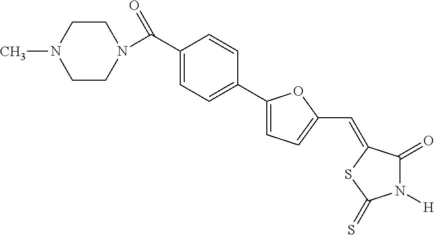 | | >10 | >10 | >10 | >10 | >30 | | 0.064 | |
| 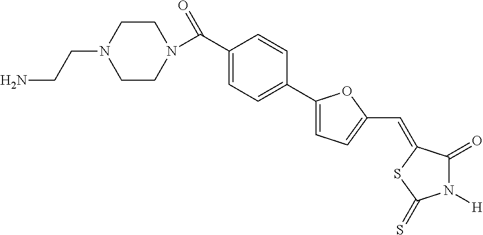 | | >10 | >1 | >10 | >10 | >30 | 0.196 | 0.019 | |
| 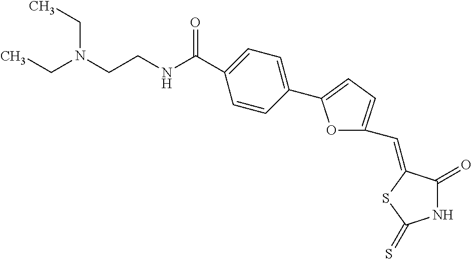 | | >10 | >10 | >10 | >10 | >30 | 0.131 | 0.006 | |
| 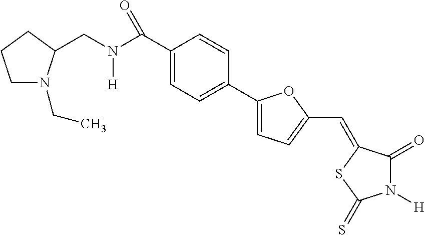 | | >10 | 0.21 | >10 | 3.948 | >30 | | 0.028 | |
| 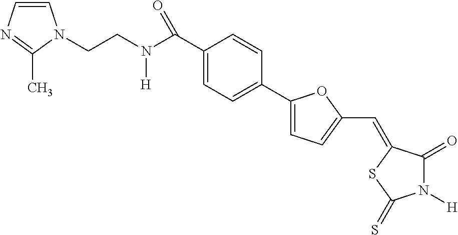 | | >10 | >10 | >10 | >10 | >30 | 0.226 | 0.014 | |
| 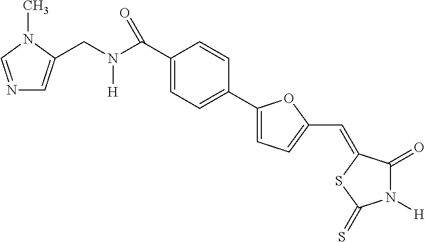 | | >10 | >10 | >10 | >10 | >30 | 0.108 | 0.035 | |
| 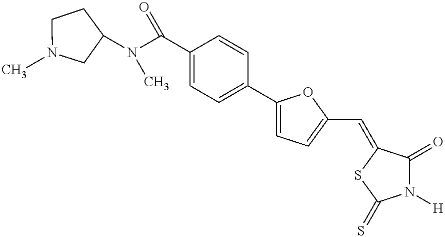 | | >10 | >10 | >10 | >10 | >30 | | 0.037 | |
| 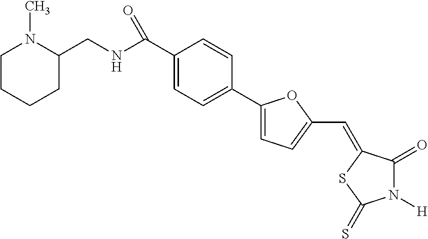 | | >10 | >10 | >10 | >10 | >30 | 0.471 | 0.037 | |
| 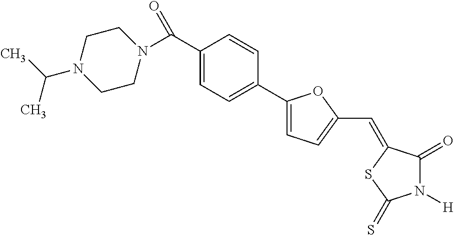 | | >10 | >10 | >10 | >10 | >30 | | 0.067 | |
| 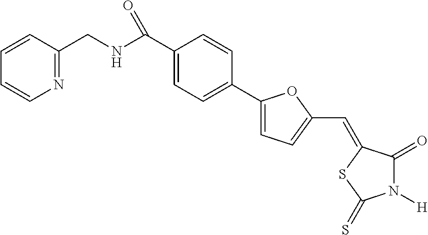 | | >10 | 8.83 | >10 | >10 | >30 | 0.04 | 0.286 | |
| 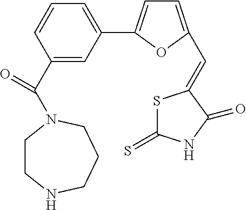 | | >10 | >10 | >10 | >10 | >30 | 0.412 | 0.009 | |
| 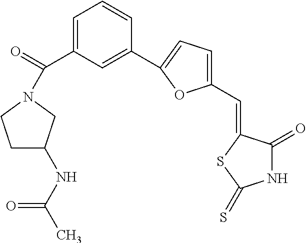 | | >10 | >10 | >10 | >10 | >30 | 0.444 | 0.038 | |
| 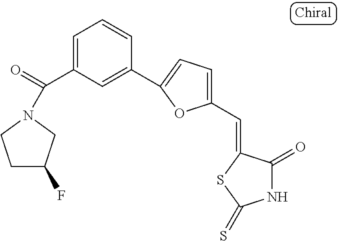 | | >10 | 9.5 | >10 | >10 | >30 | 0.012 | 0.013 | |
| 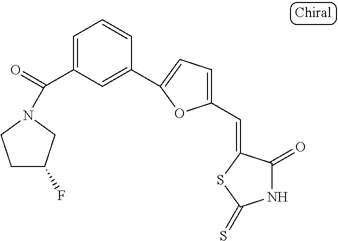 | | >10 | >10 | >10 | >10 | >30 | 0.026 | 0.04 | |
| 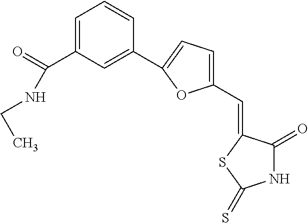 | | >10 | >10 | >10 | >10 | >30 | 0.264 | 0.028 | |
| 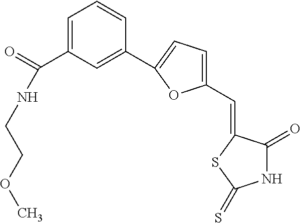 | | >10 | >10 | >10 | >10 | >30 | 0.123 | 0.031 | |
| 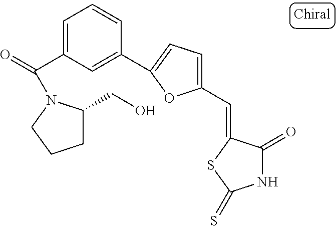 | | >10 | 0.175 | >10 | >10 | >30 | 0.037 | 0.012 | |
| 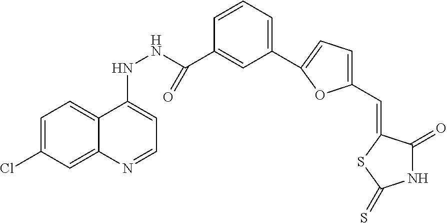 | | >10 | >10 | >10 | >10 | >30 | 0.266 | 0.146 | |
| 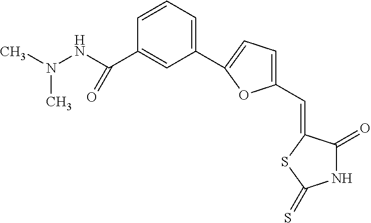 | | >10 | >10 | >10 | >10 | >30 | 0.094 | 0.036 | |
| 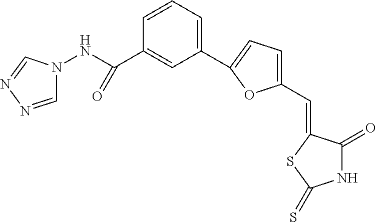 | | >10 | | >10 | >10 | >30 | 0.281 | 0.047 | |
| 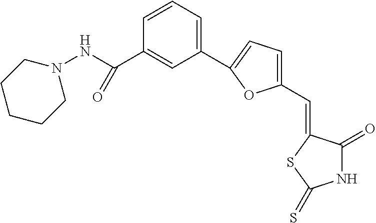 | | >10 | >10 | >10 | >10 | >30 | 0.551 | 0.086 | |
| 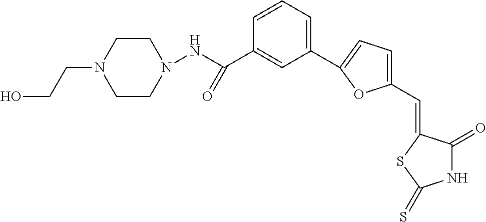 | | >10 | >10 | >10 | >10 | >30 | 0.288 | 0.162 | |
| 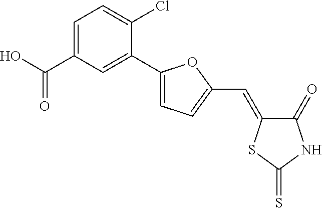 | | >10 | >10 | >10 | >10 | >30 | 0.027 | 0.199 | |
| 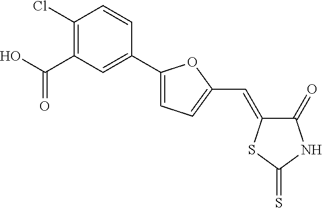 | | >10 | >10 | >10 | >10 | | 0.107 | 0.226 | |
| 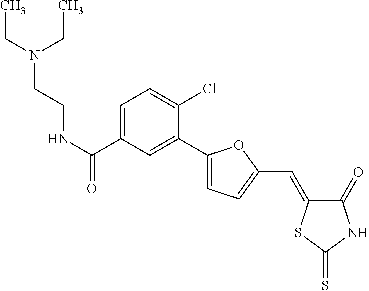 | | 0.064 | <3.e−002 | >10 | | >10 | 0.003 | <1.e−003 | |
| 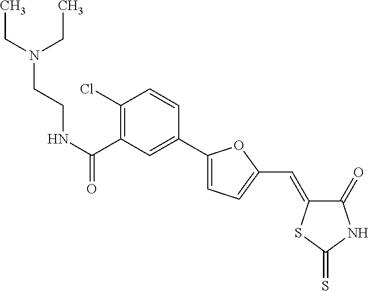 | | >10 | | >10 | >10 | >10 | 0.01 | 0.002 | |
| 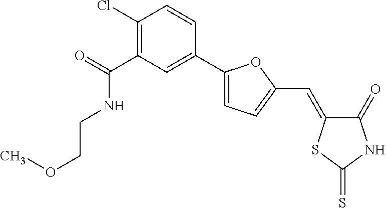 | | >10 | | >10 | >10 | >10 | 0.054 | 0.042 | |
| 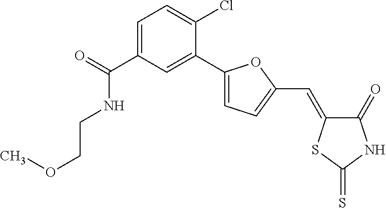 | | 7.617 | | >10 | >10 | >10 | 0.01 | 0.003 | |
| 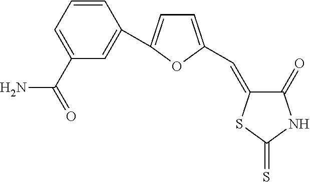 | | >10 | >10 | >10 | >10 | >10 | 0.004 | 0.004 | |
| 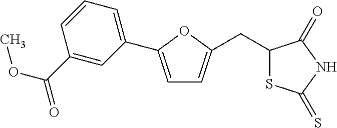 | | >10 | >10 | >10 | >10 | | 0.003 | 0.006 | |
| 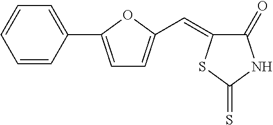 | | >10 | >10 | >10 | >10 | >10 | 0.004 | 0.059 | |
| 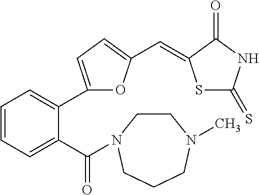 | | | | | | | >5 | 1.295 | |
| 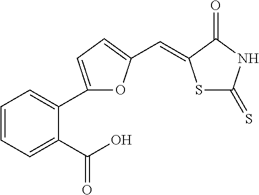 | | | | | | | 3.787 | 4.327 | |
| 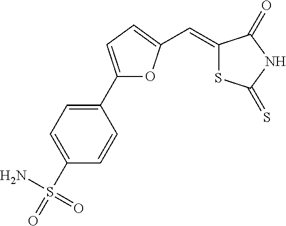 | | | | | | >10 | 0.009 | 0.015 | |

TABLE 2

| | | | | AB: | AB: | AB: | AB: |
|---|---|---|---|---|---|---|---|
| | CK2: | PIM1: | AB: | PC3 | MV-4-11 | MiaPaCa | K-562 |
| | IC50 | IC50 | MDAMB231 | IC50 | IC50 | IC50 | IC50 |
| Structure | (μM) | (μM) | IC50 (μM) | (μM) | (μM) | (μM) | (μM) |
| 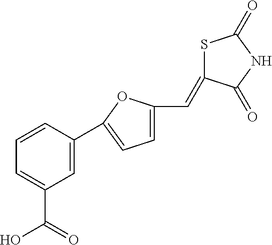 | 0.117 | >5 | | | | | |
| 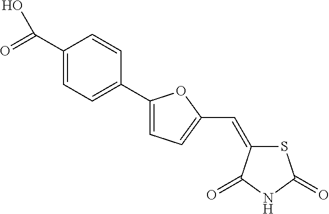 | 0.009 | 0.041 | >10 | >30 | >1 | >10 | >10 |
| 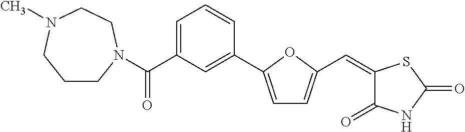 | 0.011 | 0.111 | >10 | >30 | >1 | >10 | >10 |
| 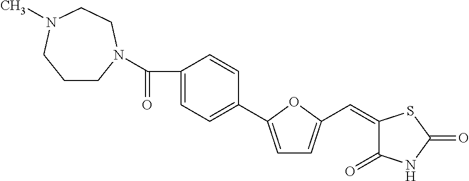 | 0.43 | 0.266 | >10 | >30 | >1 | >10 | >10 |
| 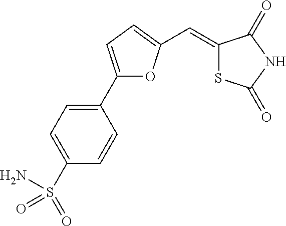 | 0.035 | 0.051 | | >10 | | | |

TABLE 3

| | CK2: IC50 | PIM1: IC50 |
|---|---|---|
| Structure | (μM) | (μM) |
| 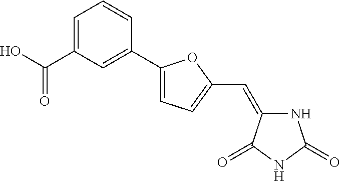 | 2.059 | >5 |
| 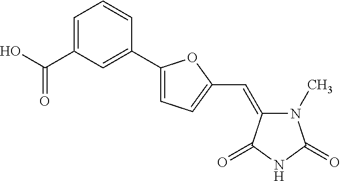 | 1.455 | >5 |
| 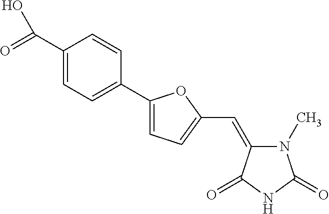 | 0.216 | 0.829 |
| 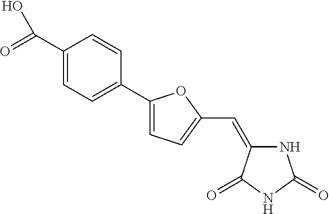 | 0.166 | 0.805 |
| 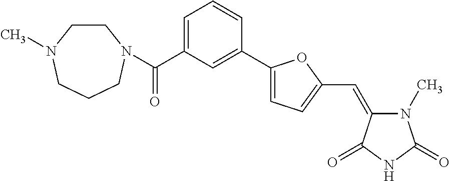 | 1.824 | >5 |
| 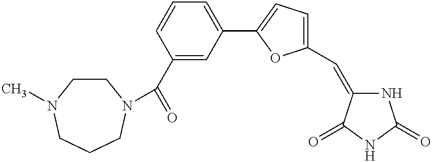 | >5 | >5 |
| 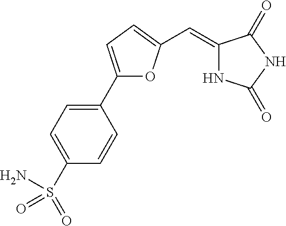 | 0.458 | 0.276 |

TABLE 4

| | AB: PC3 | AB: K-562 | PIM1: | CK2: IC50 |
|---|---|---|---|---|
| Structure | IC50 (μM) | IC50 (μM) | IC50 (μM) | (μM) |
| 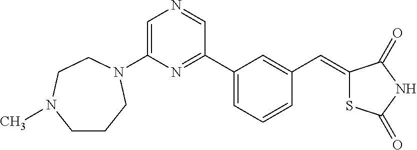 | 9.488 | 0.076 | 0.001 | 0.003 |
| 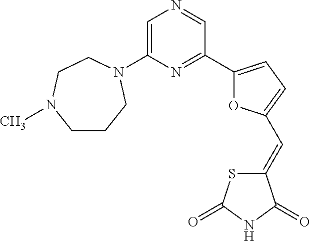 | >10 | 0.146 | 0.022 | 0.006 |
| 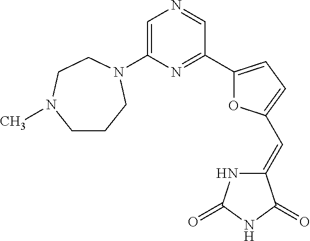 | | | 1.851 | 0.229 |
| 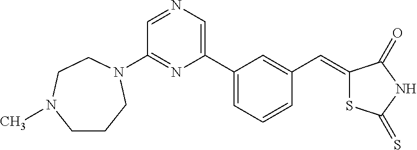 | 3.465 | <3.e−002 | <1.e−003 | 0.001 |
| 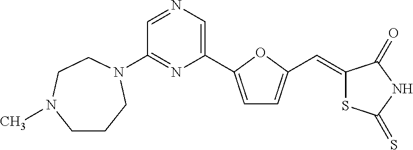 | >10 | | 0.141 | 0.018 |
| 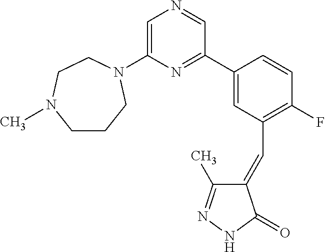 | | | | |
| 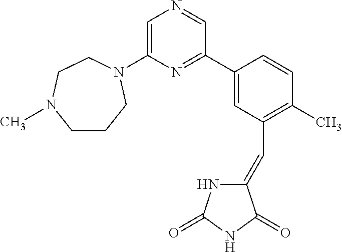 | | | | |
| 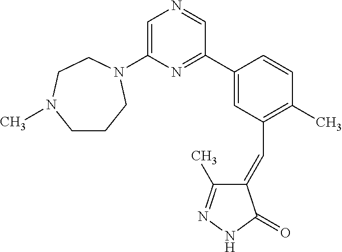 | | | | |
| 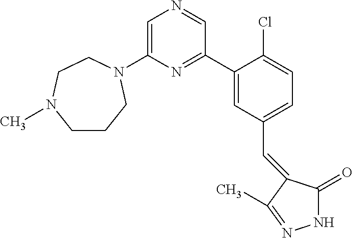 | | | | |

TABLE 5

| | | | AB: | AB: | | | | |
|---|---|---|---|---|---|---|---|---|
| | AB: BxPC3 | AB: MDAMB453 | MV-4-11 | SUM-149PT | AB: | | | |
| Structure | IC50 (uM) | IC50 (uM) | IC50 (uM) | IC50 | K-562 IC50 (uM) | CK2: IC50 (uM) | PIM1: IC50 (uM) | PIM2: IC50 (uM) |
| 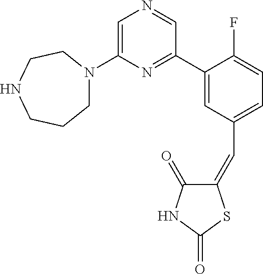 | 4.356 | 1.236 | 1.016 | 2.353 | 2.91 | <0.05 | <0.05 | <0.05 |
| 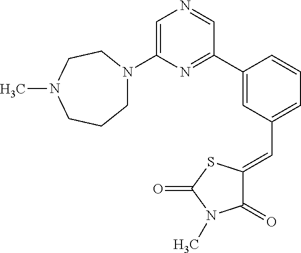 | | | | | | >5 | 0.44 | 2.35 |
| 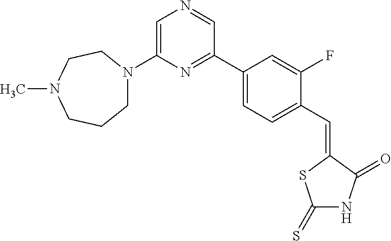 | | | | | | 0.32 | 0.05 | 0.19 |
| 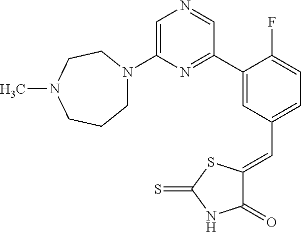 | 2.381 | 0.521 | 0.163 | 0.315 | 0.186 | <0.05 | <0.05 | 0.05 |
| 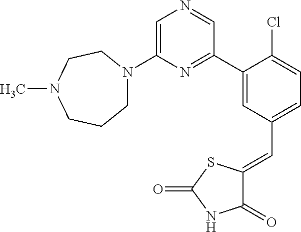 | >30 | 18.388 | 3.01 | >30 | >30 | 0.0921 | <0.05 | <0.05 |
| 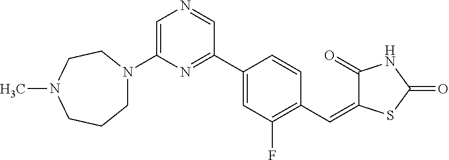 | | | | | | >5 | 1.1833 | >2.5000 |
| 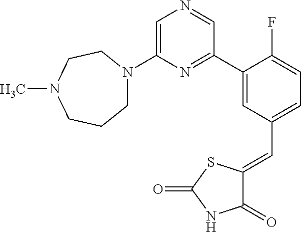 | 6.713 | 1.176 | 0.197 | 1.547 | 0.267 | <0.05 | <0.05 | <0.05 |
| 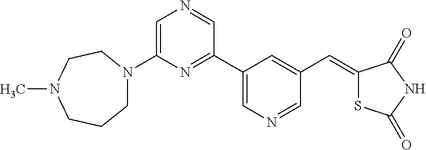 | >30 | 28.3 | 9.351 | >30 | >10 | 0.0106 | <0.05 | <0.05 |
| 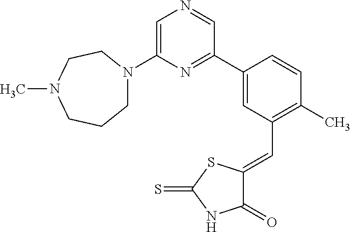 | 16.3 | 22.15 | 2.2 | 25.655 | 9.7 | <0.05 | <0.05 | <0.05 |
| 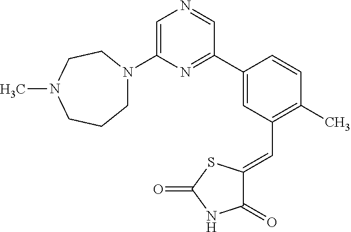 | 25.767 | 15.5 | 3.75 | 8.695 | 6.541 | 0.05 | <0.05 | 0.05 |
| 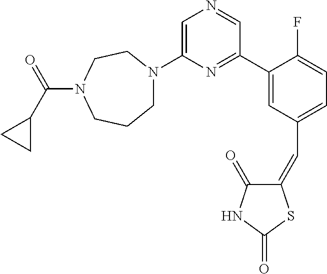 | 23.072 | 4.086 | 2.779 | 3.937 | 0.321 | <0.05 | | <0.05 |
| 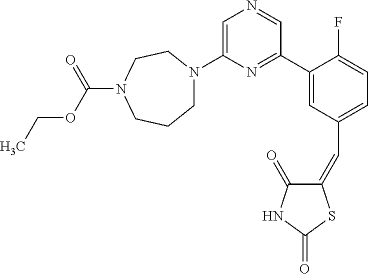 | 22.73 | 10.884 | 3.876 | 4.18 | 3.583 | <0.05 | | <0.05 |
| 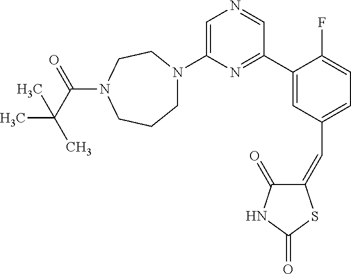 | 12.734 | 0.318 | 0.374 | 1.023 | 0.316 | <0.05 | | <0.05 |
| 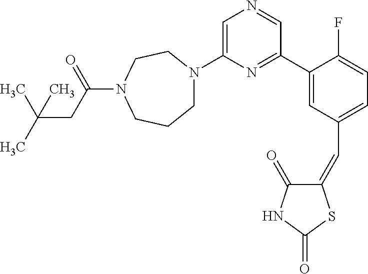 | 10.196 | 2.761 | 3.23 | 1.464 | 1.211 | <0.05 | | <0.05 |
| 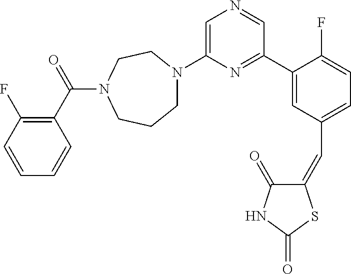 | 19.971 | 2.689 | 3.11 | 2.613 | 1.603 | <0.05 | | <0.05 |
| 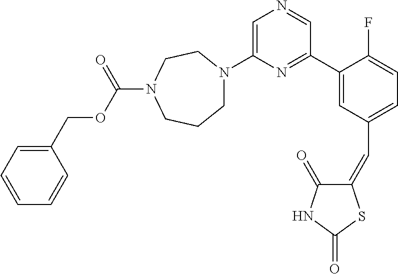 | 24.923 | 3.975 | 3.494 | 8.284 | 2.573 | <0.05 | | <0.05 |
| 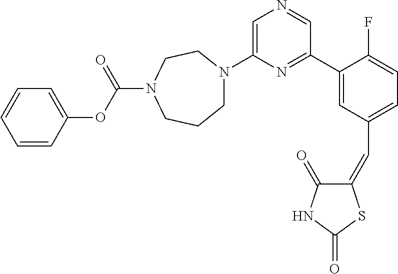 | 23.977 | 3.807 | 6.296 | 6.693 | 9.37 | <0.05 | | <0.05 |
| 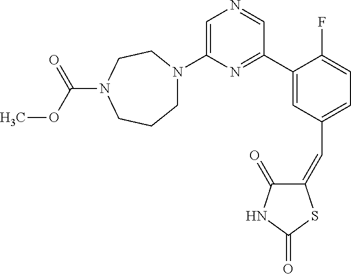 | 18.897 | 3.586 | 2.519 | 2.049 | 3.646 | <0.05 | | <0.05 |
| 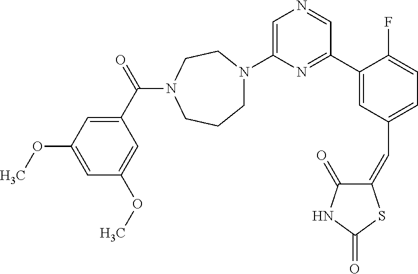 | | | | | | 0.1812 | | 0.1752 |
| 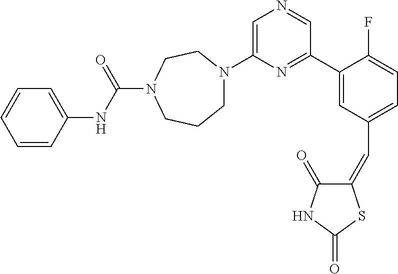 | 18.795 | 10.039 | >10 | 6.154 | 4.3 | <0.05 | | <0.05 |
| 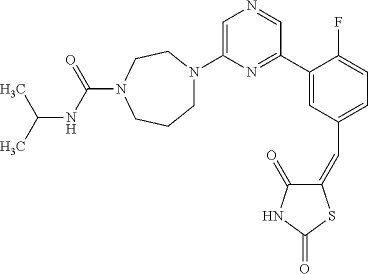 | | 6.283 | 7.7 | 1.564 | | <0.05 | | <0.05 |

TABLE 6

| | | | AB: | | |
|---|---|---|---|---|---|
| | CK2: | PIM2: | BxPC3 | AB: SUM- | AB: |
| | IC50 | % inh | IC50 | 149PT | MDAMB453 |
| Structure | (uM) | @ 2.5 uM | (uM) | IC50 (uM) | IC50 (uM) |
| 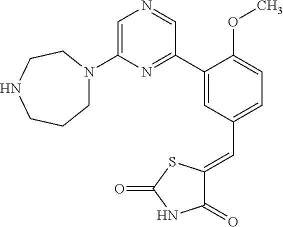 | 0.47175 | 98.491 | | | |
| 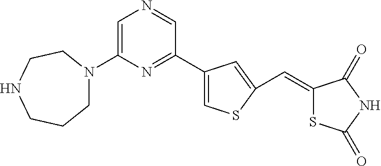 | 0.00439 | 99.881 | >30 | 25.15 | 27.723 |
| 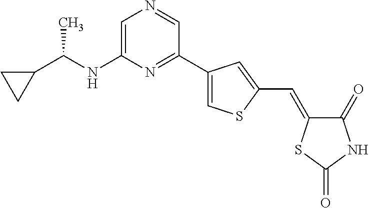 | 0.02396 | 99.438 | 16.736 | 8.723 | 19.161 |
| 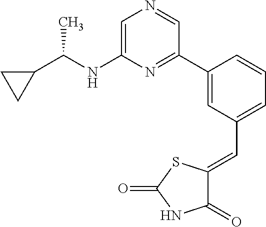 | 0.00144 | 99.886 | 17.204 | 0.703 | 7.208 |

TABLE 7
Additional Compound Examples

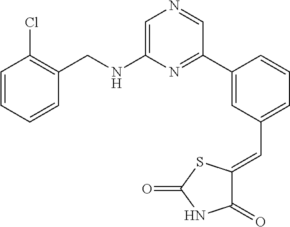

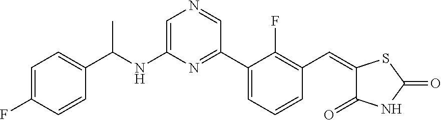

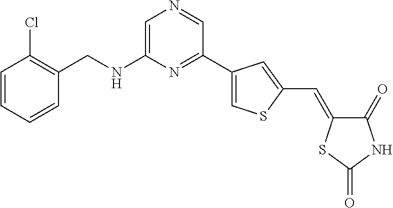

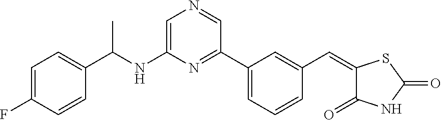

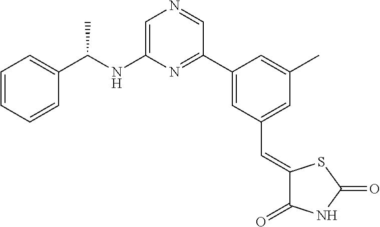

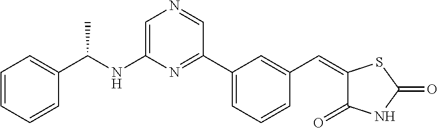

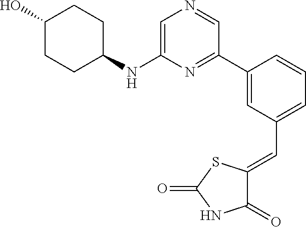

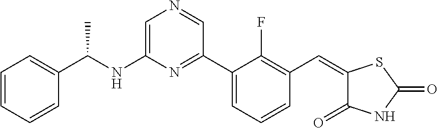

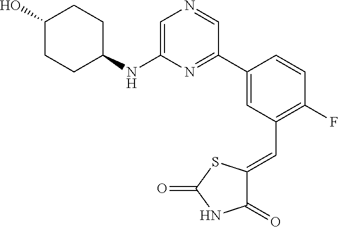

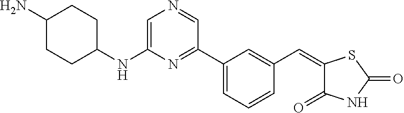

### Example 140

### Representative Embodiments

A1. A compound of Formula (I):

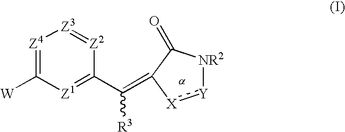

or a pharmaceutically acceptable salt thereof, wherein:

each of Z¹, Z², Z³ and Z⁴ is independently CR¹ or N, provided no more than three of Z¹, Z², Z³ and Z⁴ is N;

each R¹ is independently H, halo, CN, optionally substituted C1-C4 alkyl, optionally substituted C2-C4 alkenyl, optionally substituted C2-C4 alkynyl, optionally substituted C1-C4 alkoxy, or —NR⁶R⁷,
where R⁶ and R⁷ are independently selected from the group consisting of H, optionally substituted C1-C10 alkyl, optionally substituted aryl, optionally substituted arylalkyl, optionally substituted heteroaryl, and optionally substituted heteroarylalkyl, or
R⁶ and R⁷ taken together with the N in —NR⁶R⁷ can form an optionally substituted 5-8 membered ring that optionally contains an additional heteroatom selected from N, O and S as a ring member;

R² is H or C1-C4 alkyl;

R³ is H or optionally substituted C1-C10 alkyl;

α is a single bond,
X is O, S, or NR⁴, where R⁴ is H or an optionally substituted group selected from C1-C4 alkyl, C2-C4 alkenyl, and C2-C4 alkynyl, and
Y is C=Q, where Q is O or S; or

α is a double bond,
X is CR⁵, where R⁵ is H or an optionally substituted group selected from C1-C4 alkyl, C2-C4 alkenyl, and C2-C4 alkynyl, and
Y is N;

W is optionally substituted aryl or optionally substituted heteroaryl, or is —NR¹⁰R¹¹,
wherein said aryl or heteroaryl group may be optionally substituted with a substituent selected from the group consisting of halo, C1-C4 alkyl, C1-C4 alkoxy, CN, —COOR⁸, —CONR⁸R⁹, —CONR⁸NR⁸R⁹, —SO₂NR⁸R⁹, —NR⁸R⁹, —NR⁸COR⁸, and —NR⁸CONR⁸R⁹,
where each R⁸, R⁹, R¹⁰ and R¹¹ is independently selected from the group consisting of H, optionally substituted C1-C10 alkyl, optionally substituted aryl, optionally substituted arylalkyl, optionally substituted heteroaryl, and optionally substituted heteroarylalkyl; or
R⁸ and R⁹ taken together with the N in NR⁸R⁹, and R¹⁰ and R¹¹ taken together with the N in NR¹⁰R¹¹ can independently form an optionally substituted 5-8 membered ring that optionally contains an additional heteroatom selected from N, O and S as a ring member;
provided that when W is phenyl, said phenyl is substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹,
and further provided that when said substituent on phenyl is —SO₂NR⁸R⁹, both of R⁸ and R⁹ are not H.

A2. The compound of embodiment A1, wherein each of Z¹, Z², Z³ and Z⁴ is CR¹.

A3. The compound of embodiment A1, wherein one of Z¹, Z², Z³ and Z⁴ is N and the other three of Z¹, Z², Z³ and Z⁴ are CR¹.

A4. The compound of embodiment A1, wherein two of Z¹, Z², Z³ and Z⁴ are N and the other two of Z¹, Z², Z³ and Z⁴ are CR¹.

A5. The compound of any one of embodiments A1-A4, wherein each R¹ is independently H, Me, halo, OMe, or CF₃.

A6. The compound of any one of embodiments A1-A5, wherein α is a single bond and X is NR⁴ or S.

A7. The compound of any one of embodiments A1-A6, wherein Y is C═O.

A8. The compound of any one of embodiments A1-A6, wherein Y is C═S.

A9. The compound of any one of embodiments A1-A5, wherein α is a double bond, X is CR⁵ and Y is N.

A10. The compound of any one of embodiments A1-A9, wherein R² and R³ are both H.

A11. The compound of any one of embodiments A1-A10, wherein W is optionally substituted aryl or optionally substituted heteroaryl.

A12. The compound of any one of embodiments A1-A11, which is a compound of Formula I-A, I-B, Ia or Ib:

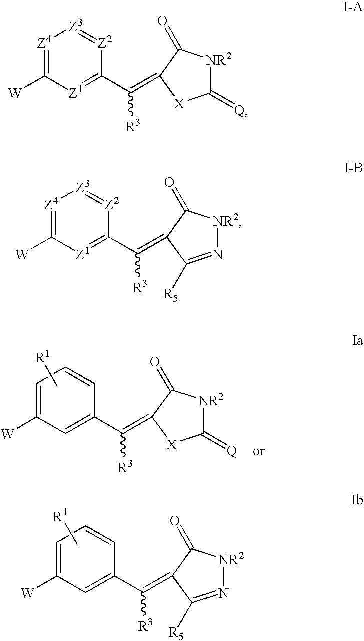

or a pharmaceutically acceptable salt thereof,

wherein Z¹, Z², Z³, Z⁴, R′, R², R³, R⁵, W and X are defined as for Formula I; and

Q is O or S.

A13. The compound of any one of embodiments A1-A12, which is a compound of Formula Ic, Id, Ie or If,

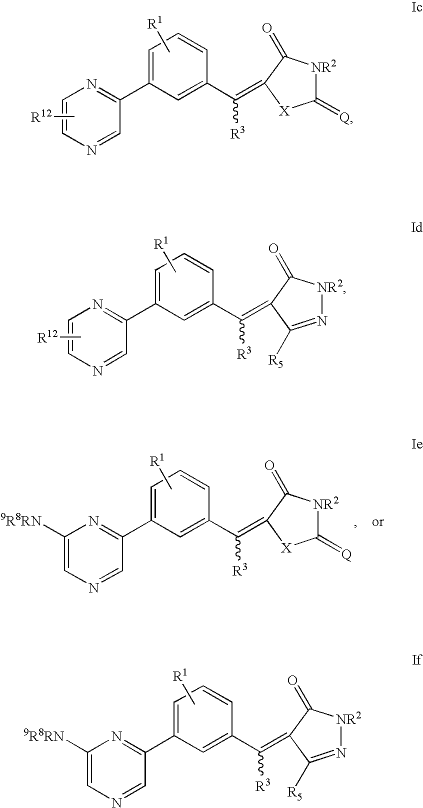

or a pharmaceutically acceptable salt thereof, wherein

R¹, R², R³, R⁵, R⁸, R⁹, and X are defined as for Formula I,

Q is O or S, and

R¹² is —CONR⁸R⁹, —CONR⁸NR⁸R⁹, —SO₂NR⁸R⁹, —NR⁸R⁹, —NR⁸COR⁸, or —NR⁸CONR⁸R⁹.

A14. The compound of any one of embodiments A1-A13, wherein W is optionally substituted phenyl or optionally substituted pyrazine.

A15. The compound of embodiment A14, wherein said phenyl or pyrazine is substituted by —NR⁸R⁹,
where each R⁸ and R⁹ is independently selected from the group consisting of H, optionally substituted C1-C10 alkyl, optionally substituted aryl, optionally substituted arylalkyl, optionally substituted heteroaryl, and optionally substituted heteroarylalkyl; and
where R⁸ and R⁹ taken together with the N in NR⁸R⁹ can form an optionally substituted 5-8 membered ring that optionally contains an additional heteroatom selected from N, O and S as a ring member.

A16. A compound of Formula (II):

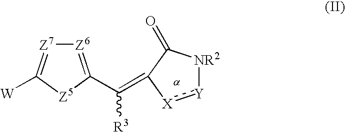

or a pharmaceutically acceptable salt thereof, wherein:

Z⁵ is O, S, or NR²¹, where R²¹ is H or optionally substituted C1-C10 alkyl;

each of Z⁶ and Z⁷ is independently CR¹ or N;

each R¹ is independently H, halo, CN, optionally substituted C1-C4 alkyl, optionally substituted C2-C4 alkenyl, optionally substituted C2-C4 alkynyl, optionally substituted C1-C4 alkoxy, or —NR⁶R⁷,
where R⁶ and R⁷ are independently selected from the group consisting of H, optionally substituted C1-C10 alkyl, optionally substituted aryl, optionally substituted arylalkyl, optionally substituted heteroaryl, and optionally substituted heteroarylalkyl, or
R⁶ and R⁷ taken together with the N in —NR⁶R⁷ can form an optionally substituted 5-8 membered ring that optionally contains an additional heteroatom selected from N, O and S as a ring member;

R² is H or C1-C4 alkyl;

R³ is H or optionally substituted C1-C10 alkyl;

α is a single bond,
X is O, S, or NR⁴, where R⁴ is H or an optionally substituted group selected from C1-C4 alkyl, C2-C4 alkenyl, and C2-C4 alkynyl, and
Y is C=Q, where Q is O or S; or

α is a double bond,
X is CR⁵, where R⁵ is H or an optionally substituted group selected from C1-C4 alkyl, C2-C4 alkenyl, and C2-C4 alkynyl, and
Y is N;

W is optionally substituted aryl or optionally substituted heteroaryl, or is —NR¹⁰R¹¹,
wherein said aryl or heteroaryl group may be optionally substituted with a substituent selected from the group consisting of halo, C1-C4 alkyl, C1-C4 alkoxy, CN, —COOR⁸, —CONR⁸R⁹, —CONR⁸NR⁸R⁹, —SO₂NR⁸R⁹, —NR⁸R⁹, —NR⁸COR⁸, and —NR⁸CONR⁸R⁹,
where each R⁸, R⁹, R¹⁰ and R¹¹ is independently selected from the group consisting of H, optionally substituted C1-C10 alkyl, optionally substituted aryl, optionally substituted arylalkyl, optionally substituted heteroaryl, and optionally substituted heteroarylalkyl; or
R⁸ and R⁹ taken together with the N in NR⁸R⁹, and R¹⁰ and R¹¹ taken together with the N in NR¹⁰R¹¹ can independently form an optionally substituted 5-8 membered ring that optionally contains an additional heteroatom selected from N, O and S as a ring member;
provided that when W is phenyl, said phenyl is substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹,
and further provided that when said substituent on phenyl is —SO₂NR⁸R⁹, both of R⁸ and R⁹ are not H.

A17. The compound of embodiment A16, wherein each of Z⁶ and Z⁷ is CR¹.

A18. The compound of embodiment A16 or A17, wherein Z⁵ is O.

A19. The compound of any one of embodiments A16-A18, wherein each R¹ is independently H, Me, halo, OMe, or CF₃.

A20. The compound of any one of embodiments A16-A19, wherein α is a single bond and X is NR⁴ or S.

A21. The compound of any one of embodiments A16-A20, wherein Y is C═O.

A22. The compound of any one of embodiments A16-A20, wherein Y is C═S.

A23. The compound of any one of embodiments A16-A19, wherein α is a double bond, X is CR⁵ and Y is N.

A24. The compound of any one of embodiments A16-A23, wherein R² and R³ are both H.

A25. The compound of any one of embodiments A16-A24, wherein W is optionally substituted aryl or optionally substituted heteroaryl.

A26. The compound of any one of embodiments A16-A25, which is a compound of Formula II-A, II-B, IIa or IIb:

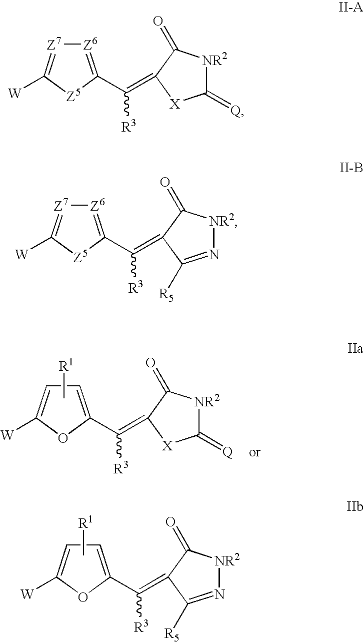

or a pharmaceutically acceptable salt thereof,

wherein Z⁵, Z⁶, Z⁷, R′, R², R³, R⁵, W, and X are as defined in Formula II; and

Q is O or S.

A27. The compound of any one of embodiments A16-A26, which is a compound of Formula IIe, Formula lid, Formula IIe or Formula IIf:

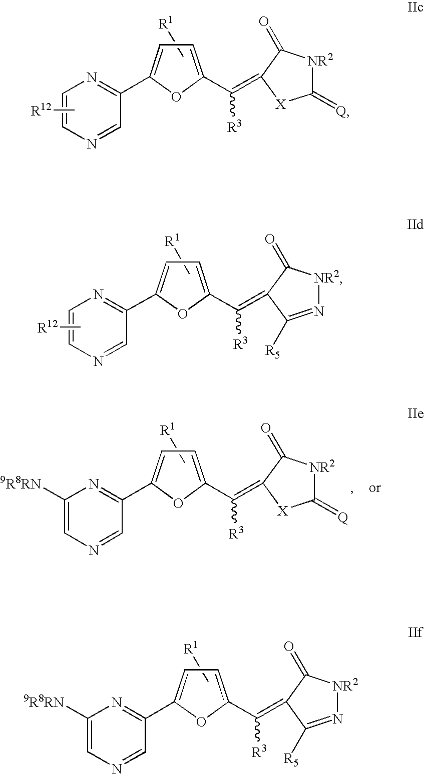

or a pharmaceutically acceptable salt thereof, wherein

R′, R², R³, R⁵, R⁸, R⁹, and X are defined as for Formula II,

Q is O or S, and

R¹² is —CONR⁸R⁹, —CONR⁸NR⁸R⁹, —SO₂NR⁸R⁹, —NR⁸R⁹, —NR⁸COR⁸, or —NR⁸CONR⁸R⁹.

A28. The compound of any one of embodiments A16-A27, wherein W is optionally substituted phenyl or optionally substituted pyrazine.

A29. The compound of embodiment A28, wherein said phenyl or pyrazine is substituted by —NR⁸R⁹,
where each R⁸ and R⁹ is independently selected from the group consisting of H, optionally substituted C1-C10 alkyl, optionally substituted aryl, optionally substituted arylalkyl, optionally substituted heteroaryl, and optionally substituted heteroarylalkyl; or
where R⁸ and R⁹ taken together with the N in NR⁸R⁹ can form an optionally substituted 5-8 membered ring that optionally contains an additional heteroatom selected from N, O and S as a ring member.

A30. A compound of Formula III:

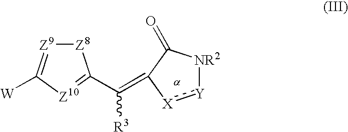

or a pharmaceutically acceptable salt thereof, wherein:

Z⁸ is O, S, or NR³¹, where R³¹ is H or optionally substituted C1-C10 alkyl;

each of Z⁹ and Z¹⁰ is independently CR¹ or N;

each R¹ is independently H, halo, CN, optionally substituted C1-C4 alkyl, optionally substituted C2-C4 alkenyl, optionally substituted C2-C4 alkynyl, optionally substituted C1-C4 alkoxy, or —NR⁶R⁷,
where R⁶ and R⁷ are independently selected from the group consisting of H, optionally substituted C1-C10 alkyl, optionally substituted aryl, optionally substituted arylalkyl, optionally substituted heteroaryl, and optionally substituted heteroarylalkyl, or
R⁶ and R⁷ taken together with the N in —NR⁶R⁷ can form an optionally substituted 5-8 membered ring that optionally contains an additional heteroatom selected from N, O and S as a ring member;

R² is H or C1-C4 alkyl;

R³ is H or optionally substituted C1-C10 alkyl;

α is a single bond,
X is O, S, or NR⁴, where R⁴ is H or an optionally substituted group selected from C1-C4 alkyl, C2-C4 alkenyl, and C2-C4 alkynyl, and
Y is C=Q, where Q is O or S; or

α is a double bond,
X is CR⁵, where R⁵ is H or an optionally substituted group selected from C1-C4 alkyl, C2-C4 alkenyl, and C2-C4 alkynyl, and
Y is N;

W is optionally substituted aryl or optionally substituted heteroaryl, or is —NR¹⁰R¹¹,
wherein said aryl or heteroaryl group may be optionally substituted with a substituent selected from the group consisting of halo, C1-C4 alkyl, C1-C4 alkoxy, CN, —COOR⁸, —CONR⁸R⁹, —CONR⁸NR⁸R⁹, —SO₂NR⁸R⁹, —NR⁸R⁹, —NR⁸COR⁸, and —NR⁸CONR⁸R⁹,
where each R⁸, R⁹, R¹⁰ and R¹¹ is independently selected from the group consisting of H, optionally substituted C1-C10 alkyl, optionally substituted aryl, optionally substituted arylalkyl, optionally substituted heteroaryl, and optionally substituted heteroarylalkyl; or
R⁸ and R⁹ taken together with the N in NR⁸R⁹, and R¹⁰ and R¹¹ taken together with the N in NR¹⁰R¹¹ can independently form an optionally substituted 5-8 membered ring that optionally contains an additional heteroatom selected from N, O and S as a ring member;
provided that when W is phenyl, said phenyl is substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹,
and further provided that when said substituent on phenyl is —SO₂NR⁸R⁹, both of R⁸ and R⁹ are not H.

A31. The compound of embodiment A30, wherein each of Z⁸ is S or O.

A32. The compound of embodiment A30 or A31, wherein each of Z⁹ and Z¹⁰ is CR¹.

A33. The compound of embodiment A30 or A31, wherein at least one of Z⁹ and Z¹⁰ is N.

A34. The compound of any one of embodiments A30-A33, wherein each R¹ is independently H, Me, halo, OMe, or CF₃.

A35. The compound of any one of embodiments A30-A34, wherein α is a single bond and X is NR⁴ or S.

A36. The compound of any one of embodiments A30-A35, wherein Y is C═O.

A37. The compound of any one of embodiments A30-A35, wherein Y is C═S.

A38. The compound of any one of embodiments A30-A34, wherein α is a double bond, X is CR⁵ and Y is N.

A39. The compound of any one of embodiments A30-A38, wherein R² and R³ are both H.

A40. The compound of any one of embodiments A30-A39, wherein W is optionally substituted aryl or optionally substituted heteroaryl.

A41. The compound of any one of embodiments A30-A40, which is a compound of Formula III-A, III-B, IIIa or IIIb:

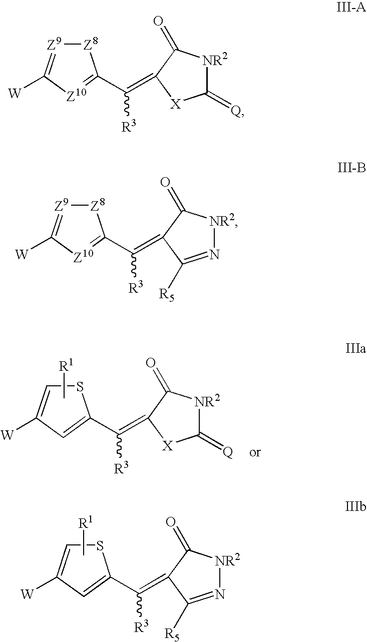

or a pharmaceutically acceptable salt thereof,

wherein Z⁸, Z⁹, Z¹⁰, R¹, R², R³, R⁵, W, and X are defined as for Formula III; and

Q is O or S.

A42. The compound of any one of embodiments A30-A41, which is a compound of Formula IIIc, Formula IIId, Formula IIIe or Formula IIIf:

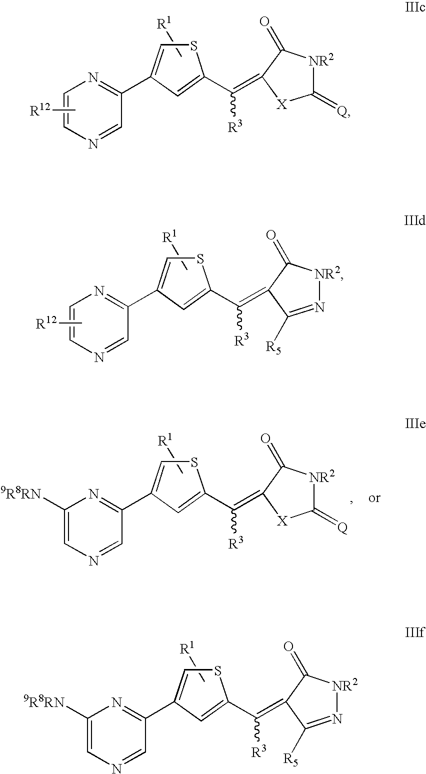

or a pharmaceutically acceptable salt thereof, wherein

R¹, R², R³, R⁵, R⁸, R⁹, and X are defined as for Formula III,

Q is O or S, and

R¹² is —CONR⁸R⁹, —CONR⁸NR⁸R⁹, —SO₂NR⁸R⁹, —NR⁸R⁹, —NR⁸COR⁸, or —NR⁸CONR⁸R⁹.

A43. The compound of any one of embodiments A30-A42, wherein W is optionally substituted phenyl or optionally substituted pyrazine.

A44. The compound of embodiment A43, wherein said phenyl or pyrazine is substituted by —NR⁸R⁹,
where each R⁸ and R⁹ is independently selected from the group consisting of H, optionally substituted C1-C10 alkyl, optionally substituted aryl, optionally substituted arylalkyl, optionally substituted heteroaryl, and optionally substituted heteroarylalkyl; or
where R⁸ and R⁹ taken together with the N in NR⁸R⁹ can form an optionally substituted 5-8 membered ring that optionally contains an additional heteroatom selected from N, O and S as a ring member.

A45. A compound selected from one of the compounds in Table 1, Table 2, Table 3 or Table 4, Table 5, Table 6, or in the Examples, or a pharmaceutically acceptable salt thereof.

A46. A pharmaceutical composition comprising the compound of any one of embodiments A1-A45, admixed with a pharmaceutically acceptable excipient.

A47. A method to treat cancer, a vascular disorder, inflammation, a pathogenic infection, or an immunological disorder, comprising administering to a subject in need of such treatment, an effective amount of the compound of any one of embodiments A1-A45 or a pharmaceutical composition of embodiment A46.

A48. A compound according to any one of embodiments A1-A45 for use as a medicament.

A49. The compound of embodiment A48, wherein the medicament is a medicament for the treatment of cancer, a vascular disorder, inflammation, a pathogenic infection, or an immunological disorder.

A50. Use of a compound of any one of embodiments A1-A45 or a pharmaceutical composition of embodiment A46 in a method to treat cancer, a vascular disorder, inflammation, a pathogenic infection, or an immunological disorder, comprising administering to a subject in need of such treatment.

A51. A compound according to any one of embodiments A1-A50, for use in therapy.

A52. The compound of embodiment A51, for use in the treatment of a vascular disorder, inflammation, a pathogenic infection, or an immunological disorder.

A53. The compound of embodiment A51, for use in the treatment of cancer.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention.

1. A compound of Formula (I): 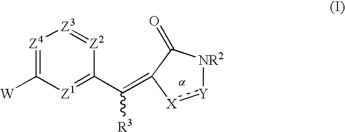
or a pharmaceutically acceptable salt thereof, wherein:
each of Z¹, Z², Z³ and Z⁴ is independently CR¹ or N, provided no more than three of Z¹, Z², Z³ and Z⁴ is N;
each R¹ is independently H, halo, CN, optionally substituted C1-C4 alkyl, optionally substituted C2-C4 alkenyl, optionally substituted C2-C4 alkynyl, optionally substituted C1-C4 alkoxy, or —NR⁶R⁷,
where R⁶ and R⁷ are independently selected from the group consisting of H, optionally substituted C1-C10 alkyl, optionally substituted aryl, optionally substituted arylalkyl, optionally substituted heteroaryl, and optionally substituted heteroarylalkyl, or
R⁶ and R⁷ taken together with the N in —NR⁶R⁷ can form an optionally substituted 5-8 membered ring that optionally contains an additional heteroatom selected from N, O and S as a ring member;
R² is H or C1-C4 alkyl;
R³ is H or optionally substituted C1-C10 alkyl;
α is a single bond,
X is O, S, or NR⁴, where R⁴ is H or an optionally substituted group selected from C1-C4 alkyl, C2-C4 alkenyl, and C2-C4 alkynyl, and
Y is C=Q, where Q is O or S; or
α is a double bond,
X is CR⁵, where R⁵ is H or an optionally substituted group selected from C1-C4 alkyl, C2-C4 alkenyl, and C2-C4 alkynyl, and
Y is N;
W is optionally substituted aryl or optionally substituted heteroaryl, or is —NR¹⁰R¹¹,
wherein said aryl or heteroaryl group may be optionally substituted with a substituent selected from the group consisting of halo, C1-C4 alkyl, C1-C4 alkoxy, CN, —COOR⁸, —CONR⁸R⁹, —CONR⁸NR⁸R⁹, —SO₂NR⁸R⁹, —NR⁸R⁹, —NR⁸COR⁸, and —NR⁸CONR⁸R⁹,
where each R⁸, R⁹, R¹⁰ and R¹¹ is independently selected from the group consisting of H, optionally substituted C1-C10 alkyl, optionally substituted aryl, optionally substituted arylalkyl, optionally substituted heteroaryl, and optionally substituted heteroarylalkyl; or
R⁸ and R⁹ taken together with the N in NR⁸R⁹, and R¹⁰ and R¹¹ taken together with the N in NR¹⁰R¹¹ can independently form an optionally substituted 5-8 membered ring that optionally contains an additional heteroatom selected from N, O and S as a ring member;
provided that when W is phenyl, said phenyl is substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹,
and further provided that when said substituent on phenyl is —SO₂NR⁸R⁹, both of R⁸ and R⁹ are not H.

2. The compound of claim 1, wherein each of Z¹, Z², Z³ and Z⁴ is CR¹.

3. The compound of claim 1, wherein at least one of Z¹, Z², Z³ and Z⁴ is N.

4. The compound of claim 1, wherein α is a single bond and X is S.

5. The compound of claim 1, wherein W is optionally substituted aryl or optionally substituted heteroaryl.

6. The compound of claim 1, which is a compound of Formula I-A: 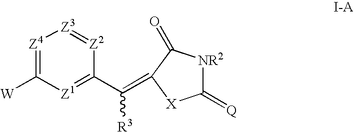
or a pharmaceutically acceptable salt thereof,
wherein W is optionally substituted aryl or optionally substituted heteroaryl; and
Q is O or S.

7. A compound of Formula (II): 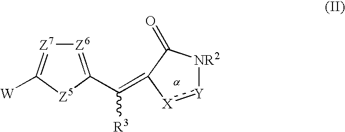
or a pharmaceutically acceptable salt thereof, wherein:
Z⁵ is O, S, or NR²¹, where R²¹ is H or optionally substituted C1-C10 alkyl;
each of Z⁶ and Z⁷ is independently CR¹ or N;
each R¹ is independently H, halo, CN, optionally substituted C1-C4 alkyl, optionally substituted C2-C4 alkenyl, optionally substituted C2-C4 alkynyl, optionally substituted C1-C4 alkoxy, or —NR⁶R⁷,
where R⁶ and R⁷ are independently selected from the group consisting of H, optionally substituted C1-C10 alkyl, optionally substituted aryl, optionally substituted arylalkyl, optionally substituted heteroaryl, and optionally substituted heteroarylalkyl, or
R⁶ and R⁷ taken together with the N in —NR⁶R⁷ can form an optionally substituted 5-8 membered ring that optionally contains an additional heteroatom selected from N, O and S as a ring member;
R² is H or C1-C4 alkyl;
R³ is H or optionally substituted C1-C10 alkyl;
α is a single bond,
X is O, S, or NR⁴, where R⁴ is H or an optionally substituted group selected from C1-C4 alkyl, C2-C4 alkenyl, and C2-C4 alkynyl, and
Y is C=Q, where Q is O or S; or
α is a double bond,
X is CR⁵, where R⁵ is H or an optionally substituted group selected from C1-C4 alkyl, C2-C4 alkenyl, and C2-C4 alkynyl, and
Y is N;
W is optionally substituted aryl or optionally substituted heteroaryl, or is —NR¹⁰R¹¹,
wherein said aryl or heteroaryl group may be optionally substituted with a substituent selected from the group consisting of halo, C1-C4 alkyl, C1-C4 alkoxy, CN, —COOR⁸, —CONR⁸R⁹, —CONR⁸NR⁸R⁹, —SO₂NR⁸R⁹, —NR⁸R⁹, —NR⁸COR⁸, and —NR⁸CONR⁸R⁹,
where each R⁸, R⁹, R¹⁰ and R¹¹ is independently selected from the group consisting of H, optionally substituted C1-C10 alkyl, optionally substituted aryl, optionally substituted arylalkyl, optionally substituted heteroaryl, and optionally substituted heteroarylalkyl; or
R⁸ and R⁹ taken together with the N in NR⁸R⁹, and R¹⁰ and R¹¹ taken together with the N in NR¹⁰R¹¹ can independently form an optionally substituted 5-8 membered ring that optionally contains an additional heteroatom selected from N, O and S as a ring member;
provided that when W is phenyl, said phenyl is substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹,
and further provided that when said substituent on phenyl is —SO₂NR⁸R⁹, both of R⁸ and R⁹ are not H.

8. The compound of claim 7, wherein each of Z⁶ and Z⁷ is CR¹ and Z⁵ is O.

9. The compound of claim 7, wherein α is a single bond and X is S.

10. The compound of claim 7, wherein W is optionally substituted aryl or optionally substituted heteroaryl.

11. The compound of claim 7, which is a compound of Formula II-A: 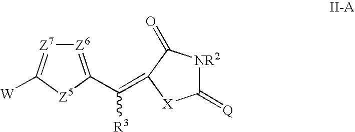
or a pharmaceutically acceptable salt thereof,
wherein W is optionally substituted aryl or optionally substituted heteroaryl; and
Q is O or S.

12. A compound of Formula (III): 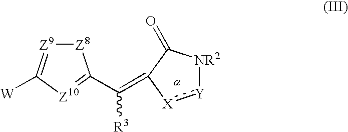
or a pharmaceutically acceptable salt thereof, wherein:
Z⁸ is O, S, or NR³¹, where R³¹ is H or optionally substituted C1-C10 alkyl;
each of Z⁹ and Z¹⁰ is independently CR¹ or N;
each R¹ is independently H, halo, CN, optionally substituted C1-C4 alkyl, optionally substituted C2-C4 alkenyl, optionally substituted C2-C4 alkynyl, optionally substituted C1-C4 alkoxy, or —NR⁶R⁷,
where R⁶ and R⁷ are independently selected from the group consisting of H, optionally substituted C1-C10 alkyl, optionally substituted aryl, optionally substituted arylalkyl, optionally substituted heteroaryl, and optionally substituted heteroarylalkyl, or
R⁶ and R⁷ taken together with the N in —NR⁶R⁷ can form an optionally substituted 5-8 membered ring that optionally contains an additional heteroatom selected from N, O and S as a ring member;
R² is H or C1-C4 alkyl;
R³ is H or optionally substituted C1-C10 alkyl;
α is a single bond,
X is O, S, or NR⁴, where R⁴ is H or an optionally substituted group selected from C1-C4 alkyl, C2-C4 alkenyl, and C2-C4 alkynyl, and
Y is C=Q, where Q is O or S; or
α is a double bond,
X is CR⁵, where R⁵ is H or an optionally substituted group selected from C1-C4 alkyl, C2-C4 alkenyl, and C2-C4 alkynyl, and
Y is N;
W is optionally substituted aryl or optionally substituted heteroaryl, or is —NR¹⁰R¹¹,
wherein said aryl or heteroaryl group may be optionally substituted with a substituent selected from the group consisting of halo, C1-C4 alkyl, C1-C4 alkoxy, CN, —COOR⁸, —CONR⁸R⁹, —CONR⁸NR⁸R⁹, —SO₂NR⁸R⁹, —NR⁸R⁹, —NR⁸COR⁸, and —NR⁸CONR⁸R⁹,
where each R⁸, R⁹, R¹⁰ and R¹¹ is independently selected from the group consisting of H, optionally substituted C1-C10 alkyl, optionally substituted aryl, optionally substituted arylalkyl, optionally substituted heteroaryl, and optionally substituted heteroarylalkyl; or
R⁸ and R⁹ taken together with the N in NR⁸R⁹, and R¹⁰ and R¹¹ taken together with the N in NR¹⁰R¹¹ can independently form an optionally substituted 5-8 membered ring that optionally contains an additional heteroatom selected from N, O and S as a ring member;
provided that when W is phenyl, said phenyl is substituted with at least one substituent selected from the group consisting of —CONR⁸R⁹, —CONR⁸NR⁸R⁹, —SO₂NR⁸R⁹, —NR⁸R⁹, —NR⁸COR⁸, and —NR⁸CONR⁸R⁹,
and further provided that when said substituent on phenyl is —SO₂NR⁸R⁹, both of R⁸ and R⁹ are not H.

13. The compound of claim 12, wherein Z⁸ is S and each of Z⁹ and Z¹⁰ is CR¹.

14. The compound of claim 12, wherein α is a single bond and X is S.

15. The compound of claim 12, wherein W is optionally substituted aryl or optionally substituted heteroaryl.

16. The compound of claim 12, which is a compound of Formula III-A: 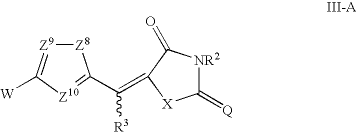
or a pharmaceutically acceptable salt thereof,
wherein W is optionally substituted aryl or optionally substituted heteroaryl; and
Q is O or S.

17. A compound selected from the compounds disclosed in Tables 1-7 or in the Examples, or a pharmaceutically acceptable salt thereof.

18. A pharmaceutical composition comprising the compound of claim 1, admixed with a pharmaceutically acceptable excipient.

19. A pharmaceutical composition comprising the compound of claim 7, admixed with a pharmaceutically acceptable excipient.

20. A pharmaceutical composition comprising the compound of claim 12, admixed with a pharmaceutically acceptable excipient.

21. A method to treat cancer, a vascular disorder, inflammation, a pathogenic infection, or an immunological disorder, comprising administering to a subject in need of such treatment an effective amount of the compound of claim 1.

22. A method to treat cancer, a vascular disorder, inflammation, a pathogenic infection, or an immunological disorder, comprising administering to a subject in need of such treatment, an effective amount of the compound of claim 7.

23. A method to treat cancer, a vascular disorder, inflammation, a pathogenic infection, or an immunological disorder, comprising administering to a subject in need of such treatment, an effective amount of the compound of claim 12.


SEQUENCE LISTING (6 sequences)

SEQ ID NO: 1 - protein, 391 aa, Homo sapiens
MSGPVPSRARVYTDVNTHRPREYWDYESHVVEWGNQDDYQLVRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMPVPAAAGAQQ

SEQ ID NO: 2 - protein, 391 aa, Homo sapiens; identical to SEQ ID NO: 1

SEQ ID NO: 3 - protein, 255 aa, Homo sapiens
MYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMPVPAAAGAQQ

SEQ ID NO: 4 - protein, 10 aa, Unknown
  Substrate peptide
RRRDDDSDDD

SEQ ID NO: 5 - protein, 9 aa, Unknown
  Substrate peptide
KKRNRTLTK

SEQ ID NO: 6 - protein, 10 aa, Unknown
  Substrate peptide
RSRSSYPAGT